(19) United States
(12) Patent Application Publication
(10) Pub. No.: US 2024/0043389 A1
(43) Pub. Date: Feb. 8, 2024

(54) COMPOUNDS AND METHODS FOR INHIBITION OF BAX-MEDIATED CELL DEATH

(71) Applicant: Albert Einstein College of Medicine, Bronx, NY (US)

(72) Inventor: Evripidis Gavathiotis, Roslyn, NY (US)

(73) Assignee: Albert Einstein College of Medicine, Bronx, NY (US)

(21) Appl. No.: 18/043,441

(22) PCT Filed: Sep. 1, 2021
(86) PCT No.: PCT/US21/48695

§ 371 Date: Feb. 28, 2023

Related U.S. Application Data
(60) Provisional application No. 63/073,256, filed on Sep. 1, 2020.

(51) Int. Cl. C07D 231/46; C07D 401/04; C07D 417/04; A61K 31/635; A61P 7/04; A61K 31/4152; A61K 31/4439; A61K 31/427; A61K 31/4155; A01N 1/02

(52) U.S. Cl. CPC C07D 231/46; C07D 401/04; C07D 417/04; A61K 31/635; A61P 7/04; A61K 31/4152; A61K 31/4439; A61K 31/427; A61K 31/4155; A01N 1/0226

(57) ABSTRACT

This disclosure provides novel compounds and methods for inhibiting BAX activity and BAX-mediated apoptosis, as well as compounds and methods for treating or preventing BAX-mediated disorders.

### CROSS-REFERENCE TO RELATED APPLICATIONS

This application claims priority under 35 U.S.C. § 119(e) to U.S. Provisional Patent Application No. 63/073,256, filed Sep. 1, 2020. The foregoing application is incorporated by reference herein in its entirety.

### FIELD OF THE INVENTION

This disclosure relates generally to compounds and methods for inhibiting BAX activity and BAX-mediated apoptosis and for treating or preventing BAX-mediated disorders.

### BACKGROUND OF THE INVENTION

BCL-2 family proteins are principal regulators of apoptosis in health and diseases. Mitochondrial outer membrane permeabilization (MOMP) is a key event that defines apoptotic cell death. MOMP releases apoptogenic factors, such as cytochrome c, into the cytosol, which in turn irreversibly execute the apoptotic signaling cascade. Pro-apoptotic BCL-2 proteins BAX and BAK play a key role in this process due to their ability to transform into mitochondrial outer membrane-embedded oligomers that induce MOMP. In cells, BAX and BAK can exist as an inactive monomer, autoinhibited homodimer, or a neutralized conformation bound to anti-apoptotic BCL-2 family members such as BCL-2, BCL-xL, and MCL-1 (Edlich, F. et al. Cell 145, 104-116 (2011); Garner, T. P. et al. Mol. Cell 63, 485-497 (2016)). The pro-apoptotic “BCL-2 homology 3 (B1H3)-only” proteins such as BIM, BID, and PUMA, which comprise the third class of the BCL-2 family, sense cellular stress and utilize their BH3 domain helix to either neutralize the anti-apoptotic BCL-2 proteins and/or directly activate pro-apoptotic BAX and BAK and initiate their conformational transformation (Chen, H. C. et al. Nat Cell Biol 17, 1270-1281, (2015)).

BAX activation is a dynamic process that occurs upon binding of a BH3-only protein with its BH3 domain helix to the N-terminal BAX trigger site (α1, α6 helices), inducing several conformational changes (Suzuki, M., et al. Cell 103, 645-654 (2000)). The release of the helix α9 from the C-terminal canonical site of BAX, formed by α3-α5 helices, allows BAX to translocate from the cytosol and insert into the mitochondrial outer membrane (MOM) (Gavathiotis, E., et al. Mol. Cell 40, 481-492 (2010)). Once translocated, BAX undergoes homo-oligomerization, which then permeabilizes the MOM (Czabotar, P. E., et al. Cell 152, 519-531 (2013)).

While BAX-mediated cell death contributes to tissue homeostasis and killing of malfunctioned cells, genetic deletion of BAX alone has shown successful protection from excess cell death in various disease models (Fuchs, Y. & Steller, H. Cell 147, 742-758 (2011), Singh, R., et al. Nat Rev Mol Cell Biol 20, 175-193, (2019). Thus, small molecules that can directly modulate BAX can be useful probes to investigate the role of BAX in the context of various biological mechanisms and disease models (Garner, T. P., et al. Curr. Opin. Chem. Biol. 39, 133-142 (2017)). Such chemical probes can also aid in identifying BAX regulatory sites and elucidate the complex conformational transformation of BAX.

Accordingly, there exists a strong need for agents and methods for modulating (e.g., inhibiting) BAX activity and BAX-mediated apoptosis.

### SUMMARY OF THE INVENTION

This disclosure addresses the need mentioned above in a number of aspects. In one aspect, this disclosure provides a compound of Formula (I):

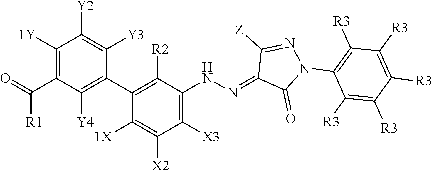

or
Formula (II):

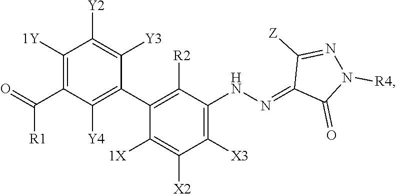

or a stereoisomer or a pharmaceutically acceptable salt thereof,
wherein:
R1 is selected from OH, O—CH₃, O—CH₂CH₃, O—CH(CH₃)₂, NH—CH₃, and NH—CH₂CH₃;
R2 is selected from H, F, Cl, CH₃, CF₃, OCH₃, CH₂CH₃, OCH₂CH₃;
R3 is selected from H, OH, CH₃, CF₃, NH₂, F, Cl, OCH₃, and NHCOCH₃;
X1, X2, or X3 is selected from H, F, Cl, OH, CH₃, CF₃, CH₂CH₃, OCH₃, OCH₂CH₃, and —CO—CH₃;
Y1, Y2, Y3, or Y4 is selected from H, F, Cl, CH₃, CF₃, OCH₃, and CH₂CH₃;
Z is selected from H, F, Cl, CH₃, CF₃, OCH₃, CH₂CH₃, CH₂NH₂, and CH₂CH₂NH₂;
R4 is selected from:

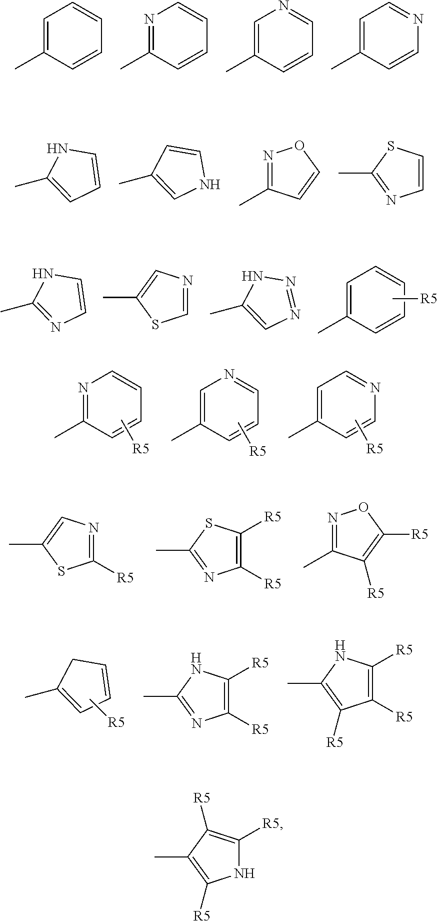

R5 is selected from H, OH, CH₃, CF₃, NH₂, F, Cl, OCH₃, and NHCOCH₃.

In some embodiments, the compound is selected from:

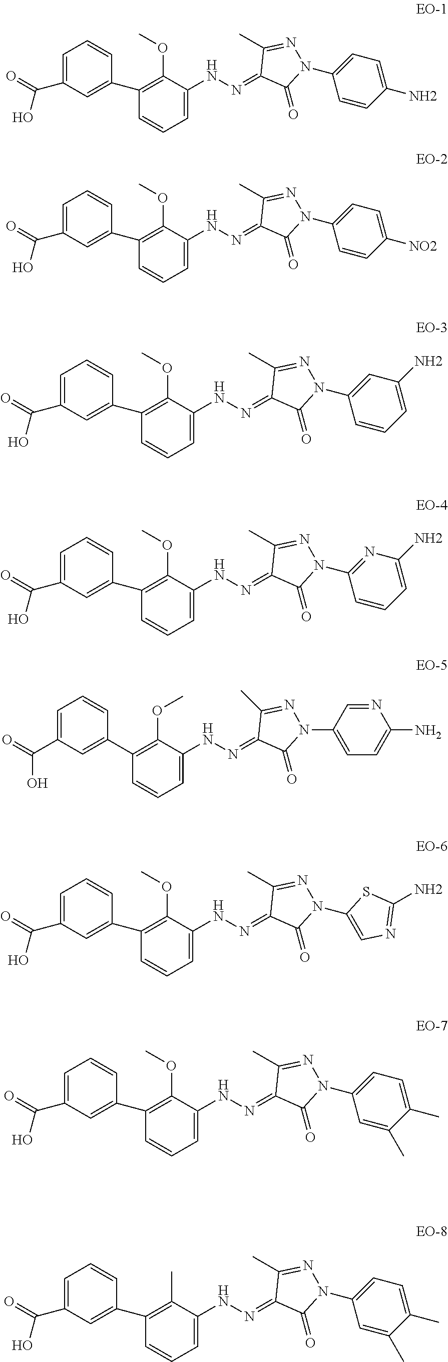

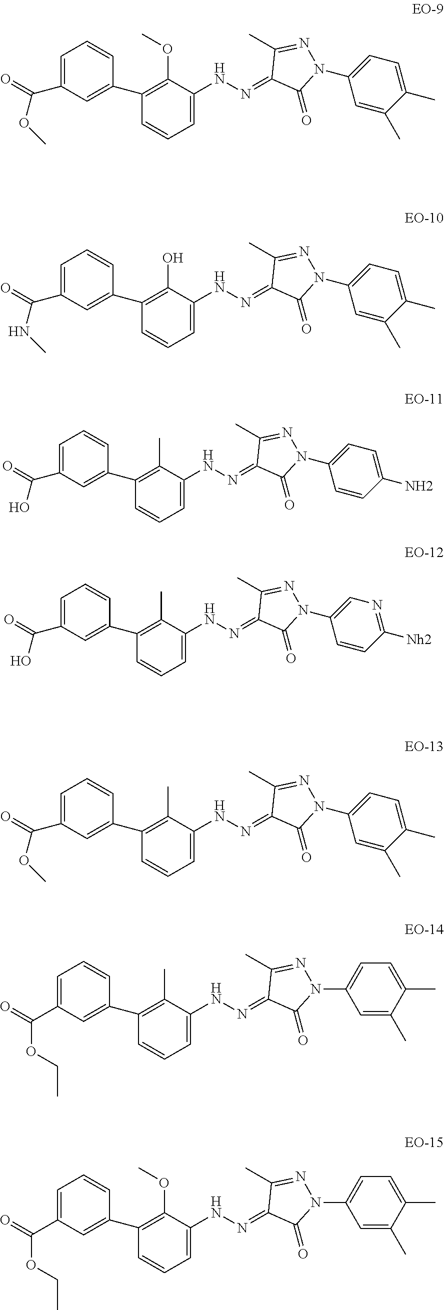

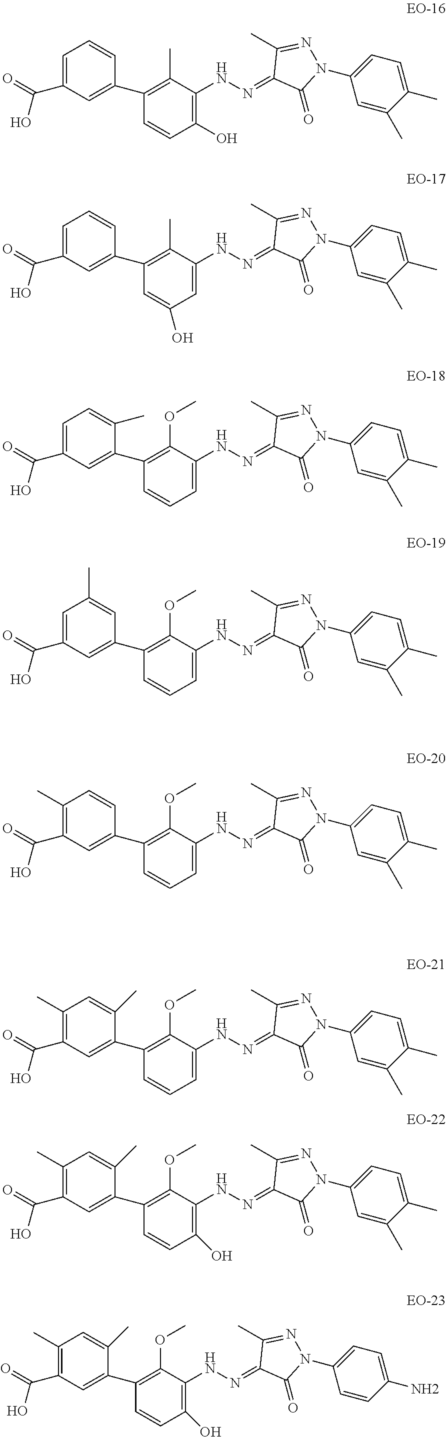

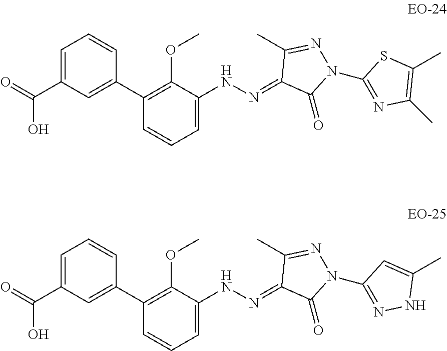 [SMILES: COc1c(N/N=C2/C(=O)N(c3nc(C)c(C)s3)N=C2C)cccc1-c1cccc(C(=O)O)c1 ; COc1c(N/N=C2/C(=O)N(c3cc(C)[nH]n3)N=C2C)cccc1-c1cccc(C(=O)O)c1]

Also provided in this disclosure is a pharmaceutical composition comprising (i) a compound described above, or a stereoisomer or a pharmaceutically acceptable salt thereof, and (ii) a pharmaceutically acceptable carrier.

In another aspect, this disclosure also provides a method of treating or preventing a disorder mediated by BAX in a subject. The method comprises administering to the subject a therapeutically effective amount of a compound described above or a stereoisomer or a pharmaceutically acceptable salt thereof, or the pharmaceutical composition, as described above. In some embodiments, the subject is a mammal, e.g., a human.

In some embodiments, the disorder is associated with increased expression or activation of the BAX protein. In some embodiments, the disorder comprises a neuronal disorder or an autoimmune disease. In some embodiments, the neuronal disorder is selected from epilepsy, multiple sclerosis, Alzheimer's disease, Huntington's disease, Parkinson's disease, retinal diseases, spinal cord injury, Crohn's disease, head trauma, spinocerebellar ataxias, and dentatorubral-pallidoluysian atrophy. In some embodiments, the autoimmune disease is selected from Multiple Sclerosis, amyotrophic lateral sclerosis, retinitis pigmentosa, inflammatory bowel disease (IBD), rheumatoid arthritis, asthma, septic shock, transplant rejection, and AIDS.

In some embodiments, the disorder comprises ischemia (e.g., stroke, myocardial infarction, and reperfusion injury), cardiomyopathy, chemotherapy-induced cardiotoxicity, chemotherapy-induced cardiomyopathy, cardiovascular disorders, arteriosclerosis, heart failure, viral infection heart injury, myocarditis, viral infection lung injury, heart transplantation, renal hypoxia, a liver disease, a kidney disease, an intestinal disease, liver ischemia, intestinal ischemia, acute optic nerve damage, glaucoma, Idiopathic pulmonary fibrosis (IPF), chemotherapy-induced ocular toxicity, hepatitis, viral infection.

In some embodiments, the method further comprises administering to the subject a second therapeutic agent or therapy. In some embodiments, the second therapeutic agent comprises an anti-inflammatory agent or an anti-tumor/anti-cancer agent. In some embodiments, the anti-tumor/anti-cancer agent is navitoclax. In some embodiments, the second therapeutic agent is administered to the subject before, after, or concurrently with the compound or a stereoisomer or a pharmaceutically acceptable salt thereof, or the pharmaceutical composition, as described above.

In some embodiments, the subject was previously administered an anti-cancer therapy. In some embodiments, the anti-cancer therapy comprises surgery, radiation, chemotherapy, and/or immunotherapy. In some embodiments, the chemotherapy comprises a therapeutic agent that inhibits Bcl-xL. In some embodiments, the therapeutic agent that inhibits Bcl-xL is navitoclax.

In some embodiments, the compound or a stereoisomer or a pharmaceutically acceptable salt thereof, or the pharmaceutical composition, as described above, is administered intratumorally, intravenously, subcutaneously, intraosseously, orally, transdermally, in sustained release, in controlled release, in delayed release, as a suppository, or sublingually. In some embodiments, the compound or a stereoisomer or a pharmaceutically acceptable salt thereof, or the pharmaceutical composition, as described above, is administered prophylactically or therapeutically.

In another aspect, this disclosure further provides a method of treating or ameliorating a symptom of thrombocytopenia associated with treatment targeting Bcl-xL. The method comprises:
(i) selecting a subject having a condition treatable by a therapeutic agent that inhibits Bcl-xL; and
(ii) administering to the subject a therapeutically effective amount of a compound or a stereoisomer or a pharmaceutically acceptable salt thereof, or a pharmaceutical composition, as described above, in combination with a therapeutically effective amount of the therapeutic agent.

In some embodiments, the therapeutic agent that inhibits Bcl-xL is navitoclax. In some embodiments, the condition is a cancer or an autoimmune disease. In some embodiments, the therapeutic agent is administered to the subject before, after, or concurrently with the compound or a stereoisomer or a pharmaceutically acceptable salt thereof, or the pharmaceutical composition, as described above.

In some embodiments, a therapeutic agent or a compound or a stereoisomer or a pharmaceutically acceptable salt thereof, or a pharmaceutical composition, as described above, is administered to the subject in one or more doses.

In another aspect, this disclosure additionally provides a method of inhibiting BAX-mediated apoptosis in a cell. The method comprises administering to the cell expressing a BAX protein an effective amount of a compound or a stereoisomer or a pharmaceutically acceptable salt thereof, or a pharmaceutical composition, as described above.

In yet another aspect, this disclosure further provides a method of inhibiting activation or function of the BAX protein in a subject, a cell, or a biological sample thereof. The method comprises (i) administering to the subject or the cell a therapeutically effective amount of a compound or a stereoisomer or a pharmaceutically acceptable salt thereof, or a pharmaceutical composition, as described above; or (ii) contacting the biological sample with a compound or a stereoisomer or a pharmaceutically acceptable salt thereof, or a pharmaceutical composition, as described above.

In some embodiments, the cell is a neuronal cell or a cardiac cell. In some embodiments, the activation of BAX protein is mediated by Bim, Bid, Bmf, Puma, or Noxa.

In another aspect, this disclosure provides a method for preserving or treating an organ or tissue. The method comprises contacting the organ or tissue with an effective amount of a compound or a stereoisomer or a pharmaceutically acceptable salt thereof as described herein, under conditions effective for preservation of the organ or tissue.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1a, 1b, 1c, 1d, 1e, 1f, and 1g are a set of diagrams showing that eltrombopag (EO) potently binds BAX. FIG. 1a shows a surface representation of the inactive BAX structure (PDB 1F16) depicting the location of the BAX trigger site and closed α1-α2 loop. FIGS. 1b and 1c show surface representations of the BIM BH3-bound active conformation (PDB 2K7W) depicting binding of BIM BH3 with opened α1-α2 loop at the trigger site (FIG. 1b) and the location of α9 bound to the C-terminal canonical groove (FIG. 1c). FIG. 1d shows chemical structures of BAM7, BTSA1, and EO derived by similarity search. FIG. 1e shows the results of the competitive fluorescence polarization (FP) binding assay. Data are representative of three independent experiments, each n=3±SEM. FIG. 1f shows binding affinity of FITC-BIM-SAHB to BAX by FP in the presence of EO. Data represent n=2±SEM from two independent experiments. FIG. 1g shows microscale thermophoresis (MST) direct binding of EO to BAX-4C. Data are representative of three independent experiments, each n=3±SEM.

FIGS. 2a, 2b, 2c, 2d, 2e, 2f, 2g, 2h, and 2i are a set of graphs showing that EO inhibits BAX activation. FIGS. 2a, 2b, 2c, 2d, and 2e show the results of the BAX-mediated membrane permeabilization assay using liposomes with 50 nM BAX and 5 nM tBID (FIGS. 2a, 2b, and 2c), 50 nM BAX and 1 μM BIM-B1H3 (FIG. 2d), or 250 nM BAX at 42° C. (FIG. 2e), each at 30 minutes. Data are representative of three independent experiments, each n=3±SEM. FIG. 2f shows summary percentage inhibition curve for all liposomal release stimuli with IC50 included for clarity. FIGS. 2g and 2h show the results for the membrane translocation assay using NBD-labeled BAX (800 nM) activated by tBID (200 nM) (FIG. 2g) and BIM BH3 (1 μM) (FIG. 2h), each at 120 minutes. Data are representative of three independent experiments, each n=3±SEM. FIG. 2i shows summary percentage inhibition curves for all BAX translocation stimuli with IC50 included for clarity. Two-sided t-test, ****P<0.0001; ***P<0.001; **P<0.01; *P<0.05; ns, P>0.05.

FIGS. 3a, 3b, 3c, 3d, 3e, 3f, 3g, and 3h are a set of diagrams showing that EO binds the BAX trigger using unique contacts. FIG. 3a shows measured chemical shift perturbations (CSPs) of ¹⁵N-labeled BAX in the presence of 1:2 BAX:EO, plotted as a function of BAX residue number. Residues with chemical shift perturbations over the significance threshold or two times the significance threshold are labeled light blue or dark blue, respectively. The black dotted line represents the average CSP. Basic residues of the N-terminal trigger site are labeled for clarity. Data are representative of three independent experiments. FIG. 3b shows mapping of residues undergoing significant CSPs to the surface and the ribbon structure of BAX (PDB: 1F16). Residues with significant CSPs cluster on the N-terminal trigger site of BAX surrounding a hydrophobic pocket formed by α1 and α6. FIGS. 3c and 3d show percent inhibition of BAX-mediated membrane permeabilization assay using liposomes with 250 nM BAX and 5 nM tBID with various trigger site mutants, dose-response IC50 (FIG. 3c) and bar graph for 5 μM EO (FIG. 3d) are shown for clarity. Data are representative of two independent experiments, each n=3±SEM. FIG. 3e shows transparent surface with ribbon representation of the EO binding site as determined by NMR data and docking. FIG. 3f shows close-up view of the EO binding site with residues determined by NMR data forming hydrophobic contacts with EO are highlighted, and residues forming specific interactions, R134 and R145, are highlighted. FIG. 3g shows BAX electrostatic surface representation highlighting positive and negative charges as a gradient. FIG. 3h shows the results of the competitive fluorescence polarization binding assay of EO and inactive EO-methyl ester analog. Data are representative of three independent experiments n=3±SEM. Two-sided t-test, ****P<0.0001; ***P<0.001; **P<0.01; *P<0.05; ns, P>0.05.

FIGS. 4a, 4b, 4c, 4d, 4e, 4f, 4g, and 4h are a set of diagrams showing that EO stabilizes an inactive BAX structure. FIG. 4a shows an overlay of structures of BAX-EO complex from 10 nsec intervals from 0-100 nsec molecular dynamics (MD) simulation. Residues of interest are represented as sticks for clarity. FIGS. 4b and 4c show distance relative to the time of EO carboxylate-R145 (carbonyl carbon-ζ-carbon) (FIG. 4b) and EO pyrazolone carbonyl-R134 (carbonyl oxygen-ζ-carbon) (FIG. 4c). Light shades represent individual MD simulation distances, and black represents the mean of n=3 simulations. FIGS. 4d and 4e show histogram representation of α-carbon distance frequency during MD simulation between R134 and negatively charged residues D48 (FIG. 4d) and E44 (FIG. 4e) on the α1-α2 loop. Data represent mean of n=3 for both BAX and BAX-EO complex. FIG. 4f shows a percentage change in root mean square fluctuation (RMSF) of BAX-EO versus BAX, plotted with respect to BAX residue number. Data represent mean of n=3 simulations for both BAX and BAX-EO. The average percentage change is represented by the dotted black line, with ±SD represented as dashed lines. FIG. 4g shows changes in structure and dynamics of α7/α4-α5 loop interface: representative α-carbon distance frequency histogram for F105-Q155 (left), transparent surface with ribbon representation of α7/α4-α5 loop interface with residues of note highlighted (center), and graphical representation of distances between residues at α7/α4-α5 loop interface (right). FIG. 4h shows changes in structure and dynamics of the canonical site opening formed by α3, loop 3, α4, and α9: representative α-carbon distance frequency histogram for T85-K189 (left), transparent surface with ribbon representation of canonical site opening with residues of note highlighted (center), and graphical representation of distances between residues at the canonical site opening (right). Distances represent the mean difference of n=3 BAX and BAX-EO MD simulations (FIGS. 4g and 4h), plotted with respect to time and distance frequency histograms for all distances are available in supplemental figures as referenced.

FIGS. 5a, 5b, 5c, 5d, and 5e are a set of diagrams showing NMR-based evidence of EO-mediated BAX inhibition. FIG. 5a shows EO-induced differences in peak intensity ratio (PRE) of peaks in the presence and absence of the soluble paramagnetic probe hydroxyl-TEMPO. Percentage change in PRE is plotted with respect to BAX residue number. Residues that exhibited increased or decreased PRE are labeled in shades of red and blue, respectively, the color gradient corresponding to the figure key. Residues associated with the N-terminal trigger site, BH3-domain, canonical site, and transmembrane domain are highlighted. FIG. 5b shows ribbon representation of BAX-EO complex with mapping of residues undergoing significant differences in PRE, as shown in FIG. 5a. FIG. 5c shows a ribbon representation of EO binding site with residues undergoing significant change in PRE colored corresponding to FIG. 5a and represented as a transparent surface with a stick representation of residues. FIGS. 5d and 5e show a cluster of residues about the α7/α4-α5 loop interface (FIG. 5d) and canonical site (FIG. 5e), which exhibited reduced PRE in the presence of EO.

FIGS. 6a, 6b, 6c, 6d, 6e, and 6f are a set of diagrams showing that EO inhibits BAX-mediated cell death. FIGS. 6a and 6b show percentage inhibition of cytochrome c release determined by ELISA in the presence of BIM-BH3 peptide and increasing doses of EO in BAKKO mouse embryonic fibroblasts (MEFs) (FIG. 6a). FIG. 6b shows the dose-response EC50 curve for percentage inhibition of BIM-BH3 induced cytochrome c release by EO with EC50 shown for clarity. Data represent mean of n=3±SEM and are representative of three independent experiments. FIG. 6c shows the results of the cellular thermal shift assay (CETSA) BAX melting curves for BAKKO MEFs treated with vehicle or 10 μM EO. Data represent mean of n=4±SEM independent experiments. FIG. 6d shows the results of the viability assay of 3T3 cells upon treatment with 1 μM ABT-263 (Navitoclax) and 1 μM S63845 in the presence or absence of various doses of EO for 24 hours. Viability upon ABT-263 and S63845 combination in the absence of EO is indicated by the dashed line. Data represent mean of n=3±SEM and are representative of three independent experiments. FIG. 6e shows the results of the caspase 3/7 assay of 3T3 cells upon treatment with 1 μM ABT-263 and 1 μM S63845 in the presence or absence of various doses of EO for 4 hours. Data represent mean of n=3±SEM and are representative of three independent experiments. Two-sided t-test, ****P<0.0001; ***P<0.001;**P<0.01; *P<0.05; ns, P>0.05. FIG. 6f shows BAX surface representations showing distinct binding and associated conformational changes by the BAX trigger site inhibitor, EO, and the BAX trigger site activator, BTSA1.

FIGS. 7a and 7b are a set of diagrams showing that EO and EO analogs bind to the BAX trigger site and inhibit tBID-induced BAX activity. FIG. 7a shows binding of EO and EO analogs to the BAX trigger site. The results of the competitive fluorescence polarization (FP) binding assay show competitive binding of EO, EO-1, and EO-2 compounds to the BAX trigger site. FIG. 7b shows tBID-induced BAX inhibition of EO and EO analogs. The results of the BAX-mediated membrane permeabilization assay using liposomes with 50 nM BAX and 5 nM tBID, each at 30 minutes, show that EO and EO analogs inhibit tBID-induced BAX activity.

FIGS. 8a and 8b are a set of graphs showing that eltrombopag inhibited BAX-mediated cell death. FIG. 8a shows platelet counts of C57BL/6J mice treated with vehicle, ABT-263 (25 mg/kg, single dose), EO (100 mg/kg, then 3 hrs later 50 mg/kg), or a combination of ABT-263 (or navitoclax) and EO by oral gavage. Data represent mean of n=5±SEM. FIG. 8b shows % platelet protection plotted based on the data in FIG. 8a.

FIGS. 9a, 9b, 9c, 9d, 9e, and 9f are a set of diagrams showing that eltrombopag inhibits BAX translocation and BAX-mediated apoptosis in cells. FIG. 9a shows the confocal micrographs of BAK KO MEFs treated with DMSO, 2 μM STS for 4.5 h without or with 10 μM EO 6.5 h, respectively. BAX translocation is based on antibody-based detection of BAX and mitochondrial protein TOMM20. Representative confocal micrographs from three independent biological experiments. Scale bar, 20 m. FIG. 9b shows quantification of BAX translocation (% of cells with BAX foci colocalizing with TOMM20 foci) in BAK KO MEFs induced by 2 μM staurosporine (STS) and inhibited by 10 μM EO. Data represent ±SEM of three independent biological replicates. Two-sided t test, ****P<0.0001; ***P<0.001; **P<0.01; *P<0.05; ns, P>0.05. FIG. 9c shows representative immunoblot analysis of BAX translocation in BAK KO MEF cells in response to BIM BH3 titration in the presence of 10 or 20 μM EO. Actin and VDAC are loading controls for cytosolic and mitochondrial fractions, respectively. FIGS. 9d and 9e show a caspase 3/7 assay of BAK−/− mouse embryonic fibroblasts (MEFs) (FIG. 9d) and BAX−/−MEFs (FIG. 9e) in response to 3 μM STS and the presence or absence of various doses of EO for 6 hr. Data represent mean of n=3±SEM and are representative of three independent experiments. Two-sided t-test, **** P<0.0001; ***P<0.001;**P<0.01; *P<0.05; ns, P>0.05. FIG. 9f shows apoptosis detection using RealTime Glo (Promega) assay in human cardiomyocytes cells (IPSC-CM) in response to 5 μM cardiotoxic Doxorubicin and the presence or absence of 10 μM EO at various time points. Data represent mean of n=3±SEM.

FIGS. 10a, 10b, 10c, 10d, 10e, and 10f are a set of graphs showing the results of a liposome release assay for inhibition of BAX activity and BAX-mediated membrane permeabilization with 50 nM BAX and 5 nM tBID by EO-5, EO-7, EO-8, EO-18, EO-24, and EO-25, respectively.

### DETAILED DESCRIPTION OF THE INVENTION

This disclosure provides novel compounds and methods for inhibiting BAX activity and BAX-mediated apoptosis, as well as compounds and methods for treating or preventing BAX-mediated disorders.

The compounds as disclosed herein were developed based, at least in part, on the structural model of EO with BAX using NMR and molecular dynamics methods. As demonstrated in this disclosure, EO unexpectedly inhibits BAX activation by a novel two-fold mechanism. BAX inhibition by EO was dependent on the concentration of EO, BAX, and BH3-activators, and EO directly engaged the BAX trigger site binding, consistent with a direct competitive mechanism. Furthermore, EO inhibited heat-induced translocation and activation of BAX, promoted stabilization of the α1-α2 loop in closed conformation and interaction with α6, and induced conformational changes associated with reduced BAX activity, such as those observed at the α7/α4-α5 loop and canonical site-α9 interfaces. Thus, this disclosure presents a unique mechanism of BAX inhibition by EO that directly competes with BH3-only proteins for binding to BAX and simultaneously promotes allosteric conformational changes that stabilize the inactive soluble BAX structure. EO engages the trigger site with a unique binding mode distinct from BAX activators, using hydrophobic interactions with a shallow hydrophobic groove formed by residues of α6, α1, and the closed α1-α2 loop. Thus, EO and the disclosed compounds represent a novel class of BAX inhibitors for inhibition of BAX activity and BAX-mediated cell death.

### A. Compounds and Compositions

In one aspect, this disclosure provides compounds or a stereoisomer thereof, a derivative/analog thereof, a prodrug thereof, a metabolite thereof, or a pharmaceutically acceptable salt thereof, or a pharmaceutically acceptable salt thereof that bind to a BAX protein and inhibit activation or function of the BAX protein.

In some embodiments, the compounds can be represented by Formula (I):

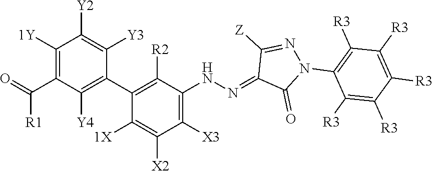

or
Formula (II):

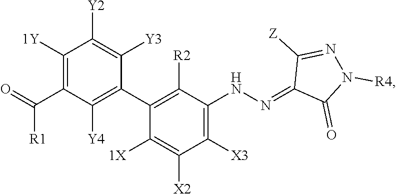

wherein:
wherein:
R1 is selected from OH, O—CH₃, O—CH₂CH₃, O—CH(CH₃)₂, NH—CH₃, and NH—CH₂CH₃;
R2 is selected from H, F, Cl, CH₃, CF₃, OCH₃, CH₂CH₃, OCH₂CH₃;
R3 is selected from H, OH, CH₃, CF₃, NH₂, F, Cl, OCH₃, and NHCOCH₃;
X1, X2, or X3 is selected from H, F, Cl, OH, CH₃, CF₃, CH₂CH₃, OCH₃, OCH₂CH₃, and —CO—CH₃;
Y1, Y2, Y3, or Y4 is selected from H, F, Cl, CH₃, CF₃, OCH₃, and CH₂CH₃;
Z is selected from H, F, Cl, CH₃, CF₃, OCH₃, CH₂CH₃, CH₂NH₂, and CH₂CH₂NH₂;
R4 is selected from:

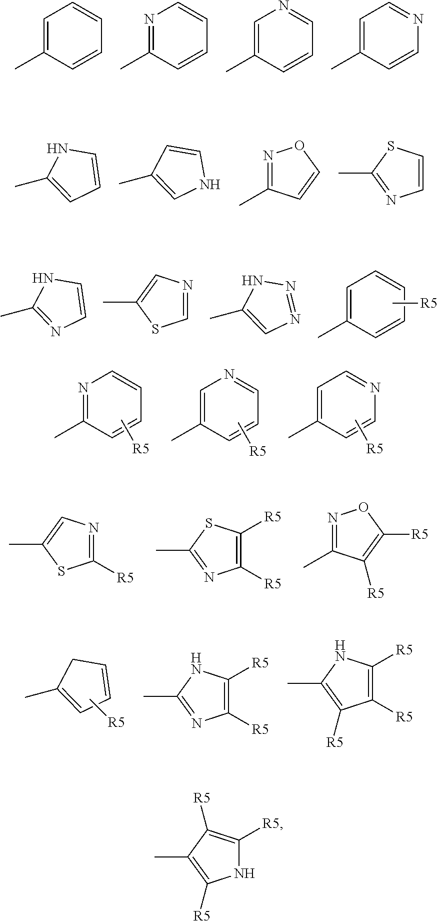

R5 is selected from H, OH, CH₃, CF₃, NH₂, F, Cl, OCH₃, and NHCOCH₃.

In some embodiments, representative compounds may include, without limitation,

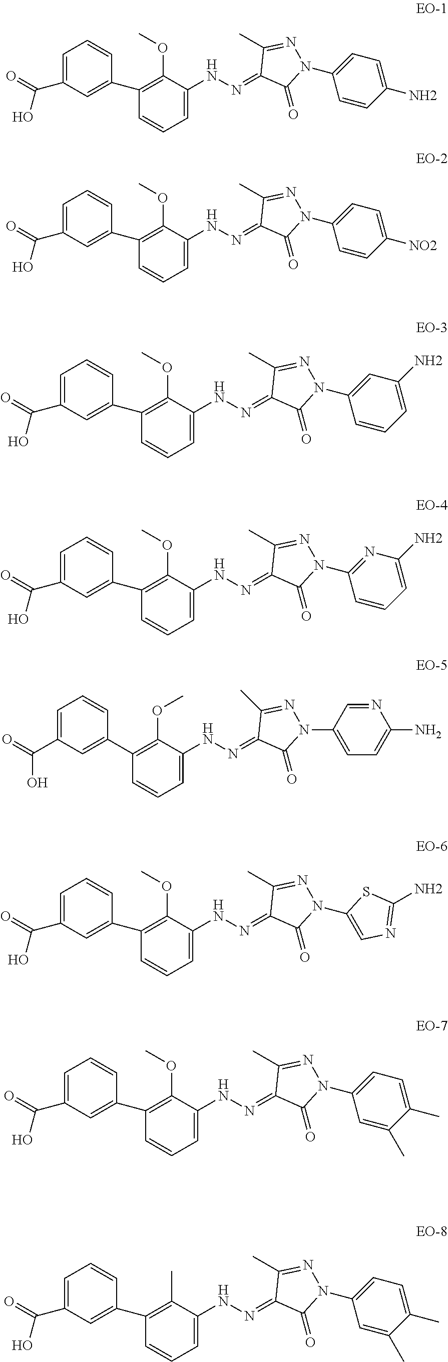

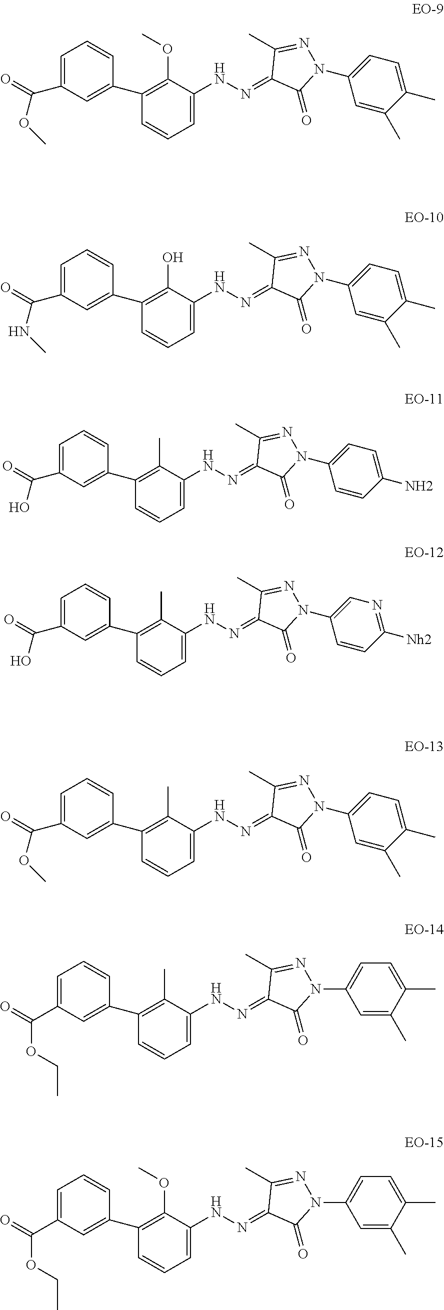

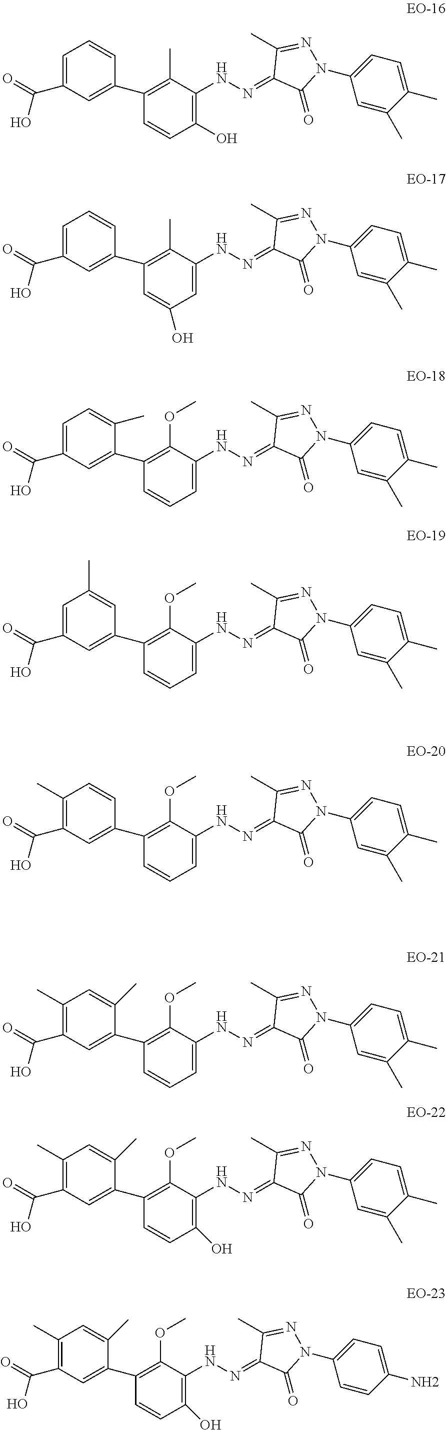

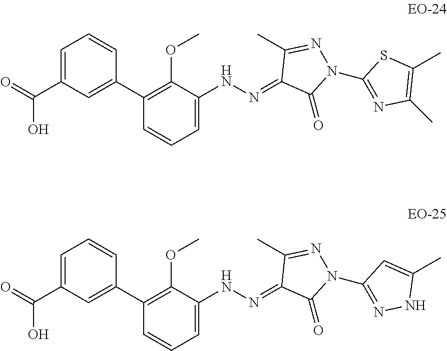 [SMILES: COc1c(N/N=C2/C(=O)N(c3nc(C)c(C)s3)N=C2C)cccc1-c1cccc(C(=O)O)c1 ; COc1c(N/N=C2/C(=O)N(c3cc(C)[nH]n3)N=C2C)cccc1-c1cccc(C(=O)O)c1]

Also provided in this disclosure is a pharmaceutical composition comprising (i) a compound described above, a stereoisomer thereof, a derivative/analog thereof, a prodrug thereof, a metabolite thereof, or a pharmaceutically acceptable salt thereof, and (ii) a pharmaceutically acceptable carrier.

In some embodiments, the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable.

Diastereomers are stereoisomers that are not related as mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms. The symbol “*” in a structural formula represents the presence of a chiral carbon center.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “St,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by a structure without indicating the stereochemistry, and the compound has at least one chiral center, it is to be understood that the name or structure encompasses one enantiomer of compound free from the corresponding optical isomer, a racemic mixture of the compound and mixtures enriched in one enantiomer relative to its corresponding optical isomer.

When a disclosed compound is named or depicted by a structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound. “Pharmaceutically active derivative” refers to any compound that, upon administration to the recipient, is capable of providing directly or indirectly, the activity disclosed herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds (e.g., compounds of Formula (I) or Formula (II)) can form salts which are also within the scope of this disclosure. Unless otherwise indicated, reference to an inventive compound is understood to include reference to one or more salts thereof. The term “salt(s)” denotes acidic and/or basic salts formed with inorganic and/or organic acids and bases. In addition, the term “salt(s) may include zwitterions (inner salts), e.g., when a compound contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the disclosure. Salts of the compounds may be formed, for example, by reacting a compound with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as, olamine, trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long-chain halides (e.g., decyl, lauryl, myristyl, and stearyl chlorides, bromides and iodides), aralkyl halides (e.g., benzyl and phenethyl bromides), and others. In some embodiments, examples of salts include monohydrochloride, hydrogensulfate, methanesulfonate, phosphate or nitrate salts.

Various forms of prodrugs are well known in the art and are described in: (a) The Practice of Medicinal Chemistry, Camille G. Wermuth et al., Ch 31, (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds. Ch 5, pgs 113-191 (Harwood Academic Publishers, 1991); and (d) Hydrolysis in Drug and Prodrug Metabolism, Bernard Testa and Joachim M. Mayer, (Wiley-VCH, 2003).

In addition, the compounds, subsequent to their preparation, can be isolated and purified to obtain a composition containing an amount by weight equal to or greater than 99% of a compound (“substantially pure”), which is then used or formulated as described herein. Such “substantially pure” compounds are also contemplated herein as part of the present disclosure. “Stable compound” and “stable structure” are meant to indicate a compound that is sufficiently robust to survive isolation to a useful degree of purity from a reaction mixture, and formulation into an efficacious therapeutic agent. The present disclosure is intended to embody stable compounds.

The compounds of the present disclosure are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as —CD₃.

Compounds and/or pharmaceutically acceptable salts thereof can be administered by any means suitable for the condition to be treated, which can depend on the need for site-specific treatment or quantity of the compound to be delivered. Also embraced within this disclosure is a class of pharmaceutical compositions comprising a compound and/or pharmaceutically acceptable salts thereof, and one or more non-toxic, pharmaceutically-acceptable carriers and/or diluents and/or adjuvants (collectively referred to herein as “carrier” materials) and, if desired, other active ingredients. The compounds may be administered by any suitable route, preferably in the form of a pharmaceutical composition adapted to such a route, and in a dose effective for the treatment intended. The compounds and compositions of the present disclosure may, for example, be administered orally, mucosally, rectally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g., magnesium stearate, and a disintegrating agent such as crospovidone. The carrier mixture may be filled into a gelatin capsule or compressed as a tablet. The pharmaceutical composition may be administered as an oral dosage form or an infusion, for example.

Techniques and formulations generally may be found in Remmington's Pharmaceutical Sciences, Meade Publishing Co., Easton, PA. For systemic administration, injection is preferred, including intramuscular, intravenous, intraperitoneal, and subcutaneous. For injection, the agents can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but can be determined using routine methods.

Any pharmaceutical composition contemplated herein can, for example, be delivered orally via any acceptable and suitable oral preparations. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium croscarmellose, corn starch, and alginic acid; binding agents, such as, for example, starch, gelatin, polyvinylpyrrolidone, and acacia; and lubricating agents, such as, for example, magnesium stearate, stearic acid, and talc. Additionally, a tablet can either be uncoated or coated by known techniques to either mask the bad taste of an unpleasant tasting drug or delay disintegration and absorption of the active ingredient in the gastrointestinal tract, thereby sustaining the effects of the active ingredient for a longer period. Exemplary water-soluble taste-masking materials include, but are not limited to, hydroxypropyl-methylcellulose and hydroxypropyl-cellulose. Exemplary time delay materials include, but are not limited to, ethylcellulose and cellulose acetate butyrate.

Hard gelatin capsules can, for example, be prepared by mixing at least one compound and/or at least one salt thereof with at least one inert solid diluent, such as, for example, calcium carbonate; calcium phosphate; and kaolin.

Soft gelatin capsules can, for example, be prepared by mixing at least one compound and/or at least one pharmaceutically acceptable salt thereof with at least one water-soluble carrier, such as, for example, polyethylene glycol; and at least one oil medium, such as, for example, peanut oil, liquid paraffin, and olive oil.

An aqueous suspension can be prepared, for example, by admixing at least one compound and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethylcellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long-chain aliphatic alcohols, such as, for example, heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound and/or at least one pharmaceutically acceptable salt thereof in either vegetable oil, such as, for example, Arachis oil; olive oil; sesame oil; and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisole, and alpha-tocopherol.

Dispersible powders and granules can, for example, be prepared by admixing at least one compound and/or at least one pharmaceutically acceptable salt thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

An emulsion of at least one compound and/or at least one pharmaceutically acceptable salt thereof can, for example, be prepared as an oil-in-water emulsion. The oily phase of the emulsions comprising compounds may be constituted from known ingredients in a known manner. The oil phase can be provided by, but is not limited to, for example, a vegetable oil, such as, for example, olive oil, Arachis oil, a mineral oil, such as, for example, liquid paraffin, and mixtures thereof. While the phase may comprise merely an emulsifier, it may comprise a mixture of at least one emulsifier with a fat or an oil or with both fat and oil. Suitable emulsifying agents include, but are not limited to, for example, naturally-occurring phosphatides, e.g., soybean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. In some embodiments, a hydrophilic emulsifier is included together with a lipophilic emulsifier, which acts as a stabilizer. It is also preferred to include both oil and fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceryl distearate alone or with a wax, or other materials well known in the art.

A pharmaceutical composition described herein can also be incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier does not adversely affect the active agent or other components of the topical formulation. Examples of suitable topical carriers for use herein include water, alcohols, and other nontoxic organic solvents, glycerin, mineral oil, silicone, petroleum jelly, lanolin, fatty acids, vegetable oils, parabens, waxes, and the like.

Pharmaceutical compositions may be incorporated into gel formulations, which generally are semisolid systems consisting of either suspension made up of small inorganic particles (two-phase systems) or large organic molecules distributed substantially uniformly throughout a carrier liquid (single-phase gels). Single-phase gels can be made, for example, by combining the active agent, a carrier liquid, and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose, and gelatin. Although gels commonly employ aqueous carrier liquid, alcohols and oils can be used as the carrier liquid as well.

The compounds and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e., Captisol), cosolvent solubilization (i.e., propylene glycol) or micellar solubilization (i.e., Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

A sterile injectable oil-in-water microemulsion can, for example, be prepared by (1) dissolving at least one compound in an oily phase, such as, for example, a mixture of soybean oil and lecithin; (2) combining containing oil phase with a water and glycerol mixture; and (3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butanediol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this disclosure include, but are not limited to, ion exchangers, alumina, aluminum stearate, lecithin, self-emulsifying drug delivery systems (SEDDS) such as alpha-tocopherol polyethylene glycol 1000 succinate, surfactants used in pharmaceutical dosage forms such as Tweens, polyethoxylated castor oil, such as cremophor surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this disclosure can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as additives, preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients, and preservatives. An optimum topical formulation comprises approximately: 2 wt. % to 60 wt. %, preferably 2 wt. % to 50 wt. %, solubilizer and/or skin permeation enhancer; 2 wt. % to 50 wt. % preferably 2 wt. % to 20 wt. % emulsifiers; 2 wt. % to 20 wt. % emollient; and 0.01 to 0.2 wt. % preservative, with the active agent and carrier (e.g., water) making of the remainder of the formulation. A skin permeation enhancer serves to facilitate passage of therapeutic levels of active agent to pass through a reasonably sized area of unbroken skin. Suitable enhancers are well known in the art and include, for example, lower alkanols such as methanol ethanol and 2-propanol; alkyl methyl sulfoxides such as dimethylsulfoxide (DMSO), decylmethylsulfoxide (C.sub.lO MSO) and tetradecylmethyl sulfoxide; pyrrolidones such as 2-pyrrolidone, N-methyl-2-pyrrolidone and N-(-hydroxyethyl)pyrrolidone; urea; N,N-diethyl-m-toluamide; C.sub.2-C.sub.6 alkane diols; miscellaneous solvents such as dimethylformamide (DMF), N,N-dimethylacetamide (DMA), and tetrahydrofurfuryl alcohol; and the 1-substituted azacycloheptan-2-ones, particularly 1-n-dodecylcyclazacycloheptan-2-one (laurocapram; available under the trademark Azone® from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate). In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation. Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition.

The amounts of compounds that are administered and the dosage regimen for treating a disease condition with the compounds and/or compositions of this disclosure depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two-day cycle.

For therapeutic purposes, the active compounds of this disclosure are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethylcellulose.

Pharmaceutical compositions of this disclosure comprise at least one compound and/or at least one pharmaceutically acceptable salt thereof, and optionally an additional agent selected from any pharmaceutically acceptable carrier, adjuvant, and vehicle. Alternate compositions of this disclosure comprise a compound described herein, or a prodrug thereof, and a pharmaceutically acceptable carrier, adjuvant, or vehicle.

The compound, the composition or the pharmaceutical composition described herein can be provided in a kit. In one embodiment, the kit includes (a) a container that contains the composition and optionally (b) informational material. The informational material can be descriptive, instructional, marketing or other material that relates to the methods described herein and/or the use of the agents for therapeutic benefit. For example, kits may include instruction for the manufacturing, for the therapeutic regimen to be used, and periods of administration. In an embodiment, the kit includes also includes an additional therapeutic agent. The kit may comprise one or more containers, each with a different reagent. For example, the kit includes a first container that contains the composition and a second container for the additional therapeutic agent.

The containers can include a unit dosage of the pharmaceutical composition. In addition to the composition, the kit can include other ingredients, such as a solvent or buffer, an adjuvant, a stabilizer, or a preservative. The kit optionally includes a device suitable for administration of the composition, e.g., a syringe or other suitable delivery device. The device can be provided pre-loaded with one or both of the agents or can be empty, but suitable for loading.

### B. Methods of Use

In another aspect, this disclosure also provides a method of treating or preventing a disorder mediated by BAX in a subject. The method comprises administering to the subject a therapeutically effective amount of a compound or a stereoisomer or a pharmaceutically acceptable salt thereof, or a pharmaceutical composition, as described above. In some embodiments, the subject is a mammal, e.g., a human.

In some embodiments, the disorder is associated with increased expression or activation of the BAX protein. In some embodiments, the disorder comprises a neuronal disorder or an autoimmune disease. In some embodiments, the neuronal disorder is selected from epilepsy, multiple sclerosis, Alzheimer's disease, Huntington's disease, Parkinson's disease, retinal diseases, spinal cord injury, Crohn's disease, head trauma, spinocerebellar ataxias, and dentatorubral-pallidoluysian atrophy. In some embodiments, the autoimmune disease is selected from Multiple Sclerosis, amyotrophic lateral sclerosis, retinitis pigmentosa, inflammatory bowel disease (IBD), rheumatoid arthritis, asthma, septic shock, transplant rejection, and AIDS.

In some embodiments, the disorder comprises ischemia (e.g., stroke, myocardial infarction, and reperfusion injury), cardiomyopathy, chemotherapy-induced cardiotoxicity, chemotherapy-induced cardiomyopathy, cardiovascular disorders, arteriosclerosis, heart failure, viral infection heart injury, myocarditis, viral infection lung injury, heart transplantation, renal hypoxia, a liver disease, a kidney disease, an intestinal disease, liver ischemia, intestinal ischemia, acute optic nerve damage, glaucoma, Idiopathic pulmonary fibrosis (IPF), chemotherapy-induced ocular toxicity, hepatitis, viral infection.

As used herein, the term “administering” refers to the delivery of cells by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration.

As used herein, “treatment” or “treating,” or “palliating” or “ameliorating” are used interchangeably. These terms refer to an approach for obtaining beneficial or desired results, including, but not limited to, a therapeutic benefit and/or a prophylactic benefit. By therapeutic benefit is meant any therapeutically relevant improvement in or effect on one or more diseases (e.g., inflammatory diseases, neurodegenerative diseases, cardiovascular diseases), conditions, or symptoms under treatment. For prophylactic benefit, the agent or the compositions thereof may be administered to a subject at risk of developing a particular disease, condition, or symptom, or to a subject reporting one or more of the physiological symptoms of a disease, even though the disease, condition, or symptom may not have yet been manifested.

The terms “inhibit,” “decrease,” “reduced,” “reduction,” or “decrease” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

The terms “activate,” “increased,” “increase” or “enhance” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” is meant to refer to any change in biological state, i.e., increasing, decreasing, and the like.

Through the modulation of the BAX function, disorders mediated by BAX can be treated or prevented. Such disorders may include neuronal disorders and/or disorders of the immune system. The modulation may involve the inhibition of the activity (activation) and/or of the expression of BAX. For example, the modulation of the BAX function or activity may include the inhibition or disruption of the interaction of Bim, Bid, Bmf, Puma, or Noxa with BAX, which has been shown to play a role within the context of the BAX activation leading to cytochrome c release (J. C. Martinou et al. The Journal of Cell Biology, 144(5), 891-901 (1999)). As a result of the inhibition of the BAX activation by Bim, Bid, Bmf, Puma, or Noxa upon using the compounds described herein, the cytochrome c release could be inhibited or essentially blocked, thus providing a means to modulate the apoptosis pathways. As a result, by modulation of the apoptosis pathways, a wide variety of disorders associated with abnormal apoptosis can be treated.

In some embodiments, the compounds described herein are suitable for use in treating disorders associated with an abnormal BAX function or abnormal (e.g., elevated) BAX activation, an abnormal expression or activity of BAX. Thus, the treatment or prevention of disorders involves modulation (e.g., inhibition, disruption) of the BAX function or activation, in particular with the abnormal expression or activity of BAX, using the compounds described herein, a stereoisomer thereof, a derivative/analog thereof, a prodrug thereof, a metabolite thereof, or a pharmaceutically acceptable salt thereof.

For example, the compounds can be used for treating the disorders, such as neuronal disorders, autoimmune diseases, and cardiovascular diseases. In some embodiments, the neuronal disorder includes epilepsy, Alzheimer's disease, Huntington's disease, Parkinson's disease, retinal diseases, spinal cord injury, Crohn's disease, head trauma, spinocerebellar ataxias, multiple sclerosis, and dentatorubral-pallidoluysian atrophy.

In some embodiments, the autoimmune disease includes multiple sclerosis, amyotrophic lateral sclerosis, retinitis pigmentosa, inflammatory bowel disease (IBD), rheumatoid arthritis, asthma, septic shock, transplant rejection, and AIDS.

In some embodiments, the disorder comprises ischemia (e.g., stroke, myocardial infarction, and reperfusion injury), cardiomyopathy, chemotherapy-induced cardiotoxicity, chemotherapy-induced cardiomyopathy, cardiovascular disorders, arteriosclerosis, heart failure, viral infection heart injury, myocarditis, viral infection lung injury, heart transplantation, renal hypoxia, a liver disease, a kidney disease, an intestinal disease, liver ischemia, intestinal ischemia, acute optic nerve damage, glaucoma, Idiopathic pulmonary fibrosis (IPF), chemotherapy-induced ocular toxicity, hepatitis, viral infection.

The term “disease” as used herein is intended to be generally synonymous and is used interchangeably with, the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., neuronal disorders, autoimmune diseases, and cardiovascular diseases), and/or who has been diagnosed with inflammatory disorders. In some embodiments, the subject is a mammal. In some embodiments, the subject is human.

In some embodiments, the method further comprises administering to the subject a second therapeutic agent or therapy. In some embodiments, the second therapeutic agent comprises an anti-inflammatory agent or an anti-tumor/anti-cancer agent. In some embodiments, the anti-tumor/anti-cancer agent is navitoclax.

In some embodiments, the second therapeutic agent is administered to the subject before, after, or concurrently with the compounds described herein, a stereoisomer thereof, a derivative/analog thereof, a prodrug thereof, a metabolite thereof, or a pharmaceutically acceptable salt thereof.

In some embodiments, the subject was previously administered an anti-cancer therapy. In some embodiments, the anti-cancer therapy comprises surgery, radiation, chemotherapy, and/or immunotherapy. In some embodiments, the chemotherapy comprises a therapeutic agent that inhibits Bcl-xL. In some embodiments, the therapeutic agent that inhibits Bc-xL is navitoclax (or ABT-263).

Navitoclax is an orally active, small synthetic molecule and an antagonist of a subset of the B-cell leukemia 2 (Bcl-2) family of proteins with potential antineoplastic activity. Navitoclax selectively binds to apoptosis suppressor proteins Bcl-2, Bcl-xL, and Bcl-w, which are frequently overexpressed in a wide variety of cancers, including those of the lymph, breast, lung, prostate, and colon, and are linked to tumor drug resistance. Inhibition of these apoptosis suppressors prevents their binding to the apoptotic effectors BAX and BAK proteins, thereby triggering apoptotic processes in cells overexpressing Bcl-2, Bcl-xL, and Bcl-w. This eventually reduces tumor cell proliferation. Navitoclax has been used in trials studying the treatment of solid tumors, Non-Hodgkin's lymphoma, EGFR activating mutation, chronic lymphoid leukemia, hematological malignancies, and other cancers. The chemical structure of navitoclax is represented by the following formula:

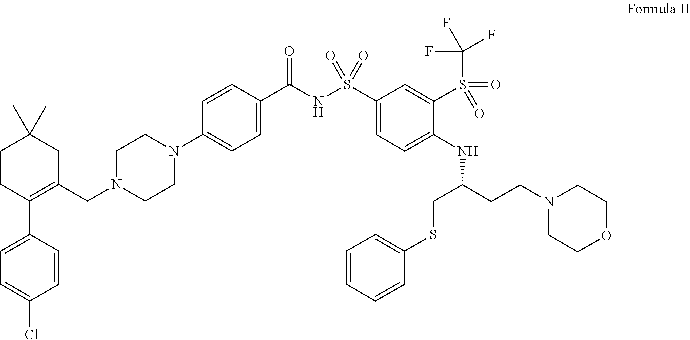 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, the compounds described herein, a stereoisomer thereof, a derivative/analog thereof, a prodrug thereof, a metabolite thereof, or a pharmaceutically acceptable salt thereof, is administered intratumorally, intravenously, subcutaneously, intraosseously, orally, transdermally, in sustained release, in controlled release, in delayed release, as a suppository, or sublingually.

In some embodiments, the compounds described herein, a stereoisomer thereof, a derivative/analog thereof, a prodrug thereof, a metabolite thereof, or a pharmaceutically acceptable salt thereof, is administered prophylactically or therapeutically.

In another aspect, this disclosure also provides a method of treating or ameliorating a symptom (e.g., platelet loss) of thrombocytopenia associated with treatment targeting Bcl-xL. The method comprises: (i) selecting a subject having a condition treatable by a therapeutic agent that inhibits Bcl-xL; and (ii) administering to the subject a therapeutically effective amount of the compounds described herein, a stereoisomer thereof, a derivative/analog thereof, a prodrug thereof, a metabolite thereof, or a pharmaceutically acceptable salt thereof, in combination with a therapeutically effective amount of the therapeutic agent.

In some embodiments, the condition is a cancer (e.g., solid tumors, Non-Hodgkin's lymphoma, EGFR activating mutation, chronic lymphoid leukemia, and hematological malignancies). In some embodiments, cancers are characterized by overexpression of a Bcl-2 family protein, such as lymphoma, skin cancer, pancreatic cancer, colon cancer, melanoma, liver cancer, bladder cancer, non-small cell lung cancer, myeloma, leukemia, and head and neck cancer.

In some embodiments, the condition is an autoimmune disease. Examples of autoimmune diseases may include multiple sclerosis, amyotrophic lateral sclerosis, retinitis pigmentosa, inflammatory bowel disease (IBD), rheumatoid arthritis, asthma, septic shock, transplant rejection, and AIDS.

In some embodiments, the therapeutic agent that inhibits Bcl-xL is navitoclax. In some embodiments, the therapeutic agent is administered to the subject before, after, or concurrently with the compounds described herein, a stereoisomer thereof, a derivative/analog thereof, a prodrug thereof, a metabolite thereof, or a pharmaceutically acceptable salt thereof.

In some embodiments, the therapeutic agent or the compounds described herein, a stereoisomer thereof, a derivative/analog thereof, a prodrug thereof, a metabolite thereof, or a pharmaceutically acceptable salt thereof, is administered to the subject in one or more doses.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In another aspect, this disclosure also provides a method of inhibiting BAX-mediated apoptosis in a cell (e.g., a neuronal cell, a cardiac cell). The method comprises administering to the cell expressing a BAX protein an effective amount of a compound described herein, a stereoisomer thereof, a derivative/analog thereof, a prodrug thereof, a metabolite thereof, or a pharmaceutically acceptable salt thereof that binds to the BAX protein and inhibits activation or function of the BAX protein.

“Apoptosis” refers to the process by which cells are programmed to die or lose viability. Commonly triggered by cytochrome leakage from the mitochondria and accompanied by signaling cascades (caspases and other proteins) resulting in decreased mitochondrial and energy potential via the electron transport system, a build-up of reactive oxygen species and free radical, and loss of membrane integrity.

In yet another aspect, this disclosure further provides a method of inhibiting activation or function of a BAX protein in a cell. The method comprises administering to the cell (e.g., a neuronal cell, a cardiac cell) expressing a BAX protein an effective amount of a compound described herein, a stereoisomer thereof, a derivative/analog thereof, a prodrug thereof, a metabolite thereof, or a pharmaceutically acceptable salt thereof, that binds to the BAX protein.

In some embodiments, the method comprises inhibiting the activation of BAX protein that is mediated by Bim, Bid, Bmf, Puma, or Noxa.

In another aspect, this disclosure provides a method for preserving or treating an organ or tissue. The method comprises contacting the organ or tissue with an effective amount of a compound or a stereoisomer or a pharmaceutically acceptable salt thereof as described herein, under conditions effective for preservation of the organ or tissue.

### C. Definitions

To aid in understanding the detailed description of the compositions and methods according to the disclosure, a few express definitions are provided to facilitate an unambiguous disclosure of the various aspects of the disclosure. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means the amount of such a substance that produces some desired local or systemic effect at a reasonable benefit/risk ratio applicable to any treatment. The therapeutically effective amount of such substance will vary depending upon the subject and disease or condition being treated, the weight and age of the subject, the severity of the disease or condition, the manner of administration, and the like, which can readily be determined by one of ordinary skill in the art. For example, certain compositions described herein may be administered in a sufficient amount to produce a desired effect at a reasonable benefit/risk ratio applicable to such treatment.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product(s).” If the polynucleotide is derived from genomic DNA, expression may include splicing of the mRNA in a eukaryotic cell.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the invention and are physiologically acceptable to the subject. Supplementary active compounds may also be incorporated into the compositions.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

As used herein, the term “each,” when used in reference to a collection of items, is intended to identify an individual item in the collection but does not necessarily refer to every item in the collection. Exceptions can occur 1f explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. Examples

### Example 1

This example describes the materials and methods used in subsequent EXAMPLES below.

### Reagents

Hydrocarbon-stapled peptides corresponding to the BH3 domain of BIM, BIM SAHB: FITC-Ahx-EIWIAQELRS5IGDS5FNAYYA-CONH (SEQ ID NO: 1), where S5 represents the non-natural amino acid inserted for olefin metathesis, were synthesized, purified at >95% purity by CPC Scientific Inc. and characterized as previously described (Gavathiotis, E., et al. Nat Chem Biol 8, 639-645 (2012)). Peptides corresponding to the BH3-domain of BIM, BIM-BH3, Ac-RPEIWIAQELRRIGDEFNAYYARR (SEQ ID NO: 2), was synthesized by GenScript at >95% purity. Recombinant tBID in >95% purity by SDS-PAGE under reducing conditions was purchased by R&D Systems (cat. #882-B8-050). Eltrombopag (EO) (cat. #100941) and eltrombopag methyl ester (cat. #SC498745) were purchased from Medkoo Biosciences, and Santa Cruz Biotechnology, respectively, and their molecular identity and purity>95% was confirmed by NMR. ABT-263 (cat. #S1001), and S63845 (cat. #A8737) were purchased from Selleckchem, and APExBIO, respectively. Compounds were stored as powdered, reconstituted into 100% DMSO and diluted as described.

### Production of Recombinant BAX

Human full-length (1-192) wild-type BAX (Q07812; SEQ ID NO: 3) was cloned in pTYB1 vector (New England BioLabs) between the NdeI and SapI restriction sites. Mutations were generated using the QuickChange Lightning site-directed mutagenesis kit (Agilent). Recombinant proteins were expressed in BL21 (DE3) CodonPlus (DE3)-RIPL, grown in Luria Broth media and induced with 1 mM isopropyl β-d-1-thiogalactopyranoside (IPTG). The bacterial pellet was resuspended in lysis buffer (20 mM Tris HCl pH 7.6, 250 mM NaCl, 1 mM EDTA, and Roche complete EDTA free protease inhibitor cocktail), lysed by high-pressure homogenization, and clarified by ultracentrifugation at 45,000×g for 45 min. The supernatant was applied to 5 ml of pre-equilibrated chitin beads (New England BioLabs) in a gravity-flow column and washed with 3 column volumes of lysis buffer. BAX was cleaved by overnight incubation using 50 mM DTT in lysis buffer. Cleaved BAX was eluted with lysis buffer, concentrated with a Centricon spin concentrator (Millipore) and purified by gel filtration using a Superdex 75 10/300 GL column (GE Healthcare Life Sciences), pre-equilibrated with gel filtration buffer (20 mM HEPES, 150 mM KCl, pH 7.2) at 4° C. Fractions containing BAX monomer were pooled and concentrated using a 10-KDa cut-off Centricon spin concentrator (Millipore) for prompt use in biochemical and structural studies.

### Fluorescence Polarization Binding Assays

Fluorescence polarization assays (FPA) were performed as previously described (Reyna, D. E., et al. Cancer Cell 32 490-505 (2017)). Direct binding isotherms of BIM-SAHB were measured by incubated FITC-BIM-SAHB (25 nM) with serial dilutions of full-length BAX alone or in the presence of 0.5 or 1 μM EO. Competition binding assays were performed by titrating EO into BAX (150 nM) and FITC-BIM-SAHB (25 nM). Measurements were taken at 10-minute intervals over 60 minutes on a TECAN F200 PRO microplate reader. Reported curves represent a 10-minute time point. KD values and IC50 were determined using GraphPad Prism nonlinear fit four-parameter agonist or antagonist versus response with restraints for 100% and 0% bound calculated by the mP of saturated BAX+FITC-BIM-SAHB and FITC-BIM-SAHB alone.

### Microscale Thermophoresis

Recombinant BAX C62S C126S S5C (4C), previously established for evaluating BAX binding compounds with MST (Garner, T. P., et al. Nat Chem Biol 15, 322-330 (2019)), or BAX C62S C126S S5C R134E R145E (R134E R145E 4C) was labeled at cysteine using the Monolith Protein Labeling Kit Red Maleimide (NanoTemper Technologies) according to the instructions of the manufacturer (Amgalan D, et al. Nature Cancer 1, 315-328(2020)). Briefly, 10 μM protein was incubated with 0.9 equivalents of dye in MST buffer (100 mM potassium phosphate, pH 7.4, 150 mM NaCl) in the dark at room temperature (22-25° C.) for 1 hour. Unreacted dye was quenched using 5 mM DTT and removed using the manufacturer provided buffer exchange column. To determine the KD of BAX to EO, 50 nM labeled BAX was incubated with increasing concentrations of EO in MST buffer supplemented with 0.25% CHAPS. Samples were loaded into standard glass capillaries (Monolith NT.155 Capillaries) and analyzed by MST using a Monolith NT.115 Blue/Red, LED power and IR laser power of 80%. Fraction bound and error was generated by NanoTemper software (MO.Affinity Analysis), and KD values were determined using GraphPad Prism nonlinear fit four-parameter agonist versus response with restraints for 0 and 1 fraction bound.

### Liposomal Permeabilization Assay

Lipids (Avanti Polar Lipids) at the following ratio, phosphatidylcholine 48%, phosphatidylinositol 10%, dioleoyl phosphatidylserine 10%, phosphatidylethanolamine, 28%, and tetraoleoyl cardiolipin 4%, were mixed in a total of 1 mg, dried and resuspended in 10 mM HEPES, pH 7, 200 mM KCl, and 5 mM MgCl2 with 12.5 mM 8-aminonaphthalene-1,3,6-trisulfonic acid (ANTS) dye and 45 mM p-xylene-bis-pyridinium bromide (DPX) quencher (Molecular Probes) using a water bath sonicator. Liposomes were formed by extrusion of the suspension using Avanti Mini-Extruder (cat #610000) with polycarbonate membranes of 0.1 μm pore size (Avanti Polar Lipids). ANTS/DPX encapsulated liposomes were purified from non-encapsulated ANTS/DPX by gel filtration of a 10 mL CL2B-Sepharose (GE Healthcare Life Sciences) gravity flow column. BAX (50-250 nM) was combined with tBID, BIM, and EO at the indicated concentrations to a volume of 90 μL. Reactions were initiated by the addition of 10 μL of the encapsulated ANTS/DPX liposome stock. ANTS/DPX release was quantified based on the increase in fluorescence intensity that occurs when the ANTS fluorophore is separated from the DPX quencher upon release from the liposomes into solution. Fluorescence (λex=355 nm and λem=520 nm) was measured at 1-minute intervals at room temperature (22-25° C.) indicated using a Tecan Infinite M1000 plate reader. In the case of heat activation, reactions were setup as described in the absence of tBID or BIM, and experiments were recorded at 42° C. The percentage release of ANTS/DPX at any given time point was calculated as percentage release=((F−F0)/(F100−F0))(100), where F0 and F100 are baseline and maximal fluorescence, respectively. Triton X-100 (1%) was used to determine the maximum amount of liposomal release per assay and was set to 100%.

### Liposomal Translocation Assay

Lipids (Avanti Polar Lipids) at the following ratio, phosphatidylcholine 48%, phosphatidylinositol 10%, dioleoyl phosphatidylserine 10%, phosphatidylethanolamine, 28%, and tetraoleoyl cardiolipin 4%, were mixed in a total of 1 mg, dried and resuspended in 10 mM HEPES, pH 7, 200 mM KCl, and 5 mM MgCl2. The resulting slurry was vortexed for 10 minutes and sonicated in a sonicating water bath for 10 minutes. Liposomes were formed by extrusion of the suspension using Avanti Mini-Extruder with polycarbonate membranes of 0.1 μm pore size (Avanti Polar Lipids) followed by passage through a CL2B Sepharose column (GE Healthcare). Recombinant wild type BAX was labeled at cysteine by overnight incubation at 4° C. with 10 equivalents of iodoamino-NBD (IANBD, Thermo Fisher) and 3 equivalents of TCEP to maintain reduced cysteine. Labeled BAX (BAX-NBD) was separated from unreacted IANBD by gel filtration (Econo-Pac 10 DG desalting column, BioRad) and used immediately. Translocation reactions were performed by combining 800 nM BAX with 1 μM BIM or 200 nM tBID in the presence and absence of varying doses of EO. Reactions were initiated by the addition of 10 μL of the liposome stock. The NBD fluorophore exhibits low fluorescence in solution due to quenching by water. Upon BAX-NBD translocation, the NBD fluorophore is excluded from bulk water through contact with the liposomal membrane leading to an increase in fluorescence intensity. Fluorescence (λex=475 nm and λem=530 nm) was measured at 1-minute intervals at 37° C. indicated using a Tecan Infinite M1000 plate reader. The percentage translocation at any given time point was calculated as percentage translocation=[((F−F0)/(F100−F0))(100)]−[((FS−FS0)/(FS100−FS0))(100)] where F0 and F100 are baselines, and maximal fluorescence, respectively and FS, FS0, and FS100 are the current fluorescence, baseline fluorescence, maximal fluorescence of solution BAX incubated in the absence of liposomes. The subtraction of the percent translocation of solution BAX is required to correct for NBD fluorescence bleaching that occurs throughout the reaction. Triton X-100 (0.1%) was used to determine the maximum amount of liposomal translocation per assay and was set to F100 100%.

### BAX Conformation Change Assay Using Anti-6A7 Immunoprecipitation

Exposure of the 6A7 epitope of BAX was assessed by immunoprecipitation with a 6A7-domain-specific antibody purchased from Santa Cruz (SC-23959). Protein G beads (50 μL, Santa Cruz) were washed three times with 3% BSA in PBS and incubated with 15 μL 6A7 antibody at 4° C. for 1 hour. Recombinant full-length BAX (10 μM) was incubated with 4 equivalents of BIM-BH3 peptide alone and in the presence of 5 or 10 equivalents of EO for 15 min at room temperature. Incubation of full-length recombinant BAX with 0.1% Triton X served as a positive control for exposure of the 6A7 epitope. After incubation, 10 μL of each reaction was transferred to the protein G beads pre-loaded with an anti-6A7 antibody, and 1 μL was reserved as a loading control. After 90 minutes of incubation at 4° C., beads were collected and washed three times with 500 μL of 3% BSA in PBS and solubilized with 25 μL LDS/DTT loading buffer. Samples were resolved by SDS-PAGE electrophoresis and western blot analysis with an anti-BAX YTH-2D2 antibody (Invitrogen).

### Western Blotting and Protein Quantification

BAX samples were electrophoretically separated on 4-12% NuPage (Invitrogen) gels, transferred to mobilon-FL PVDF membranes (Millipore) and subjected to immunoblotting. For visualization of proteins with Odyssey Infrared Imaging System (LI-COR Biosciences), membranes were blocked in PBS containing 2.5% milk powder. Primary BAX YTH-2d2 antibody (R&D Systems, cat. #2282-MC-100) was incubated overnight at 4° C. in a 1:1,000 dilution. After washing, membranes were incubated with an IRdye800-conjugated goat anti-mouse IgG secondary antibody (LI-COR Biosciences, cat. #926-68022) in a 1:5,000 dilution. Protein was detected with the Odyssey Infrared Imaging System. Densitometry of protein bands was acquired using an LI-COR Odyssey scanner. Quantification and analysis were performed using the Western Analysis tool from the Image Studio 3.1 software.

### NMR Samples and Spectroscopy

The uniformly ¹⁵N-labeled protein samples were prepared by growing the bacteria in the minimal medium, as previously described (Uchime, O., et al. J Biol Chem 291, 89-102, (2016)). Unlabeled and ¹⁵N-labeled protein samples were prepared in 50 mM potassium phosphate, 50 mM NaCl solution at pH 6.0 in 10% D20. All experiments were performed using an independent sample for each experimental measurement as a 400 μL sample in a 5-mm Shigemi; all samples were DMSO matched with 2% d6-DMSO. Correlation ¹H-¹⁵N-HSQC spectra were recorded on ¹⁵N-labeled BAX at 50 μM in the presence and absence of 100 μM of EO. NMR spectra were acquired at 25° C. on a Bruker 600 MHz spectrometer equipped with a cryoprobe, processed using TopSpin and analyzed using NMRView. BAX cross-peak assignments were applied as previously reported (Gavathiotis, E., et al. Nature 455, 1076-1081 (2008)). The weighted average chemical shift perturbation (CSP) was calculated as √(L1δ¹H)²+(L1δ¹⁵N/5)²)/2 in p.p.m. The absence of a bar indicates no chemical shift difference, the presence of a proline, or a residue that is overlapped or missing and therefore not used in the analysis. The significance threshold for backbone amide chemical shift changes was calculated based on the average chemical shift across all residues plus 0.5 or 1 s.d. Solvent-accessible surface area was probed by the addition of 10 mM hy-TEMPO (sigma) to 50 μM ¹⁵N-labeled BAX with and without 100 μM EO measured using standard ¹H-¹⁵N-HSQC with an increased recycle delay of 2 sec. PRE was calculated as the ratio of peak intensities of BAX in the presence of hy-TEMPO to BAX without hy-TEMPO (% intensity). Mapping of chemical shifts and PRE data onto the BAX structure was performed with PyMOL (Schrodinger, LLC, 2018-2019). Software was made available through the SBGrid collaborative network.

### NMR-Based Docking Calculations and Molecular Dynamics

NMR-guided docking of EO into the NMR structure of BAX (PDB: 1F16) was performed using the induced-fit docking (IFD, Schrodinger, LLC, 2018) with extra precision (XP) and a binding site at the midpoint of residues K21, R134, and R145. EO was converted to 3D all-atom structure using LIGPREP (Schrodinger, LLC, 2018) and assigned partial charges with EPIK (Schrodinger, LLC, 2018). Poses generated were consistent with NMR data and indicated a strong favoring of ionic interaction between the carboxylate of EO and a basic residue of BAX. Mutagenesis was used to elucidate the true pose of EO on the trigger site of BAX. The pose consistent with mutant BAX liposomal release data was most consistent with NMR CSP data. This pose was subjected to 3 independent 100 nsec molecular dynamics (MD) simulations using DESMOND (DESMOND, Version 3, Schrodinger, LLC, 2017). Three independent 100 ns MD simulations were also performed with the lowest energy BAX structure from the NMR ensemble (PDB 1F16). MD runs were performed in a truncated octahedron SPC water box using OPLS_2005 force field, 300K, and constant pressure of 1.0325 bar. Analysis of the trajectory was performed with MAESTRO simulation event analysis tools (Schrodinger, LLC, 2018). PyMOL (Schrodinger, LLC, 2018-2019) was used for preparing the highlighted poses. The % ΔRMSF for each residue was calculated as % ΔRMSF=((RMSFEO−RMSFApo)100/RMSFApo), where RMSFEO was the RMSF of an individual MD simulation of EO docked into BAX and RMSFApo is the average RMSF of the apo BAX simulation. Distance frequency histograms were prepared using GraphPad Prism frequency distribution analysis.

### Structural Analysis

Structural analysis was performed in PyMOL (Schrodinger, LLC: NY, 2018-2019) and Maestro tools (Schrodinger, LLC, NY, 2018-2019).

### Cytochrome c Release Assay

BAX^{-/-} or BAK^{-/-} mouse embryonic fibroblasts were maintained in DMEM (Life Technologies) supplemented with 10% FBS, 100 U/mL penicillin/streptomycin, 2 mM 1-glutamine, and 0.1 mM MEM nonessential amino acids. MEFs (5×10⁴ cells/well) were seeded in a 96 well clear u-bottom plate for 18-24 hours. Media was removed and replaced with media lacking FBS, and cells were treated with varying doses of EO for 2 hours at 37° C. After incubation, the media was removed and replaced with 100 μL reaction buffer modified from MEB buffer (150 mM mannitol, 10 mM HEPES-KOH pH 7.5, 50 mM KCl, 0.02 mM EGTA, 0.02 mM EDTA, 0.1% BSA, 5 mM succinate, 20 μg/mL oligomycin, 10 mM DTT, and 0.00125% digitonin) with and without 5 μM BIM-BH3 peptide and incubated at 30° C. for 45 min. After incubation, an additional 100 μL of reaction buffer was added, and the plate was gently tapped to mix. Cytochrome c release was determined by decanting 50 μL of the supernatant and analyzing with the rat/mouse cytochrome c quantikine ELISA kit (R&D Systems, MCTO) according to the recommended protocol. Percentage inhibition was normalized to BIM-BH3 peptide alone (0%) and untreated cells (100%).

### Cell Viability and Caspase-3/7 Activation Assays

3T3 cells were maintained in media identical to that of MEFs. 3T3 cells were seeded (1×10⁴ cells/well) in 96-well opaque plates for 18-24 hours. The media was removed and replaced with media lacking FBS, and cells were treated with EO as a 10× stock in H₂O at the indicated doses for 2 hours before addition of 10% FBS. Cells were then treated with 1 μM each of ABT-263 and S63845. Caspase 3/7 activation was measured at 4 hours by addition of the Caspase-Glo 3/7 chemiluminescence reagent in accordance with the manufacturer's protocol (Promega). Luminescence was detected by an F200 PRO microplate reader (TECAN). Percentage caspase activation was normalized to ABT-263+S63845 alone (100%) and untreated cells (0%). Viability assays were performed at 24 hours by addition of CellTiter-Glo according to the manufacturer's protocol (Promega). Luminescence was detected by an F200 PRO microplate reader (TECAN). Percentage viability was normalized to untreated cells (100%).

### Cellular Thermal Shift Assays (CETSA)

BAK KO MEFs were seeded in a 10 cm dish for 18-24 hours or until approximately 80% confluent. The media was removed and replaced with media lacking FBS, and cells were treated with 10 μM EO as a 10× stock in H₂O or vehicle for 2 hours. The media was then removed, and cells were harvested using a cell scraper and washed twice with PBS. Cells were then resuspended in PBS to 6×10⁶ cells/mL, and 50 μL was transferred to PCR tubes. Cells were then heated in a Biorad C1000 Touch Thermal Cycler for 3 minutes using a temperature gradient (50, 52.1, 55.4, 59.4, 64.9, 69.2, 72.1, and 74° C.). Cells remaining at room temperature (25° C.) served as a control. All cells were lysed by three cycles of freeze-thawing using liquid nitrogen. Samples were then centrifuged at 2×10⁴ g for 15 minutes. The supernatants were collected and resolved by SDS-PAGE with an N-terminal BAX antibody (Cell Signaling, 2772S). Samples were analyzed and quantified using a Li-Cor Odyssey Clx and normalized to 25° C. (100%) and 74° C. (0%).

### Calculation of Recombinant BAX TM

Purified recombinant BAX (25 μM) was combined with DMSO or EO (BAX:EO of 1:10) and loaded into Tycho NT.6 (NanoTemper Technologies) capillaries. First derivative (330/350 nm) melting point curves were generated automatically using the Tycho NT.6 (NanoTemper Technologies). Data was exported and to GraphPad PRISM software for analysis and visualization.

### Statistical Analysis

Statistical significance for pair-wise comparison of groups was determined by 2-tailed Student's t-test using GraphPad PRISM software (Graph Pad Inc., CA). P values of less than 0.05 were considered significant.

### Chemical Syntheses

All chemical reagents and solvents were obtained from commercial sources and used without further purification. Chromatography was performed on a Teledyne ISCO CombiFlash R_{f}200i using disposable silica cartridges. Analytical thin-layer chromatography (TLC) was performed on Merck silica gel plates, and compounds were visualized using ultraviolet (UV). NMR spectra were recorded on a Bruker 600 spectrometer. The Bruker 600 NMR instrument was purchased using funds from NIH award 1S10OD016305. ¹H chemical shifts (δ) are reported relative to tetramethylsilane (TMS, 0.00 ppm) as an internal standard or relative to residual solvent signals. Mass spectra were recorded by the Proteomics Facility at the Albert Einstein College of Medicine.

### Synthesis of EO-1, EO-2

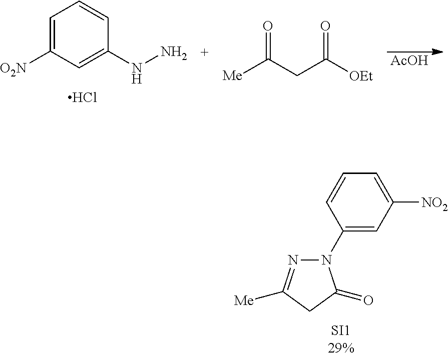

5-methyl-2-(3-nitrophenyl)-2,4-dihydro-3H-pyrazol-3-one (SI1) was synthesized according to the published procedure: 1 Acetic acid (50 mL), ethyl 3-oxobutanoate (1.60 mL, 12.7 mmol, 1.0 equiv.), and (3-nitrophenyl)hydrazine hydrochloride (2.40 g, 12.7 mmol, 1.0 equiv.) were combined in a flask and then heated to 100° C. for 14 hours. Upon standing at room temperature (>60 min), solids formed. These were collected by filtration (filter paper) and washed with water (2×30 mL). After air-drying, S1 was obtained (791 mg, 3.61 mmol, 29%). ¹H NMR (CDCl₃, 300 MHz) δ 8.76 (t, J=2.1 Hz, 1H), 8.35 (ddd, J=8.3, 2.2, 1.0 Hz, 1H), 8.02 (ddd, J=8.2, 2.2, 1.0 Hz, 1H), 7.55 (t, J=8.3 Hz, 1H), 3.50 (d, J=0.7 Hz, 2H), 2.24 (s, 3H).

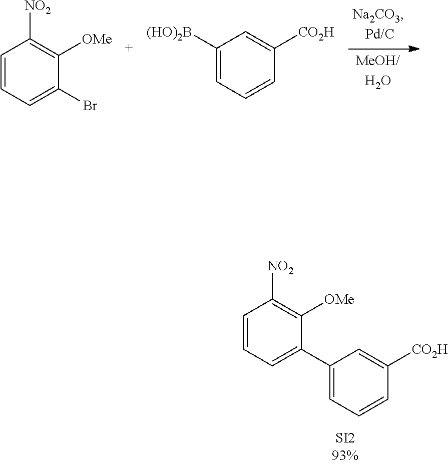 [SMILES: COc1c(Br)cccc1[N+](=O)[O-] ; O=C(O)c1cccc(B(O)O)c1 ; COc1c(-c2cccc(C(=O)O)c2)cccc1[N+](=O)[O-]]

2′-methoxy-3′-nitro-[1,1′-biphenyl]-3-carboxylic acid (SI2) was prepared as previously described: 2 To a round bottom flask was added 1-bromo-2-methoxy-3-nitrobenzene (3.0 g, 13 mmol, 1.0 equiv.), 3-boronobenzoic acid (2.6 g, 16 mmol, 1.2 equiv.), sodium carbonate (2.7 g, 26 mmol, 2.0 equiv.), water (30 mL) and methanol (30 mL). Argon was bubbled through the solution for 10 min. and Pd/C (Degussa, 5% Pd, 2.2 g, 1.0 mmol, 8 mol %) was added. The flask was then sealed with a septum/Ar balloon, and heated to 60° C. After two hours, TLC analysis (Hexanes:EtOAc 1:1; UV) showed full conversion, and the mixture was cooled to room temperature. The catalyst was removed by filtration through celite, and the product was then precipitated by the addition of 1 M HCl (50 mL). The solids were collected by filtration, washed with water, and air-dried to give the product as an off-white solid (3.3 g, 12 mmol, 93%).

TLC: Rf=0.27 (Hexanes:EtOAc 1:1; UV). ¹H NMR (DMSO-d6, 600 MHz) δ 13.2 (bs, 1H), 8.11 (s, 1H), 8.02 (d, J=7.7 Hz, 1H), 7.92 (dd, J=8.1, 1.1 Hz, 1H), 7.83, (d, J=7.6 Hz, 1H), 7.74 (dd, J=7.7, 1.1 Hz, 1H), 7.64 (t, J=7.7 Hz, 1H), 7.44 (t, J=7.9 Hz, 1H), 3.44 (s, 3H).

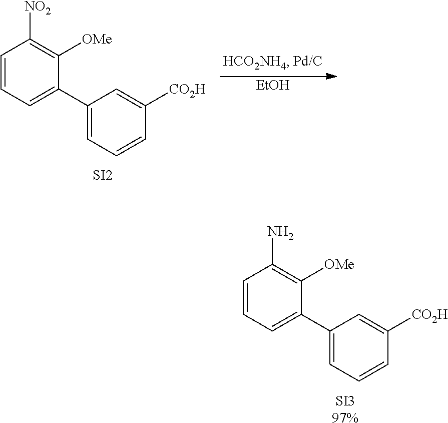

3′-amino-2′-methoxy-[1,1′-biphenyl]-3-carboxylic acid (SI3) was synthesized according to the published procedure: 3 A round bottom flask was purged with argon and 2′-methoxy-3′-nitro-[1,1′-biphenyl]-3-carboxylic acid (1.50 g, 5.49 mmol, 1.0 equiv.), ammonium formate (3.30 g, 52.3 mmol, 9.5 equiv.), Pd/C (Degussa, 5% Pd, 1.50 g, 0.71 mmol, 13 mol %), and ethanol (180 mL). The flask was sealed with a septum/empty balloon. The reaction mixture was then heated to 80° C. for 10 min. and MS analysis showed complete conversion. The mixture was cooled to room temperature and filtered through celite. Removal of the volatiles gave the aniline SI-3 (1.29 g, 5.30 mmol, 97%) in good purity. The product was used for the next step without further purification. ¹H NMR (DMSO-d6, 600 MHz) δ 8.06 (t, J=1.6 Hz, 1H), 7.88 (dt, J=7.7, 1.3 Hz, 1H), 7.65, (dt, J=7.7, 3.3 Hz, 1H), 7.47 (t, J=7.7 Hz, 1H), 6.89 (t, J=7.7 Hz, 1H), 6.72 (dd, J=7.9, 1.5 Hz, 1H), 6.51 (dd, J=7.6, 1.6 Hz, 1H), 5.02 (bs, 2H), 3.28 (s, 3H). ¹³C NMR (151 MHz, DMSO-d6) δ 168.4, 143.9, 142.3, 138.9, 134.5 (broad), 134.1, 132.1, 129.9, 128.4, 128.2, 125.0, 117.9, 115.2, 59.3.

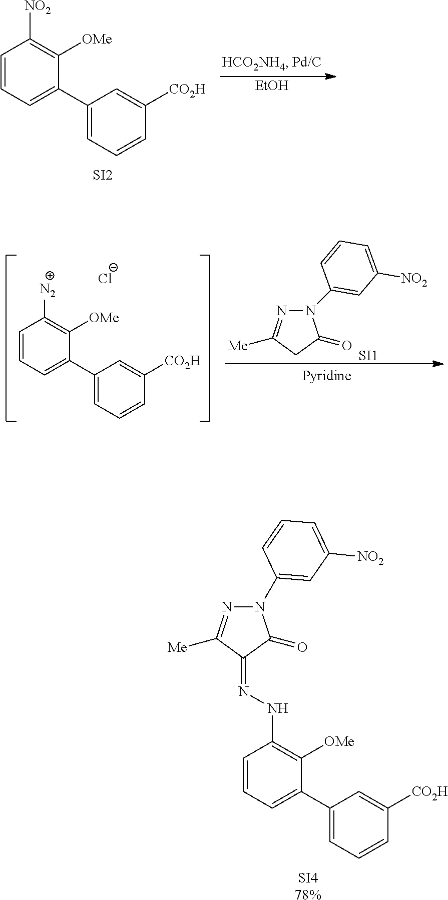

2′-methoxy-3′-(2-(3-methyl-1-(3-nitrophenyl)-5-oxo-1,5-dihydro-4H-pyrazol-4-ylidene)hydrazineyl)-[1,1′-biphenyl]-3-carboxylic acid (SI4; EO-2) Aniline SI3 (333 mg, 1.37 mmol, 1.5 equiv.) and water (5 mL) were added to a flask and cooled in an ice/water bath. HCl (37%, 0.75 mL, 9.12 mmol, 10 equiv.) was quickly added followed by an aqueous suspension of sodium nitrite (107 mg, 1.55 mmol, 1.7 equiv.). The resulting brown/orange mixture was stirred for 5 min. and then added over 1 min. to a cooled (ice/water) solution of SI1 (200 mg, 0.91 mmol, 1.0 equiv.) in pyridine (5 mL). A bright red solid formed immediately. After 10 min the solids were collected by filtration and washed thoroughly with water and then rinsed with EtOAc. The red solids (432 mg, 0.71 mmol, 78%) were used for the next reaction without further purification. ¹H NMR (DMSO-d6, 600 MHz) δ 13.64 (s, 1H), 13.13 (bs, 1H), 8.77 (s, 1H), 8.39 (d, J=8.2 Hz, 1H), 8.17 (s, 1H), 8.08 (d, J=8.3 Hz, 1H), 8.01 (d, J=7.6 Hz, 1H), 7.87 (d, J=7.3 Hz, 1H), 7.81 (d, J=8.3 Hz, 1H), 7.78 (t, J=8.6 Hz, 1H), 7.65 (t, J=7.4 Hz, 1H), 7.39 (t, J=8.1 Hz, 1H), 7.32 (d, J=7.7 Hz, 1H), 3.48 (s, 3H), 2.29 (s, 3H). ESI-MS: calc'd for C₂₄H₂₀N₅O₆ (M+H)+474.1408 found 474.1406.

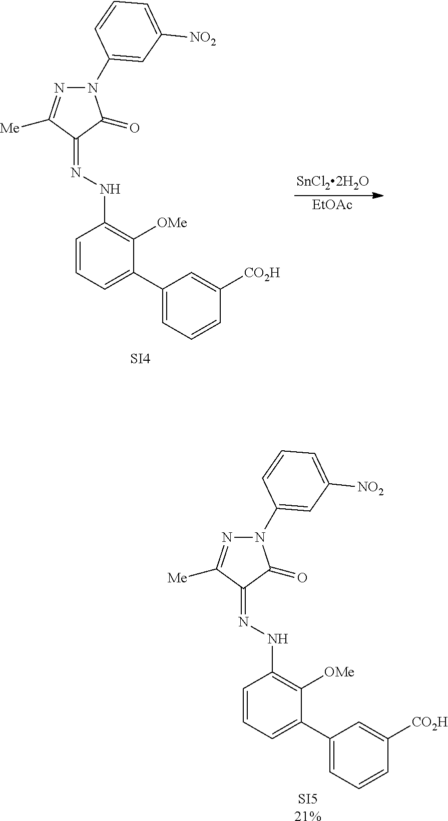

(Z)-3′-(2-(1-(3-aminophenyl)-3-methyl-5-oxo-1,5-dihydro-4H-pyrazol-4-ylidene)hydrazineyl)-2′-methoxy-[1,1′-biphenyl]-3-carboxylic acid (SI5; EO-1): EO-2 (100 mg, 0.21 mmol, 1.0 equiv.), tin(II) chloride dihydrate (143 mg, 0.63 mmol, 3.0 equiv.), and EtOAc (5 mL) were combined in a tube, which was then sealed and placed in a pre-heated oil bath (80° C.). Over time, the solution turned bright red and almost homogeneous. After 15 hours, the reaction mixture was cooled to room temperature, concentrated and purified by reverve phase chromatography (5.5 g C18 g column. 0-60% MeCN in water (each with 0.1% TFA)). This resulted in a sample of pure EO-1 (20 mg, 45 μmol, 21%) as well as a larger amount of contaminated EO-¹H NMR (DMSO-d6, 600 MHz) δ δ 13.79 (s, 1H), 8.17 (t, J=1.8 Hz, 1H), 8.00 (dt, J=7.7, 1.3 Hz, 1H), 7.87 (dt, J=7.7, 1.5 Hz, 1H), 7.79 (dd, J=8.1, 1.6 Hz, 1H), 7.65 (t, J=7.7 Hz, 1H), 7.38 (t, J=7.9 Hz, 1H), 7.29 (dd, J=7.7, 1.6 Hz, 1H), 7.21-7.17 (m, 1H), 7.14-7.09 (m, 1H), 7.07 (t, J=7.9 Hz, 1H), 6.46-6.39 (m, 1H), 3.46 (s, 3H), 2.33 (s, 3H). ¹³C NMR (151 MHz, DMSO-d6) δ 167.10, 157.16, 147.92, 145.68, 138.52, 136.92, 134.59, 133.84, 132.94, 131.22, 129.47, 129.36, 129.10, 128.71, 127.20, 125.78, 114.35, 60.95, 11.58. ESI-MS: calc'd for C₂₄H₂₂N₅O₄.

### Example 2

### Eltrombopag Binds to BAX

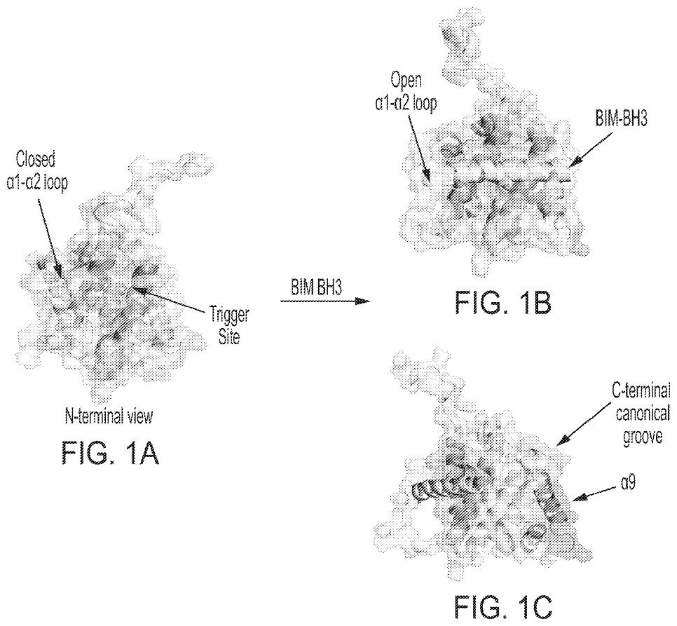

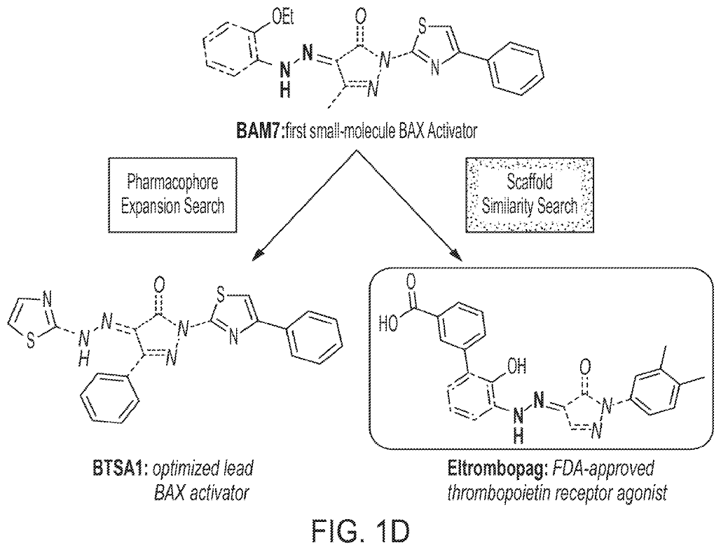

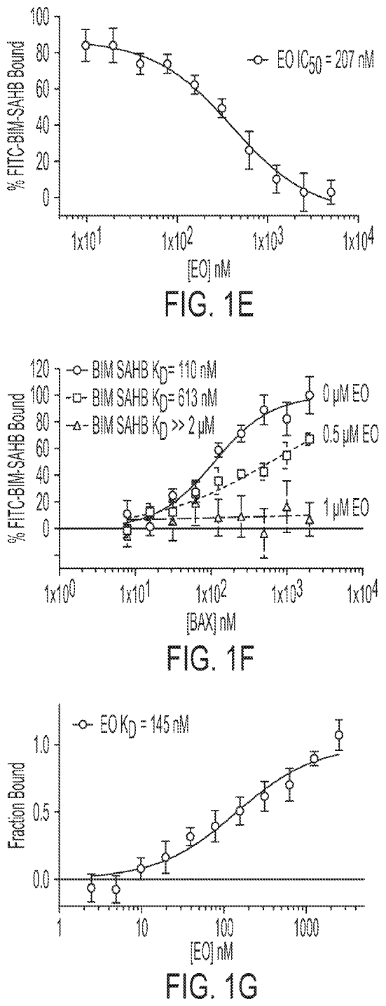

To identify small-molecule BAX modulators, pharmacophore enhancement and substructure similarity computational methods based on the small molecule activator of BAX, BAM7, were explored (Gavathiotis, E., et al. Nat Chem Biol 8, 639-645 (2012)). Specifically, upon searching a library of FDA-approved small molecules using the 3-methyl pyrazolone and phenylhydrazone core as a query, eltrombopag (EO) was identified as a hit (FIG. 1d). EO bears a striking similarity to BAM7, sharing the 3-methyl pyrazolone and phenylhydrazone core. However, substitutions at either side to this core with dimethylphenyl and benzoic acid markedly distinguish EO from BAM7 and the optimized lead BAX activator, BTSA1 (FIG. 1d) ((Gavathiotis, E., et al. Nat Chem Biol 8, 639-645 (2012); Reyna, D. E., et al. Cancer Cell 32 490-505 (2017)).

It was hypothesized EO would exhibit some binding interaction with the N-terminal BAX trigger site. A competitive fluorescence polarization assay (FPA) based on the interaction between recombinant BAX and fluorescently-labeled stapled BIM-BH3 peptide (FITC-BIM-SAHB) was therefore used (Gavathiotis, E., et al. Nature 455, 1076-1081 (2008)). EO dose-dependently competed against FITC-BIM-SAHB with a remarkable half-maximal inhibition IC50 of 207 nM (FIG. 1e). Titrations of BAX to a constant concentration FITC-BIM-SAHB exhibited a decreased affinity in the presence of constant concentrations of EO (FIG. 1f). Direct binding of EO to BAX was demonstrated using microscale thermophoresis (MST), as previously established for BAX, with a calculated dissociation constant KD of 143 nM (FIG. 1g) (Amgalan D, et al. Nature Cancer volume 1, pages 315-328(2020)). The IC50 and KD are consistent with a competitive binding mechanism where EO directly displaces FITC-BIM-SAHB from the N-terminal trigger site of BAX.

### Eltrombopag Inhibits BAX Activation

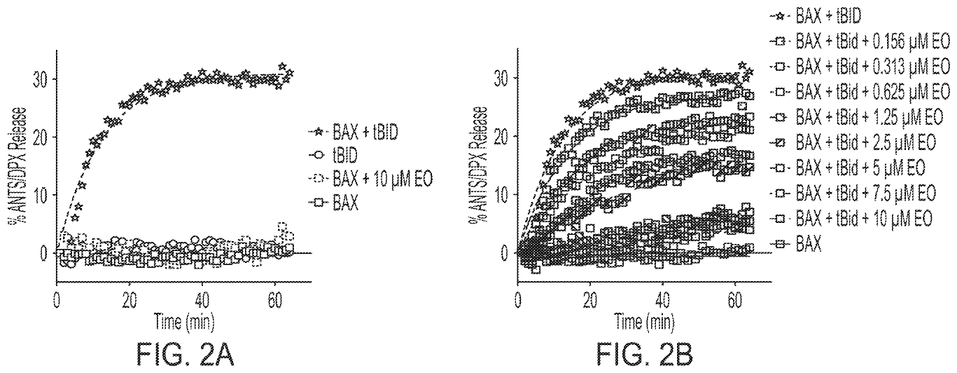

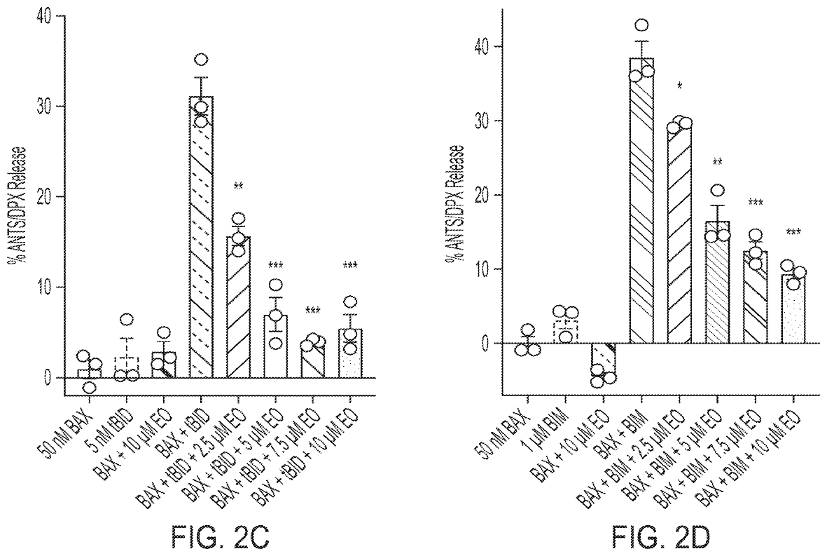

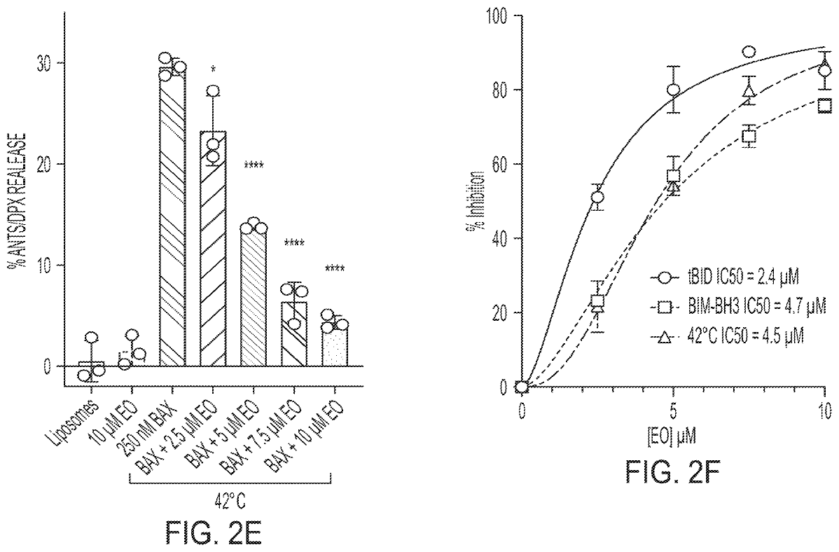

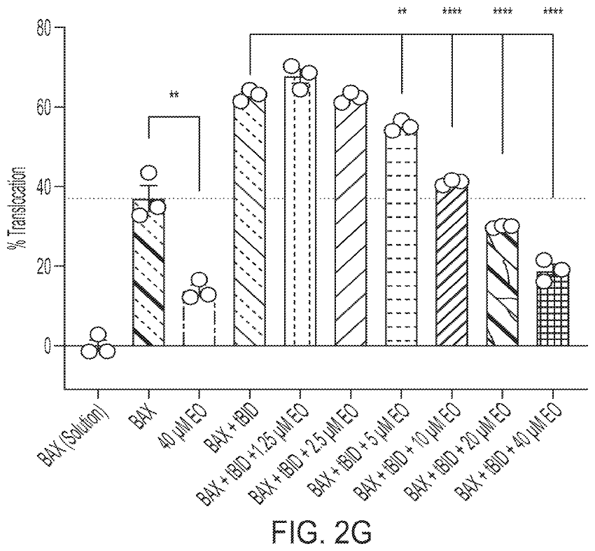

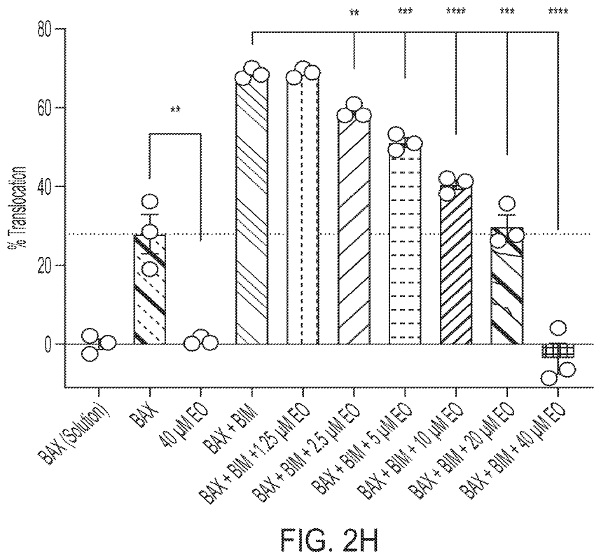

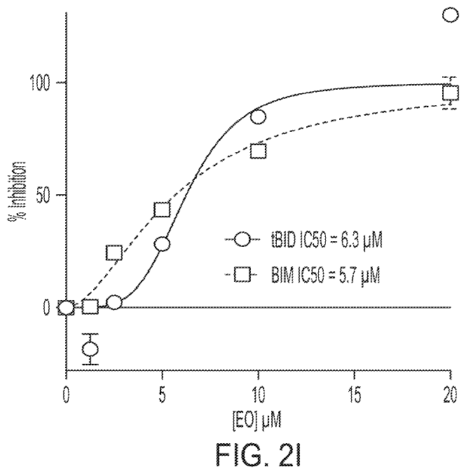

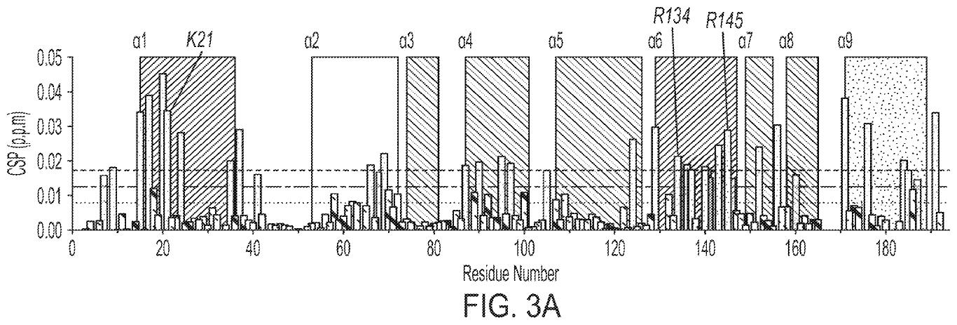

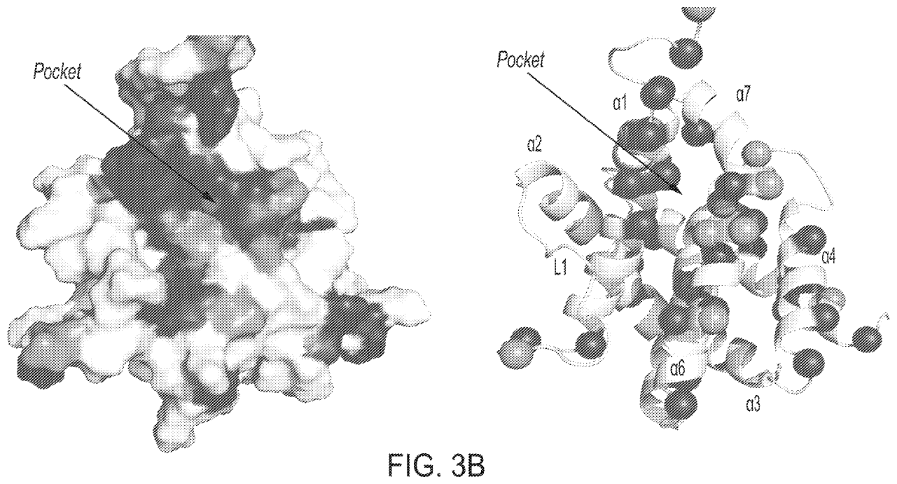

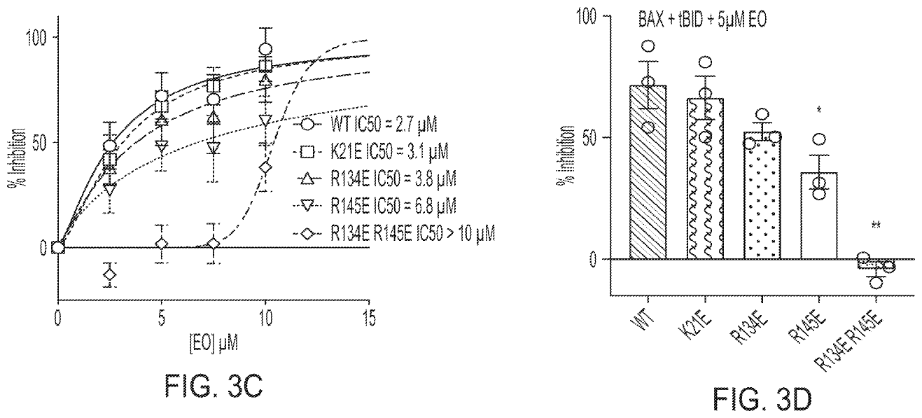

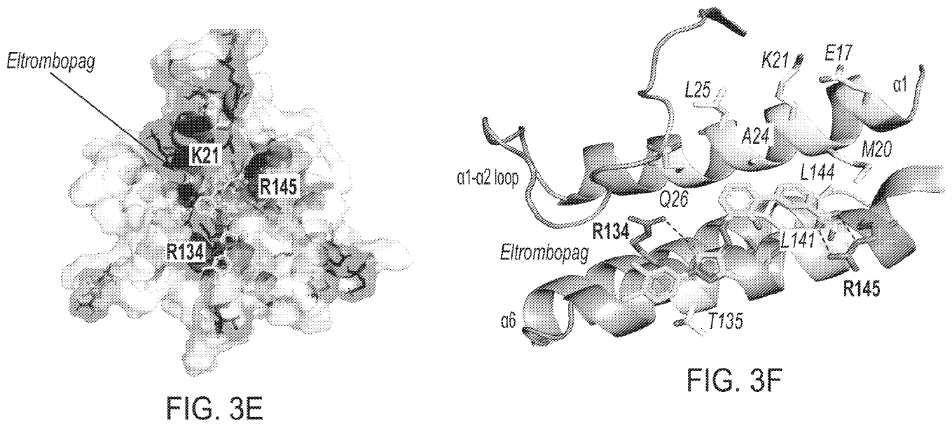

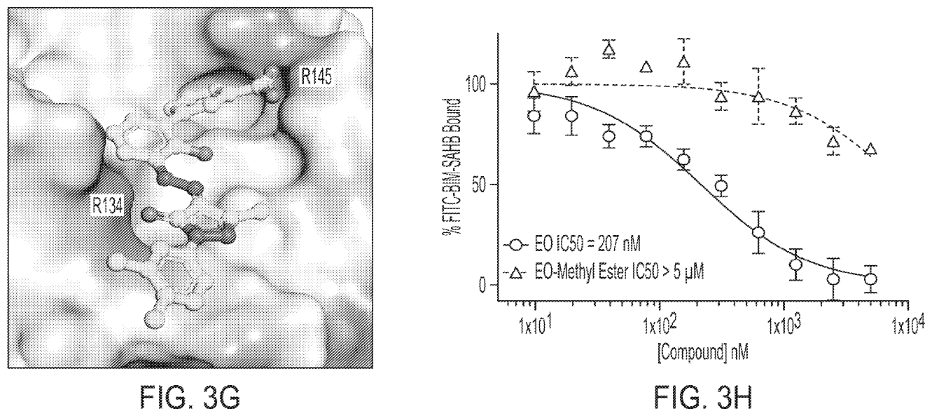

Next, EO's capacity to modulate BAX activity was evaluated using liposomal release assays. EO exhibited no ability to activate recombinant BAX in concentrations even up to 10 μM (FIG. 2a). Instead, EO was able to inhibit both tBID- and BIM BH3-mediated BAX activation (FIGS. 2b, 3c, and 2d). Based on the competitive nature of EO binding to the N-terminal BAX trigger site, it was predicted that EO inhibition of BAX activity would be dependent on the BH3-only activator concentration. Indeed, inhibition of tBID-mediated BAX activation by EO was inversely proportional to the concentration of the tBID activator, consistent with competitive inhibition of BH3-activator binding. Furthermore, EO was capable of inhibiting heat-induced BAX activation, indicating that EO can stabilize inactive BAX in addition to blocking of the BAX activation site from BH3-mediated activators (FIG. 2e). EO exhibited similar low micromolar potency in inhibiting BAX activity in liposomal release assay against all stimuli (tBID IC50=2.4 μM, BIM IC50=4.7 μM, heat IC50=4.5 μM). (FIG. 2f).

Prior to permeabilizing membranes, activated BAX must first translocate to the membrane. In order to explore this earlier step in BAX activation, an NBD-fluorescence based translocation assay was used (FIGS. 2g, 2h, and 2i). EO was capable of inhibiting tBID- and BIM BH3-induced BAX translocation dose-responsively with comparable potency to that of inhibiting liposomal release (tBID IC50=6.3 μM, BIM IC50=5.7 μM). Similarly, EO was capable of inhibiting heat-induced auto-translocation of BAX, indicating that EO binding stabilizes an inactive conformation of BAX. One of the earliest conformational changes of BH3-mediated BAX activation is the exposure of an N-terminal epitope, requiring opening of the α1-α2 loop from its inactive conformation, which is recognized by an anti-6A7 epitope-specific antibody (Suzuki, M., et al. Cell 103, 645-654 (2000); Gavathiotis, E., et al. Mol. Cell 40, 481-492 (2010)). In an immunoprecipitation assay using the anti-6A7 epitope-specific antibody, BIM-BH3 induced 6A7 exposure as previously shown, but it was inhibited by the presence of EO. Taken together, the data indicate EO inhibits BAX activation process at an early stage by blocking activator binding and stabilizing a soluble inactive form of BAX.

### Eltrombopag Forms Unique Contacts at the BAX Trigger Site

To determine the binding site of EO, 2D ¹H-¹⁵N heteronuclear single quantum coherence (HSQC) NMR analysis with ¹⁵N-labeled BAX was performed. EO titration shifted select cross-peaks of corresponding BAX residues in the NMR spectra. Analysis of the chemical shift perturbations (CSPs) of BAX in the presence of EO indicated small and specific shifts, as with previous NMR studies of BAX (Gavathiotis, E., et al. Nature 455, 1076-1081 (2008); Gavathiotis, E., et al. Mol. Cell 40, 481-492 (2010); Gavathiotis, E., et al. Nat Chem Biol 8, 639-645 (2012); Reyna, D. E., et al. Cancer Cell 32 490-505 (2017)). Significant CSPs were localized predominantly to the N-terminal BAX trigger site, specifically the N-terminal region of α1 and the length of α6 (FIG. 3a). Mapping of the CSPs onto the inactive BAX structure revealed that CSPs localizing to the BAX trigger site coalesce to form a contiguous surface with a shallow hydrophobic pocket between α1 and α6. (FIG. 3b). Additional CSPs corresponding to residues in adjacent helices to the trigger site, α4, and α7 but also distant at the C-terminal α9 were observed (FIGS. 3a and 3b). Previous crystal structures of the inactive BAX mutants P168G and W139A suggested that binding at the N-terminal trigger site may modulate BAX activity via local conformational changes at α4, α7, and α9 (Dengler, M. A., et al. Cell Rep. 27 359-373 (2019)). NMR analysis of BAX activation with BIM SAHB and small-molecule trigger site activators also highlighted allosteric sensing in α4, α7, and α9 (Gavathiotis, E., et al. Nature 455, 1076-1081 (2008)). It is possible that these distal CSPs correspond to similar allosteric conformational effects from binding of EO to the BAX trigger site. Notably, few chemical shift perturbations in N-terminal α1-α2 loop residues were observed, indicating that its structure remains largely unchanged upon EO binding (FIGS. 3a and 3b). This is in direct contrast with BIM-SAHB and BTSA1 binding, which induce significant CSPs in α1-α2 loop residues and corresponding with a displacement of the α1-α2 loop from the trigger site, a critical step in the activation of BAX.

Next, molecular docking of EO to the BAX surface guided by the CSPs was performed to determine the binding pose of EO to the inactive BAX structure (PDB: 1F16) (Suzuki, M., et al. Cell 103, 645-654 (2000)). Ligand preparation for docking of EO using Schrodinger LigPrep at pH 7±1 expectedly yielded EO exclusively with a deprotonated anionic carboxylate group. It was predicted that this negative charge would form a favorable interaction with one of the three basic residues at the EO binding interface highlighted by the NMR data, K21, R134, or R145, all of which exhibited significant CSPs (FIG. 3a). Molecular docking of EO was therefore performed to a site centered about residues K21, R134, and R145. Docking was performed using induced-fit docking (IFD) approach with Schrodinger tools with a largely extended surface of BAX at the trigger site to account for potential ambiguity with the NMR data and exhaustively consider possible binding modes and the associated local conformational changes of α1, α6, and α1-α2 loop residues. The IFD yielded several poses which featured ionic interactions between the EO-carboxylate and K21, R134, or R145 as expected.

It was then determined which EO pose was appropriate by comparing BAX trigger site mutants that would eliminate one of the three basic trigger site residues, K21E, R134E, or R145E, to wild-type (WT). All tested mutants exhibited identical retention time in size-exclusion chromatography analysis suggesting no effect on BAX folding and had very similar purity and molecular weight as WT BAX as determined by SDS-PAGE. In liposomal release assays, all of the BAX mutants were functional, although R134E and R145E exhibited lower ANTS/DPX release in response to tBID activation. Of the mutants tested, only BAX R145E exhibited a reduced inhibition in response to EO, with an IC50 more than double that of BAX WT (FIGS. 3c and 3d). Notably, BAX K21E exhibited reduced activation in response to BIM BH3, BAM7, and BTSA1 activators but not reduced inhibition in response to EO (Gavathiotis, E., et al. Nature 455, 1076-1081 (2008); Gavathiotis, E., et al. Cell 40, 481-492 (2010)). This highlights how unique contacts at the trigger site could determine whether compounds will behave as BAX activators or inhibitors.

The loss of EO-mediated BAX inhibition with the R145E mutant indicates that EO forms a critical interaction via the anionic carboxylate with BAX R145. With this known, the EO docking poses were reevaluated, and the top pose featuring an ionic interaction between the EO-carboxylate and sidechain of R145 was analyzed (FIGS. 3e, 3f, and 3g). In addition to the ionic interaction, this pose also features hydrophobic interactions between the biphenyl moiety of EO and the hydrophobic pocket formed by residues L24, M137, G138, and L141 between α1 and α6. Furthermore, the docking pose features contacts at the N-terminal of α6 unique to poses possessing an ionic interaction with R145. Of particular note is a hydrogen bond between R134 side chain and the carbonyl of the pyrazolone core of EO, which could potentially explain the slight trend towards weaker inhibition of BAX R134E (FIGS. 3c and 3d). It was therefore hypothesized that the double mutant BAX R134E R145E would markedly reduce EO inhibition of BAX. Indeed, EO exhibited clearly weak inhibition of BAX R134E R145E with an IC50>10 μM (FIGS. 3c and 3d). Consistently, in the presence of EO, this double mutant also exhibited minimal CSPs of ¹⁵N-labeled BAX by HSQC-NMR studies and markedly reduced affinity as determined by MST.

To further probe the specificity of EO for the BAX trigger site and the critical R145 ionic interaction, an EO analog featuring a methyl ester (EO-Methyl Ester) in place of the carboxylic acid was utilized. The addition of a methyl group eliminates the anionic carboxylate as well as adds steric bulk at the site of the critical R145 interaction (FIGS. 3e, 3f, and 3g). EO-Methyl Ester exhibited minimal competition of FITC-BIM-SAHB by FPA (IC50>5 μM), indicating a dramatically diminished binding affinity (FIG. 3h). Consistently, EO-Methyl Ester induced minimal CSPs of ¹⁵N-labeled BAX by HSQC-NMR studies and exhibited significantly diminished inhibition of BAX in liposomal release assays. Taken together, the data show that EO binds to the N-terminal trigger site of BAX, forming contacts with a shallow hydrophobic pocket between α1 and α6, interacting predominantly with α6 residues. Notably, this pocket is formed adjacent to the α1-α2 loop, which interacts with residues of α1 and α6 and it is not disturbed by EO binding. Docking, mutagenesis, and the EO-Methyl Ester indicate that EO makes a critical contact with R145 via the anionic carboxylate as well as a secondary hydrogen bond with R134 via the pyrazolone carbonyl.

### Stabilization of Inactive BAX by Eltrombopag

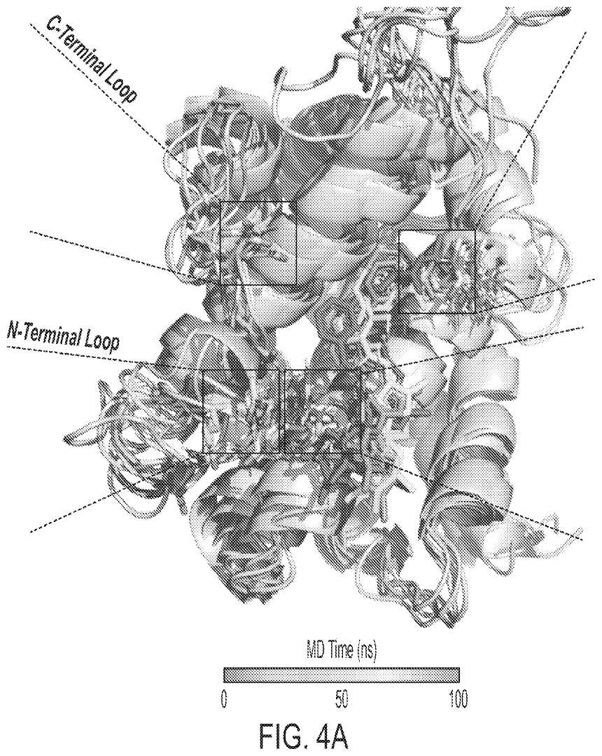

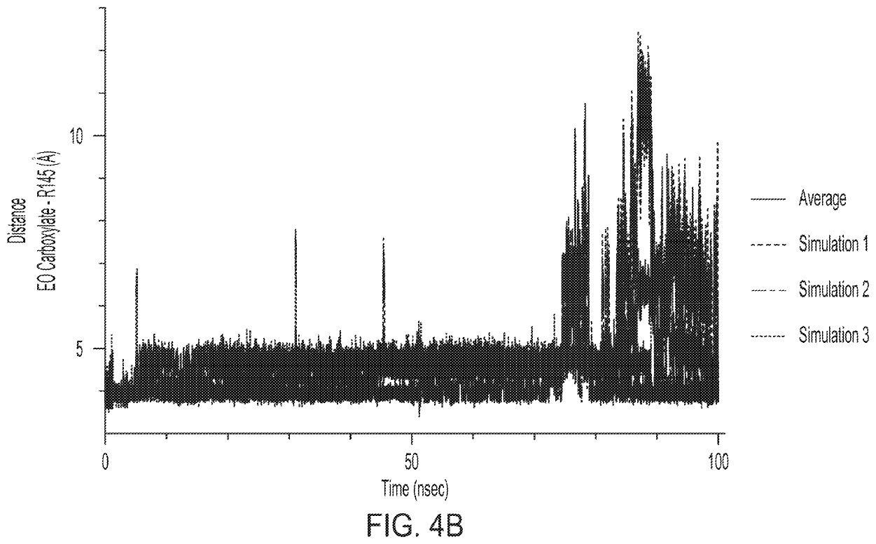

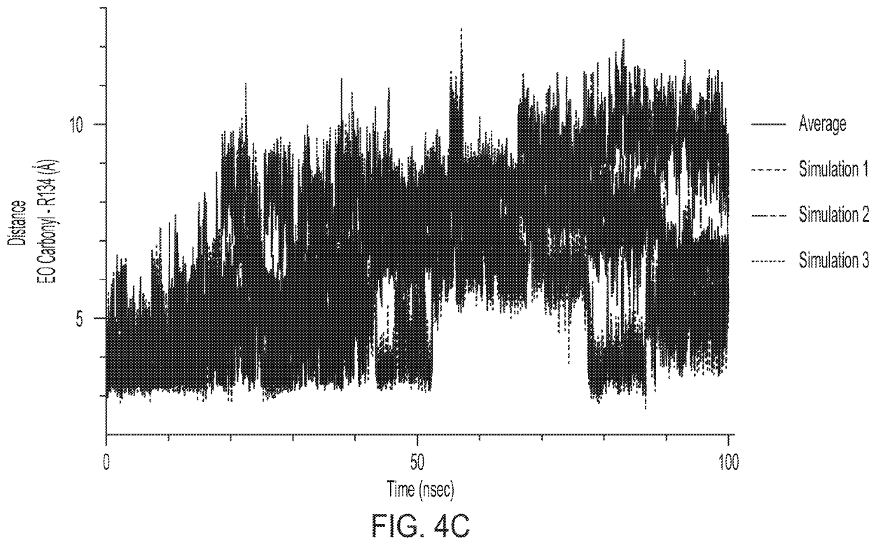

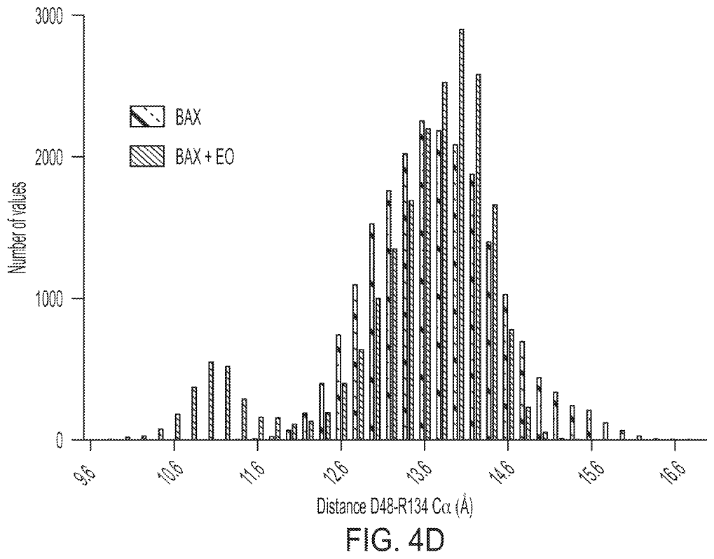

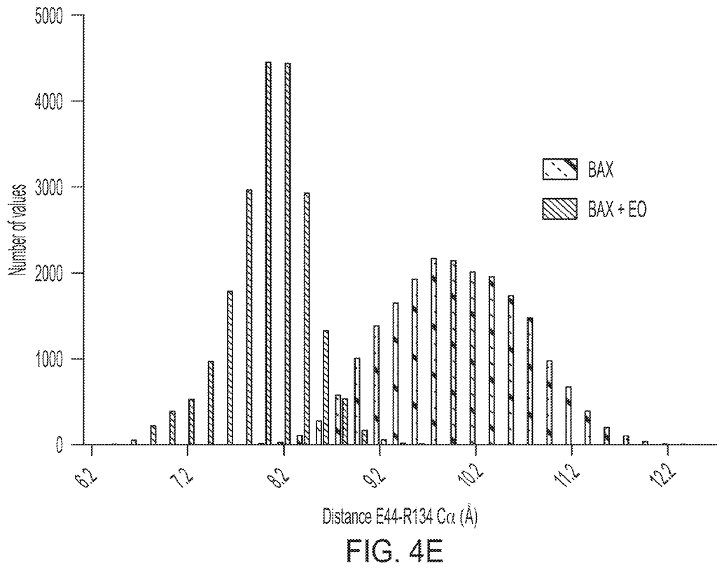

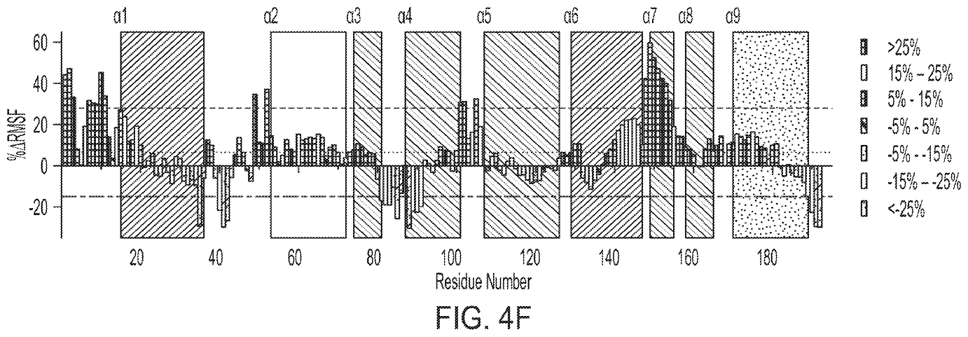

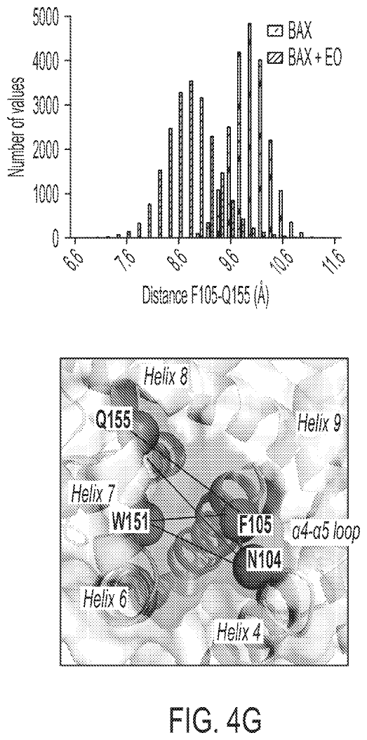

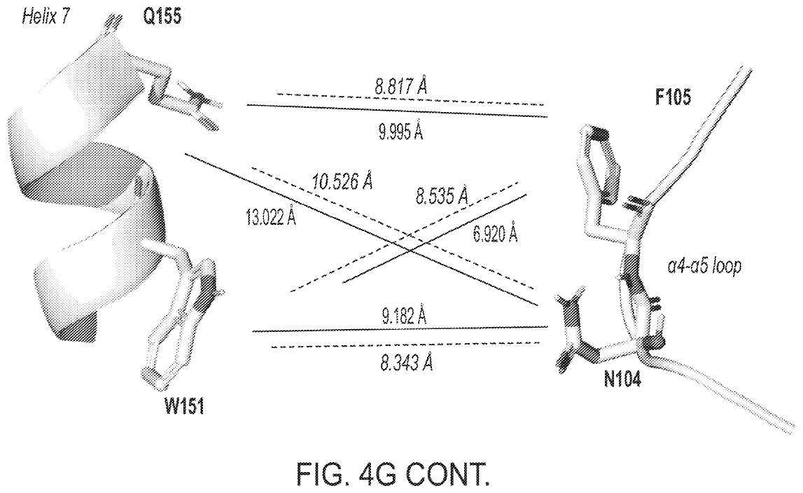

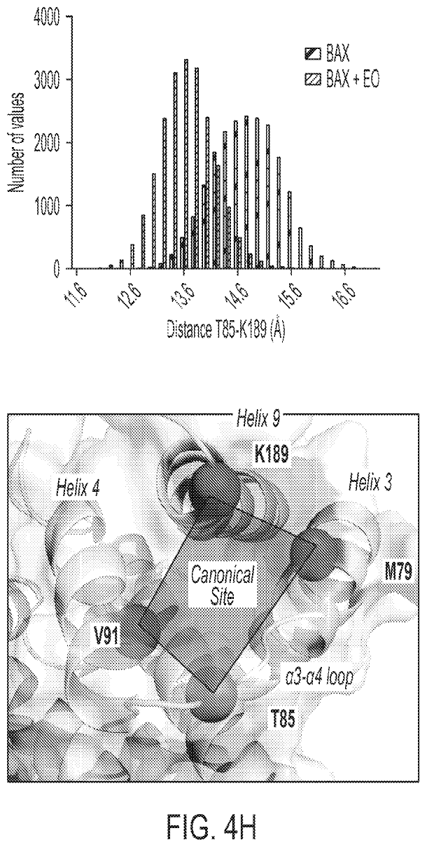

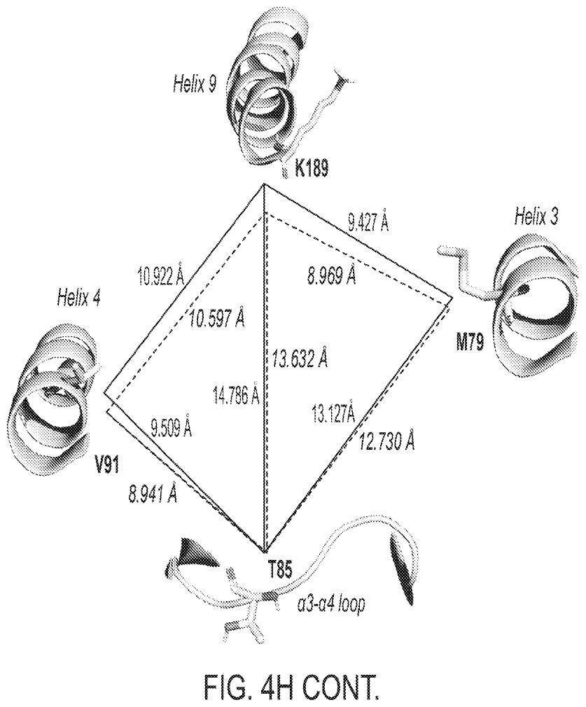

The data indicate that the N-terminal BAX trigger site, which has been established as the binding site for both BH3-only proteins and small molecule activators, can also be an inhibitory site. NMR and mutagenesis data indicated that the distinct contacts and binding mode of EO may be responsible for the inhibitory activity of EO. To explore how EO can accomplish inhibition of BAX conformation and activity, three independent molecular dynamics (MD) simulations of the BAX-EO complex and of the inactive BAX structure were performed. The overall structure of BAX was maintained in all six simulations with an average r.m.s. deviation and radius of gyration of 4.17±0.44 Å and 16.36±0.11 Å for the backbone atoms without EO compared to average r.m.s. deviation and radius of gyration of 4.53±0.76 Å and 16.22±0.08 Å bound to EO. In the three MD simulations of the BAX-EO complex, EO remains in a stable conformation (FIG. 4a). The distance between R145 and the EO-carboxylate remains stable throughout the simulation (FIG. 4b). The interaction between R134 and the EO carbonyl is noticeably more dynamic. However, the two groups remain in close proximity throughout the simulation (FIG. 4c). The EO-BAX distances strongly support the mutagenesis data, and binding mode wherein EO forms a critical interaction at R145 and a secondary weaker interaction at R134.

HSQC CSPs suggested that EO does not cause significant conformational changes to the α1-α2 loop, in contrast with other trigger site binders, BTSA1 and BH3 peptides. In the unbound BAX structure, R134 on α6 sits in close proximity to E44 and D48 on the α1-α2 loop (Suzuki, M., et al. Cell 103, 645-654 (2000)). The distance between R134 and D48 is approximately equal for both the BAX and BAX-EO simulations (FIG. 4d). However, R134 and E44 remain in closer proximity in simulations of the BAX-EO complex than in BAX alone (FIG. 4e). Furthermore, the BAX-EO simulations displayed a narrower distribution of distances indicating reduced conformational flexibility in the N-terminal region of the α1-α2 loop. Notably, the few CSPs observed on the α1-α2 loop were towards the N-terminal region (FIG. 3a).

To further explore potential conformational changes associated with EO binding to BAX the percentage change in root mean square fluctuation (RMSF), a measure of the dynamics of each residue in the BAX structure, was analyzed (FIG. 4f). Residues towards the N and C-terminus of α1-α2 loop exhibit reduced and increased RMSF, respectively, as expected based on the α1-α2 loop distances to R134. Two additional regions exhibited dramatic changes in RMSF. The α4-α5 loop and helix α7 exhibit an increase in RMSF, whereas α3-α4 loop and the C-terminal helix α9 exhibit a decrease in RMSF.

The interface between the α4-α5 loop and α7 has been suggested as a potentially important site for communication between the N-terminal trigger site and the C-terminal canonical site (Gavathiotis, E., et al. Mol. Cell 40, 481-492 (2010); Dengler, M. A., et al. Cell Rep. 27, 359-373 (2019)). Crystal structures of inactive BAX mutants (P168G and W139A) display changes in the interface of the α4-α5 loop and α7, with notable changes in the conformation of F105 and W151. The distances between the α4-α5 loop and α7 were measured, and reduced distances between most of the residues in the presence of EO were observed with the exception of the distance between F105 to W151 which increases (FIG. 4g). Furthermore, changes in the distances between R89 and W139 on α4 and α6, respectively, as well as in the distances between R89 and F93 on α6 were observed, both of which showed changes in the inactive BAX crystal structures (Dengler, M. A., et al. Cell Rep. 27, 359-373 (2019)). The MD data strongly agrees with the crystallographic structures of inactive BAX mutants and point to a potential mechanism by which binding at the trigger site propagates conformational changes throughout BAX.

The α3-α4 loop and α9 form what can be considered as the opening of the canonical site. In order for BAX to translocate to the mitochondria, α9 must partially dissociate from the canonical site of BAX. To evaluate this, the distances between four residues forming the boundaries of the opening to the canonical site were measured (FIG. 4h). All of the distances measured were reduced. By approximating the canonical site opening as two triangles, the approximate canonical site opening area of BAX and the BAX-EO complex was calculated as 113 Å² and 102 Å², respectively, a reduction of approximately 9%. In summary, the MD data indicate that EO binding at the BAX trigger site induces direct and allosteric conformational changes consistent with stabilization of the inactive soluble BAX structure. These include changes in the interfaces of α4 and α6 as well as the α4-α5 loop and α7, which may allosterically couple the trigger site to α9 and the canonical site.

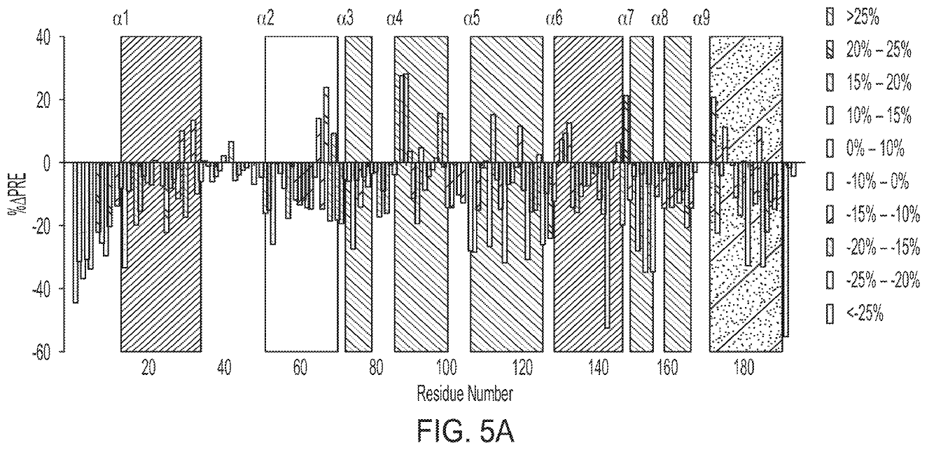

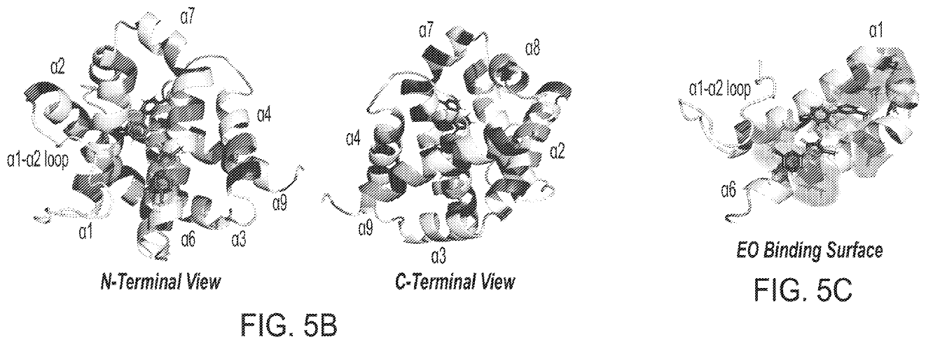

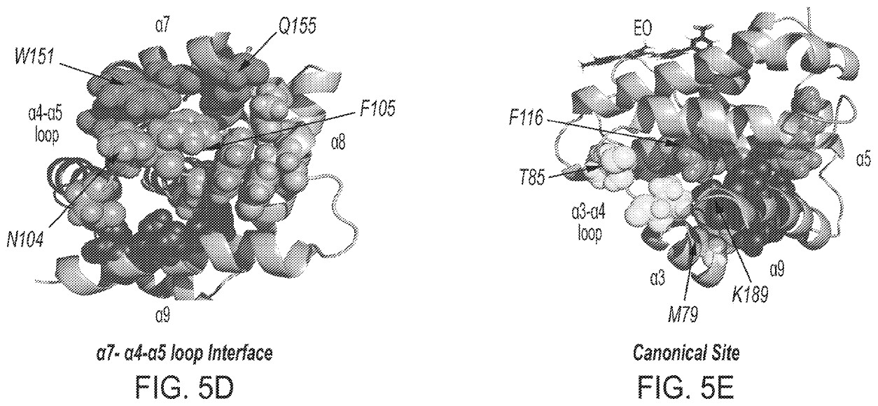

To independently assess the results of MD simulations, paramagnetic relaxation enhancement (PRE) effects on ¹⁵N-labeled BAX caused by a soluble paramagnetic probe, hy-TEMPO, were measured in the presence and absence of EO. The hy-TEMPO probe is a small sparsely functionalized molecule that can bind nonspecifically to solvent-exposed surfaces and pockets on the surface of BAX. It was observed that the presence of EO altered the PRE effects not only by directly blocking hy-TEMPO binding to the trigger site but by allosterically altering the surface topology of BAX (FIG. 5a). Mapping these changes to the surface of BAX revealed that EO binding protected the trigger site residues in direct contact with EO based on the docking pose, particularly around the hydrophobic pocket formed between α1 and α6 (FIGS. 5b and 5c). As expected, PRE effects on the α1-α2 loop were unchanged in the presence of EO, consistent with this loop remaining closely associated with the trigger site. Furthermore, reduction in PRE effects was observed on residues surrounding the interface of α7 and the α4-α5 loop as well as internal residues of the canonical site such as α3, α5, and α9 (FIGS. 5d and 5e). This reduction in PRE effects at the interface of α7 and the α4-α5 loop is consistent with the closer association predicted by MD simulations (FIG. 4). Furthermore, the reduction in PRE effects on internal canonical site residues is consistent with stabilization of α9 binding at the canonical site and a narrowing of the canonical site opening as suggested by MD simulations (FIG. 4). The PRE effects on these regions strongly corroborate the findings from biochemical, NMR, and molecular dynamics data and further support that binding events at the BAX trigger site can induce allosteric conformational changes in the canonical site via changes in α4, α4-α5 loop, and α7.

### Eltrombopag Inhibits BAX-Mediated Apoptosis

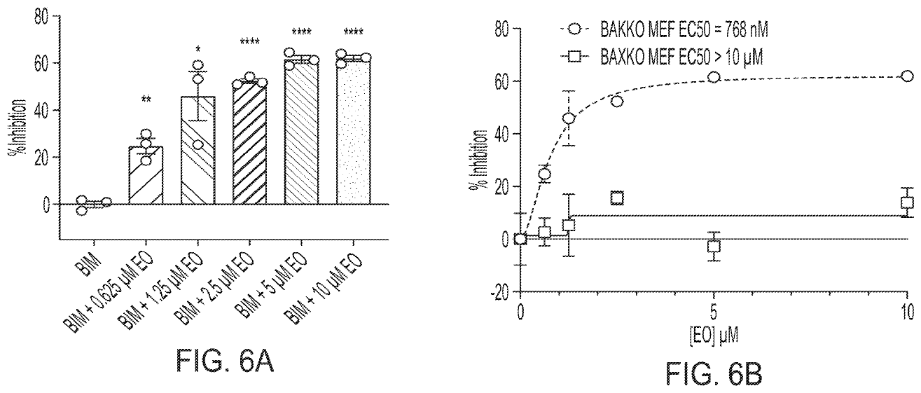

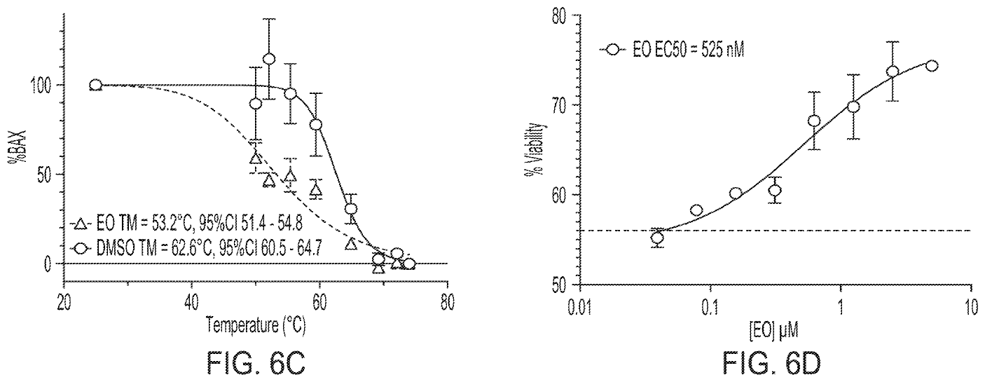

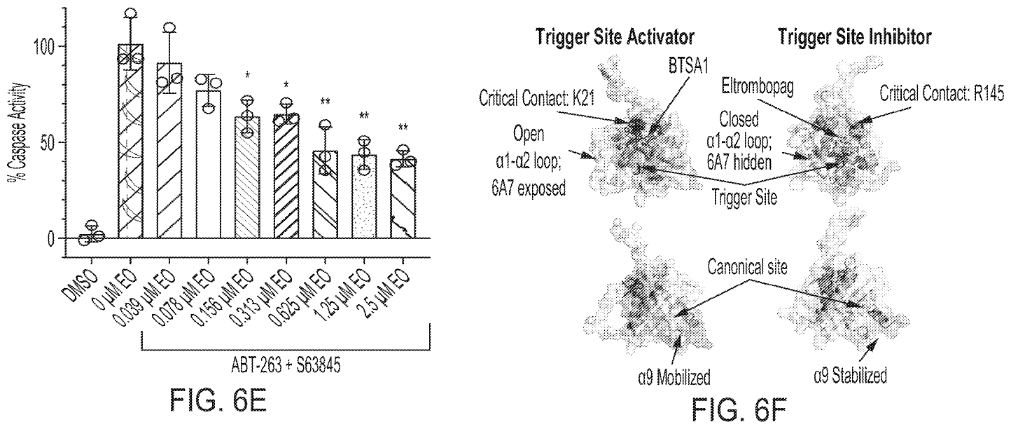

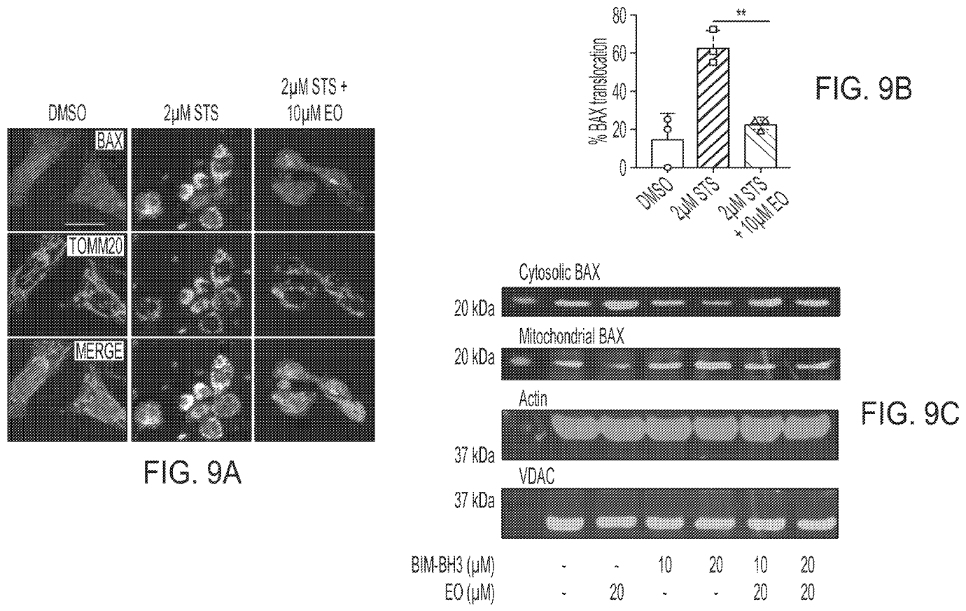

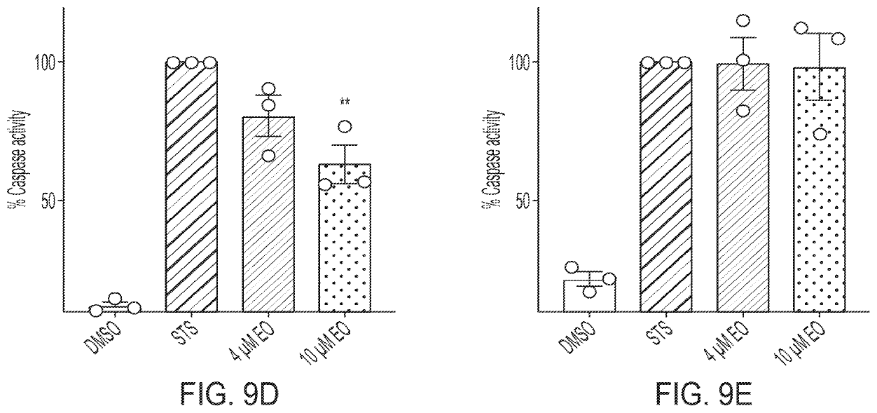

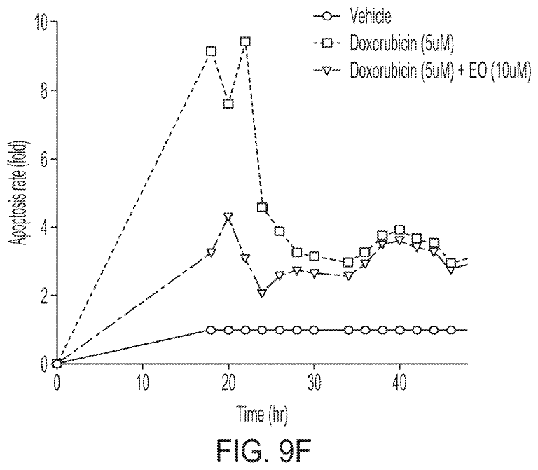

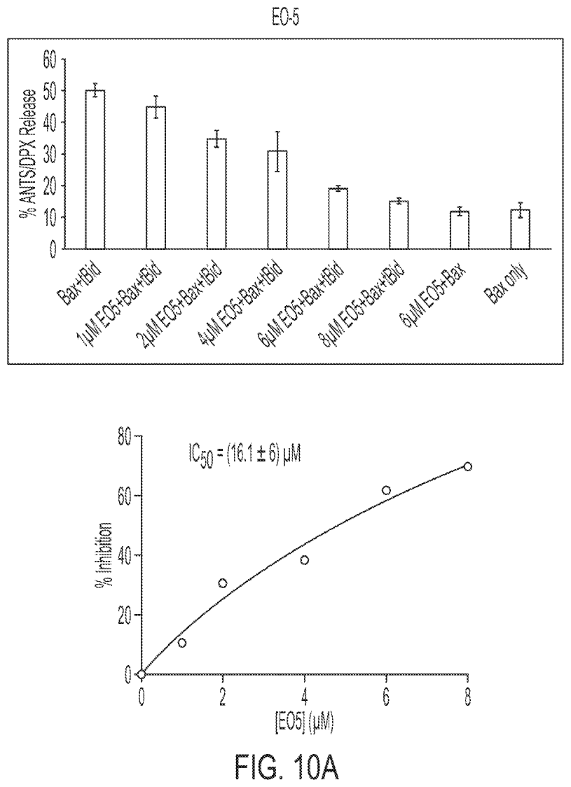

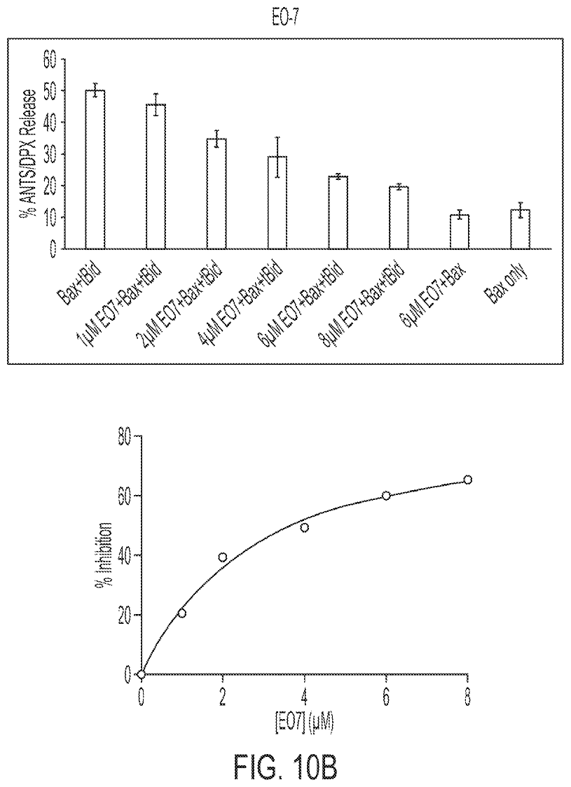

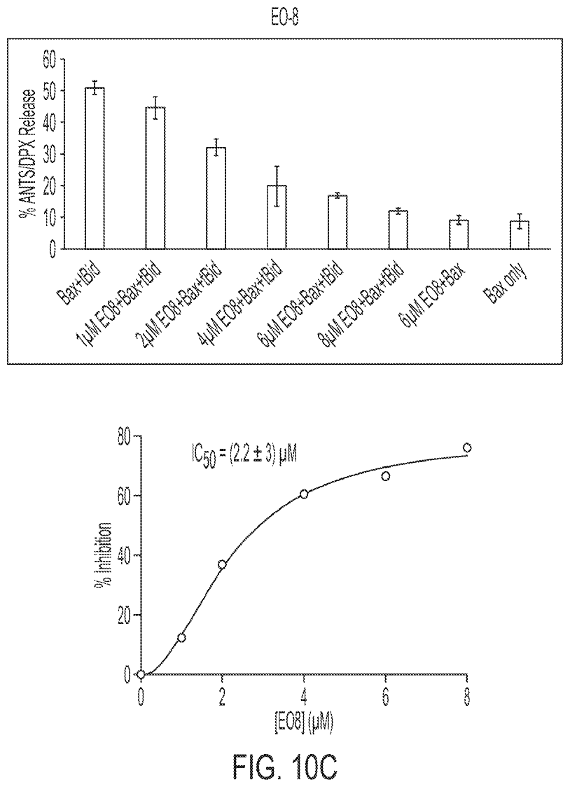

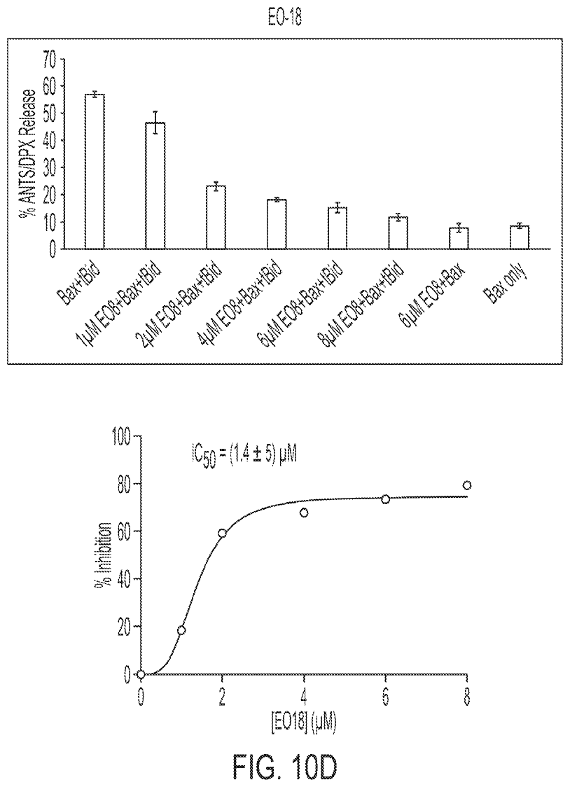

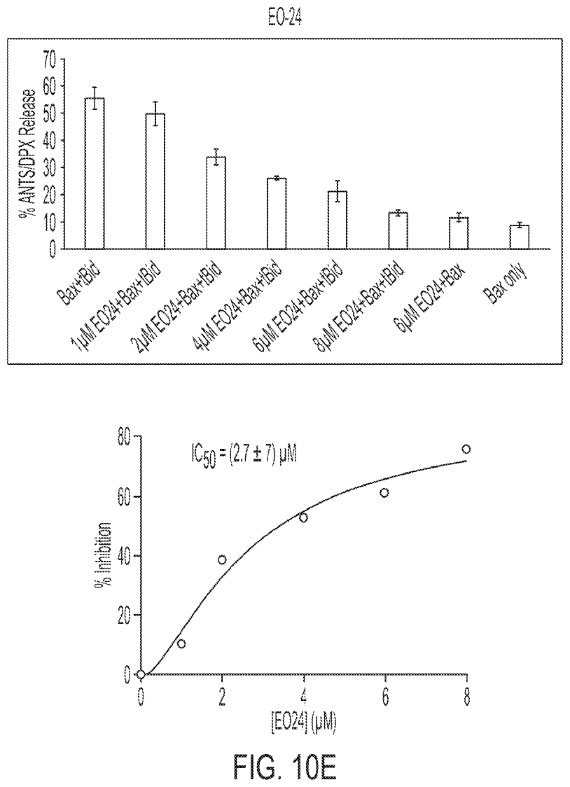

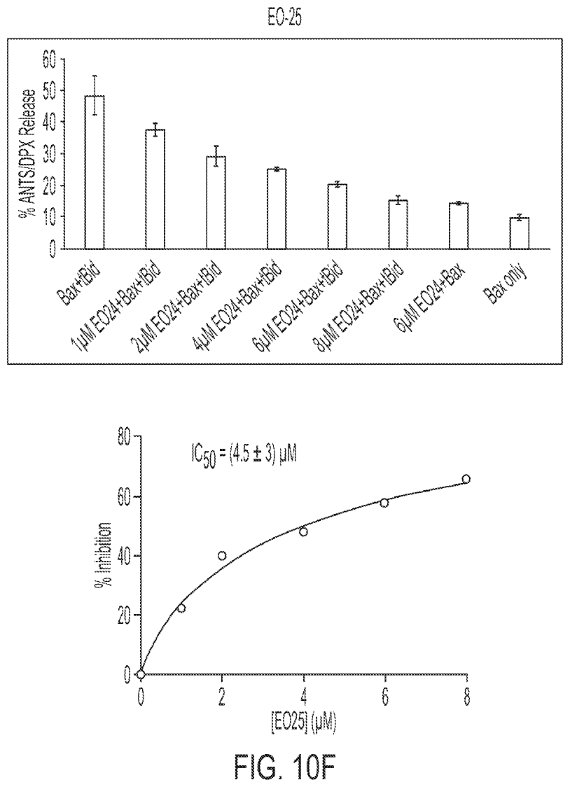

Thrombopoietin (THPO)-receptor agonist activity of EO is highly specific to the human and chimpanzee THPO-receptors, making mouse cell lines ideal for studying EO modulation of BAX-dependent activity independent of THPO-mediated effects (Erickson-Miller, C. L. et al. Stem Cells 27, 424-430 (2009)). First, mitochondrial cytochrome c release, a hallmark of BAX activation and BAX-dependent apoptosis, was evaluated. BIM-BH3 induced release of cytochrome c was significantly inhibited by EO in BAKKO (BAK^{-/-}) MEFs, providing direct evidence that EO can inhibit BAX-dependent cytochrome c release (FIG. 6a). EO had no such effect in BAXKO (BAX^{-/-}) MEFs, strongly supporting BAX specificity (FIG. 6b). BAXKO and BAKKO MEFs exhibited similar sensitivity to BIM-BH3 induced cytochrome c release. Mitochondrial translocation of cytosolic BAX upon treatment with either BIM BH3 or staurosporine (STS) in BAK KO MEFs was also evaluated. It was found that EO is capable of inhibiting BAX translocation (FIGS. 9a-c), consistent with in vitro results. Accordingly, EO inhibited STS-induced apoptosis mediated by caspase 3/7 activity in MEFs expressing only BAX, but it had no effect is MEFs expressing only BAK (FIGS. 9d-e).

To confirm whether this is linked to direct target engagement of BAX, Cellular Thermal Shift Assay (CETSA) was performed in BAKKO MEFs. CETSA showed that EO indeed binds BAX in cells by lowering its TM by 9° C. (FIG. 6c). Although decreased TM typically would imply destabilization of BAX by EO, previous studies have demonstrated that inactive BAX mutants can display dramatically reduced TM despite their resistance to activation by BH3-activators, highlighting a critical distinction between the controlled conformational changes of BAX activation and protein unfolding (Edwards, A. L. et al. Chem Biol 20, 888-902, (2013); Robin, A. Y., et al. Structure 26 1346-1359 (2018)). Furthermore, recombinant BAX displayed a comparable reduction in TM in the presence of EO, strongly supporting the CETSA observations.

In addition to inhibition of STS-induced apoptosis, it was additionally found that EO is capable of inhibiting apoptosis (cell death) of human IPSC cardiomyocytes induced by doxorubicin treatment (FIG. 9f), consistent with the protective functional role of BAX inhibition in doxorubicin-induced cardiotoxicity.

Next, whether EO is capable of rescuing cell death in cells expressing both BAX and BAK was determined. 3T3 cells were treated, a murine fibroblast cell line, with a combination of clinical BH3-mimetics ABT-263 (Navitoclax) and S63845, which only in combination caused significant cytotoxicity in fibroblast cells. Strikingly, cells treated with EO exhibited a dose-dependent rescue of cell viability as well as a corresponding significant reduction of apoptosis mediated by caspase 3/7 activity (FIGS. 6d and 6e).

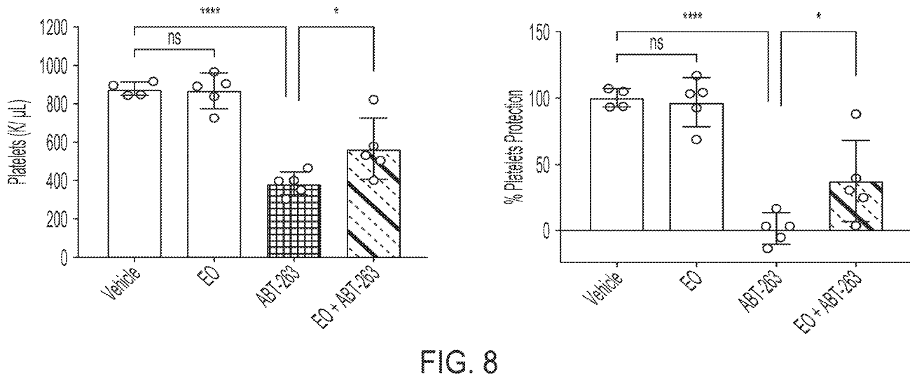

Additionally, whether EO can rescue thrombocytopenia induced by the activity of navitoclax was evaluated in mice. The use of navitoclax is limited by on-target toxicity triggering BAK/BAX-mediated platelet apoptosis (Zhang, H. et al. Cell Death Differ 14, 943-951 (2007)). While significant platelet loss was induced by navitoclax within 24 hr, co-administration of navitoclax with EO markedly inhibited platelet loss to acceptable levels (FIGS. 8a-b). This EO effect is distinct from its capacity to stimulate platelet production by differentiation of the megakaryocyte precursors and progenitor cells, which requires 5 days to begin (Erickson-Miller, C. L. et al. Stem Cells 27, 424-430 (2009); Jenkins, J. M. et al. Blood 109, 4739-4741 (2007)).

Elucidating the process of BAX activation, defined as the transformation of cytosolic BAX from inactive conformation into a deadly MOM oligomer, has been regarded as the “holy grail” of apoptosis research. Understanding this process not only will enable a deep understanding of the critical function of BAX in apoptosis signaling and related pathways (e.g., mitochondrial-driven necrosis, mitochondrial dynamics), but also will illuminate new targets and means to modulate BAX activity with drugs.

Here, the studies have identified EO, as a potent binder to the BAX trigger site and an effective BAX inhibitor. EO inhibits BAX activation by a novel two-fold mechanism. The data show that BAX inhibition by EO is dependent on the concentration of EO, BAX, and BH3-activators and that EO directly engages the BAX trigger site binding, consistent with a direct competitive mechanism. Furthermore, it was demonstrated that EO inhibits heat-induced translocation and activation of BAX, promotes stabilization of the α1-α2 loop in closed conformation and interaction with α6 and induces conformational changes associated with reduced BAX activity such as those observed at the α7/α4-α5 loop and canonical site-α9 interfaces. Thus, the data suggest a unique mechanism of BAX inhibition by EO that directly competes against BH3-only proteins binding to BAX and simultaneously promotes allosteric conformational changes that stabilize the inactive soluble BAX structure.

The N-terminal BAX trigger site has been established as an important binding site for BH3-only proteins and small molecule BAX activators that induce conformational changes throughout the BAX structure necessary for mitochondrial translocation, dimerization/oligomerization, and mitochondrial outer membrane permeabilization. This study provides further evidence that BH3-only proteins such as BID and BIM use the trigger site surface to induce BAX conformation activation. EO was identified by substructure similarity search of known small molecule BAX activators. Despite some similar structural features to BAX activators, EO engages the trigger site with a unique binding mode distinct from BAX activators, using hydrophobic interactions with a shallow hydrophobic groove formed by residues of α6, α1 and the closed α1-α2 loop, and hydrogen bonds with R143 and R134 of α6. This disclosure demonstrated for the first time that the BAX trigger site can also serve as a site of inhibition by small molecules and it can interfere with the early stages of BAX activation, before the disengagement of the α9 from the canonical groove and BAX mitochondrial translocation (FIG. 6f). Thus, the study offers a blueprint for rational design of a novel class of BAX inhibitors.

The data indicate a remarkable allosteric communication of BAX surfaces driven by interacting helices and BAX binding sites with functional plasticity that comes in agreement with previous studies. Allostery has been proposed with activator BH3 peptides and small molecules such as BAM7 and BTSA1 that engage the BAX trigger site and promote conformational changes leading to release of α9 from the opposite surface of BAX. Allosteric BAX inhibitors (BAIs) and fragments that sensitize BAX to activation bind to adjacent sites while exhibiting opposite functional effects by either preventing the exposure of the BAX BH3 helix or promoting the mobilization of α1-α2 loop from the core BAX structure, respectively (Garner, T. P., et al. Nat Chem Biol 15, 322-330 (2019); Pritz, J., et al. Nat Chem Biol 13, 961-967 (2017)). Furthermore, the BAX sensitizing fragments and the cytomegalovirus vMIA peptide compete for an identical binding site, yet the vMIA peptide inhibits BAX activity. Synthetic antibodies recognizing the N-terminal BAX trigger site also have served to inhibit BAX activation by blocking activator binding (Uchime, O. et al. J Biol Chem 291, 89-102, (2016)). Furthermore, the BCL-2 BH4 domain inhibits BAX by binding to a unique site on the surface of directly between the N-terminal trigger site and vMIA site (Barclay, L. A. et al. Mol Cell 57, 873-886, (2015)). Lastly, 3C10 antibody has an inhibitory effect to BAX by engaging the α1-α2 loop and favoring the allosteric sequestration of α9 in the canonical groove (Dengler, M. A., et al. Cell Rep. 27, 359-373 (2019); Iyer, S. et al. Nat Commun 7, 11734, (2016)). Therefore, it is possible that most, if not all, of the BAX surface could serve as a site of activation or inhibition given the appropriate interactions with a small molecule, peptide, or antibody/protein.

EO is the first FDA-approved molecule with the ability to modulate BAX activity directly. Interestingly, platelets isolated from patients treated with EO exhibited increased resistance to ABT-263 (Navitoclax) induced cell death (Mitchell, W. B. et al. Am J Hematol 89, (2014)). This was not observed in patients treated with the fusion protein thrombopoietin receptor agonist romiplostim. As increased platelet apoptosis contributes to certain forms of immune thrombocytopenia, it is possible that EO inhibition of BAX contributes to its therapeutic activity. The fact that it is a well-tolerated orally bioavailable molecule inspires confidence in the potential of BAX inhibition as a therapeutic strategy for diseases of aberrant cell death.

### Example 3

Discovery of novel BAX targeting small molecules offers an opportunity to develop them into drugs. Repurposing of clinically approved small molecules is a highly attractive approach, given the tremendous challenges of de-novo drug discovery and development (Pushpakom, S., et al. Nat Rev Drug Discov 18, 41-58 (2019)). While searching for direct BAX modulators based on the previous small molecule BAX activators (Gavathiotis, E., et al. Nat Chem Biol 8, 639-645 (2012); Reyna, D. E., et al. Cancer Cell 32 490-505 (2017)), EO was identified. EO is an FDA-approved thrombopoietin receptor agonist and iron chelator that is used to increase blood platelet counts due to chronic immune thrombocytopenia (Zhang, Y., et al. Clin. Ther. 33 1560-1576 (2011)). Despite some similarity with the BAX activators, EO proved instead to be a direct inhibitor of BAX.

This disclosure provides novel EO analogs having similar or better activity with EO as BAX inhibitors and cell death inhibitors. These analogs were designed based on the structural model of EO with BAX using NMR and molecular dynamics methods.

To design and develop novel EO analogs that are tailored for BAX inhibition, the NMR-based docked poses of EO within the BAX site were used to optimize hydrophobic and hydrogen bonding interactions. Therefore, analogs were designed to increase van der Waals and hydrogen bonding with the Glu44, Glu131, Thr135, and Arg134, using substitutions of the phenyl ring connected to the pyrazolone ring. Moreover, additional substitutions to the other two phenyl rings were designed to increase van der Waals and hydrogen bonding with residues such as Met20, Gly138, Ala24, Arg145, and Asp48, based on the NMR-based docked pose of EO.

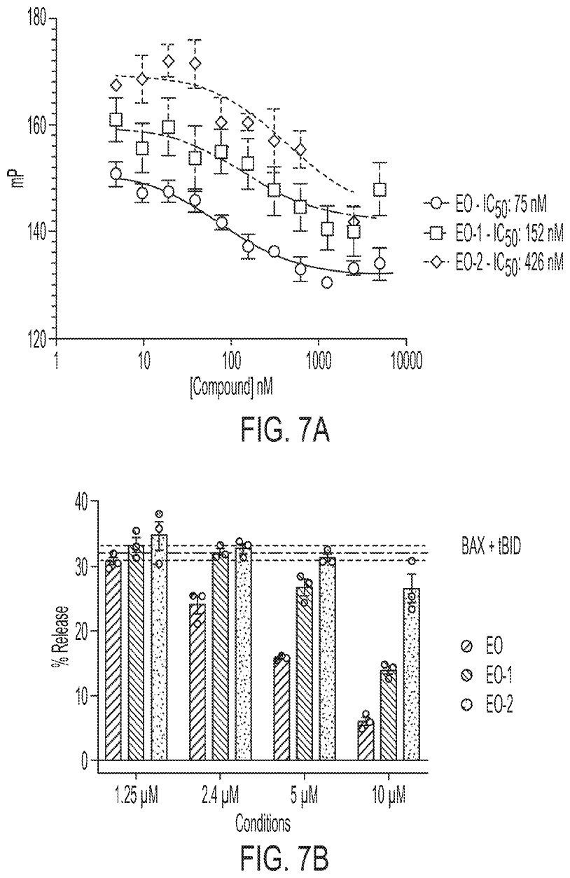

Finally, in order to avoid potential binding of iron with EO that can induce toxicity in vivo, the hydroxyl substitution of the phenyl ring was replaced with other groups that make additional contacts with BAX but have no ability to bind iron. For example, in several compounds such as EO-1, the hydroxyl is replaced by a methoxy or a methyl. EO-1 and EO-2 were synthesized and characterized and tested for binding to BAX and inhibition of BAX activation by tBID. Additional compounds are synthesized (as exemplified below) and evaluated for BAX inhibition in biochemical and cellular assays as well as in a liposome release assay (FIGS. 7a-b and 10a-f).

The compounds, as disclosed herein, can be represented by Formula (I):

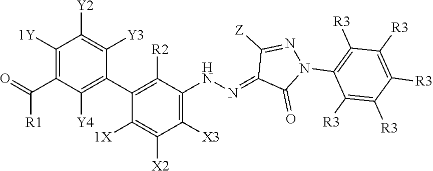

or
Formula (II):

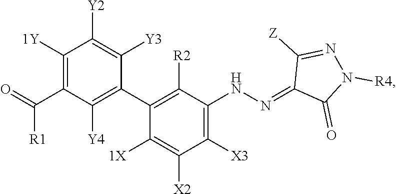

wherein:
R1 is selected from OH, O—CH₃, O—CH₂CH₃, O—CH(CH₃)₂, NH—CH₃, and NH—CH₂CH₃;
R2 is selected from H, F, Cl, CH₃, CF₃, OCH₃, CH₂CH₃, OCH₂CH₃;
R3 is selected from H, OH, CH₃, CF₃, NH₂, F, Cl, OCH₃, and NHCOCH₃;
X1, X2, or X3 is selected from H, F, Cl, OH, CH₃, CF₃, CH₂CH₃, OCH₃, OCH₂CH₃ and —CO—CH₃;
Y1, Y2, Y3, or Y4 is selected from H, F, Cl, CH₃, CF₃, OCH₃, and CH₂CH₃;
Z is selected from H, F, Cl, CH₃, CF₃, OCH₃, CH₂CH₃, CH₂NH₂, and CH₂CH₂NH₂;
R4 is selected from:

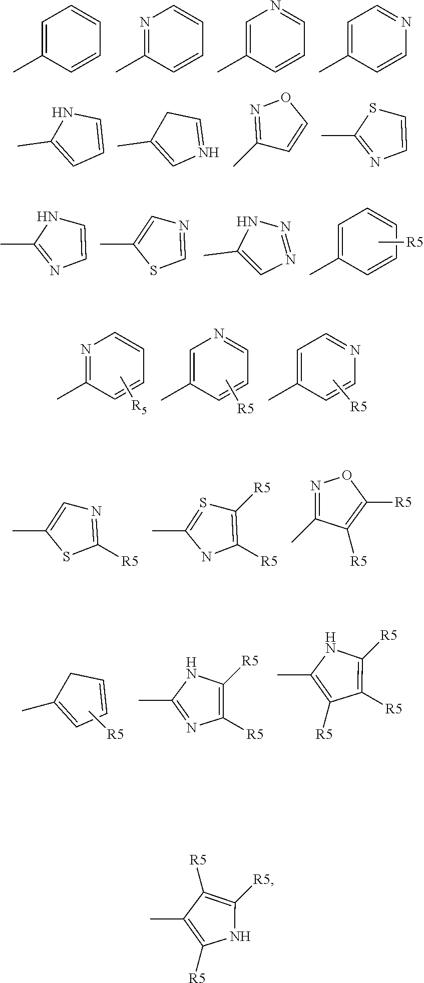

R5 is selected from H, OH, CH₃, CF₃, NH₂, F, Cl, OCH₃, and NHCOCH₃.

Representative compounds may include, without limitation,

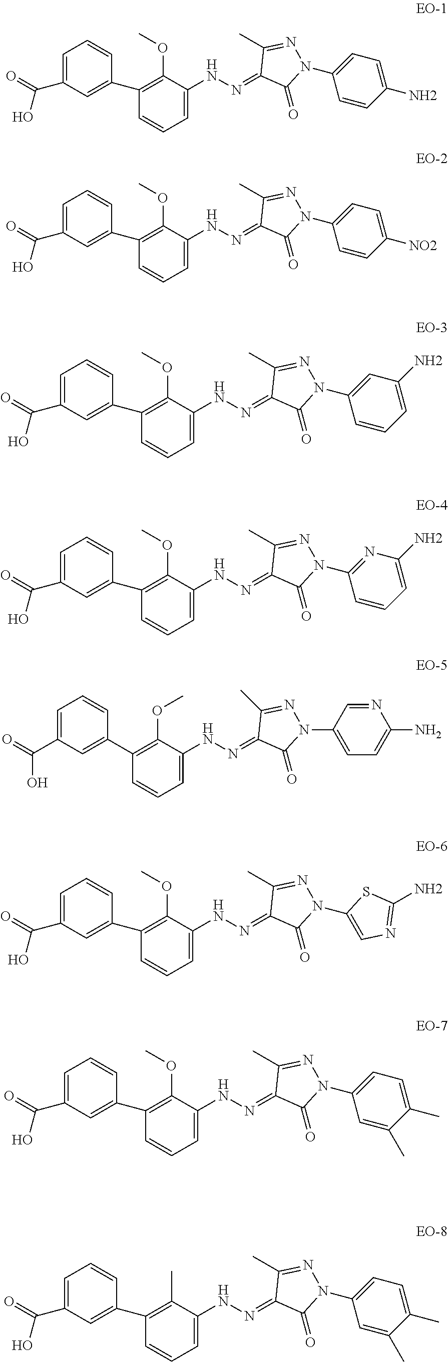

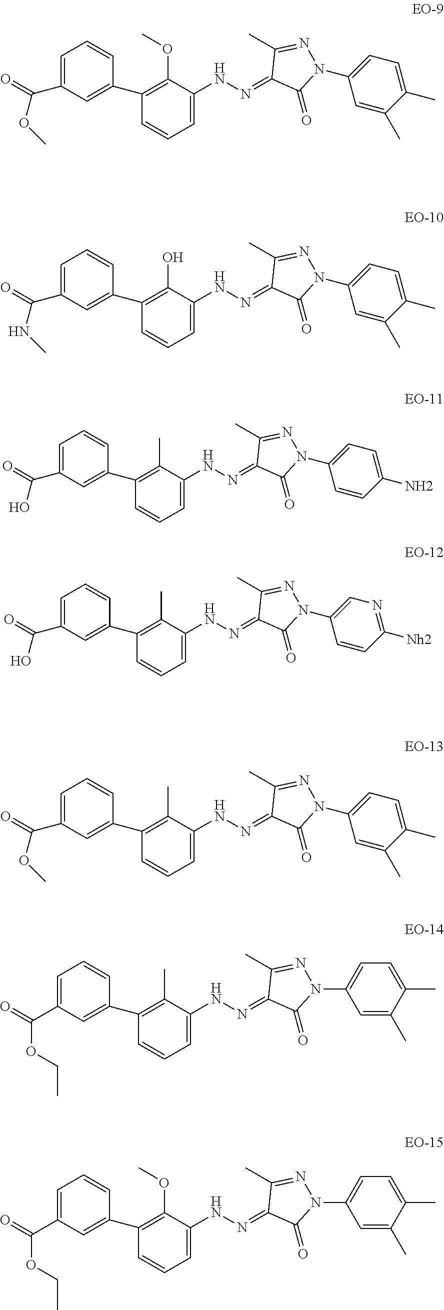

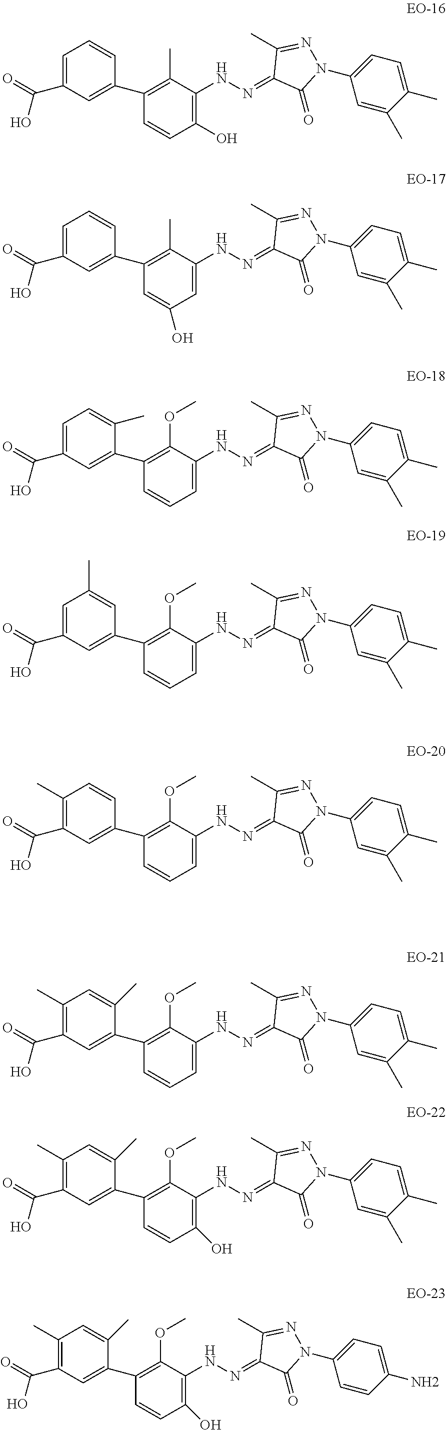

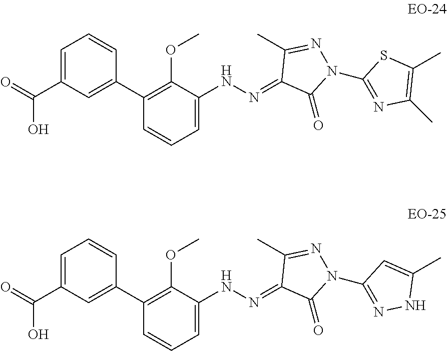 [SMILES: COc1c(N/N=C2/C(=O)N(c3nc(C)c(C)s3)N=C2C)cccc1-c1cccc(C(=O)O)c1 ; COc1c(N/N=C2/C(=O)N(c3cc(C)[nH]n3)N=C2C)cccc1-c1cccc(C(=O)O)c1]

### Example 4

The example synthetic routes for representative compounds are provided below.

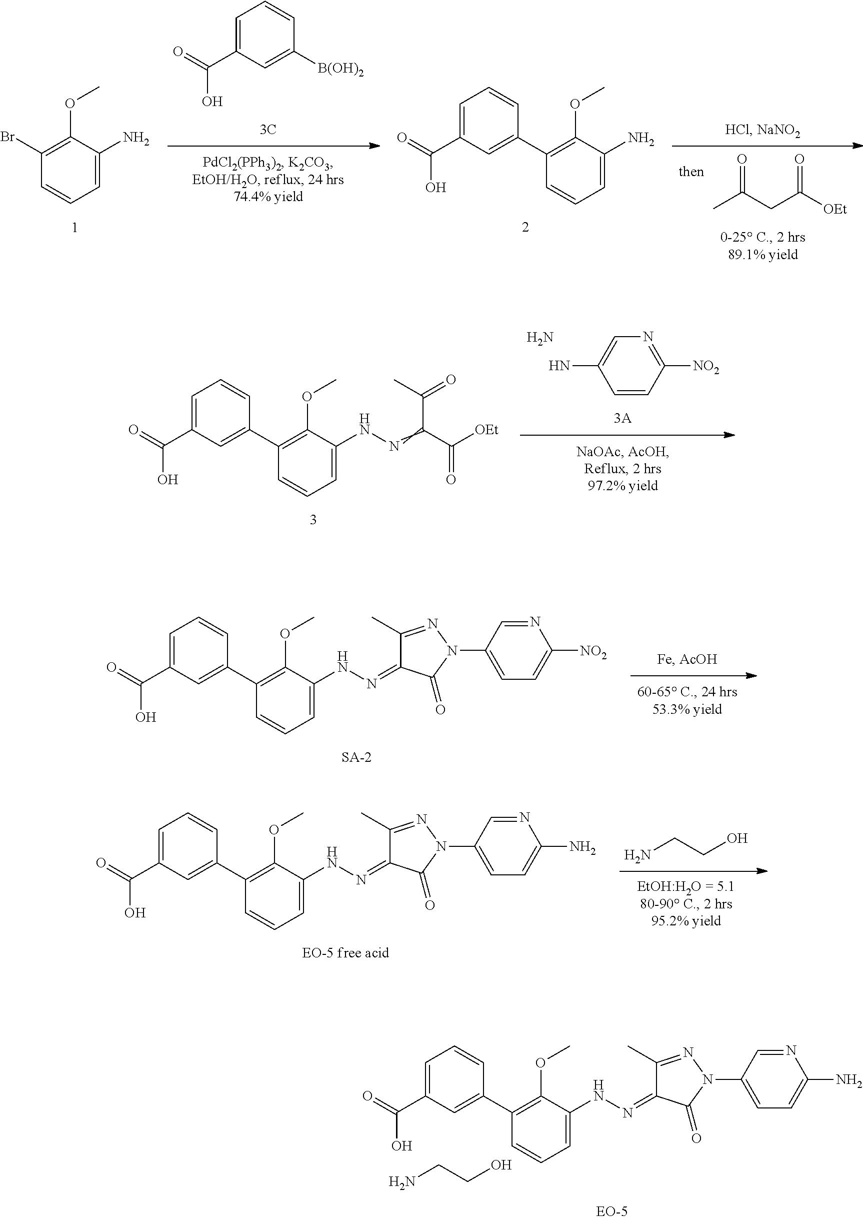

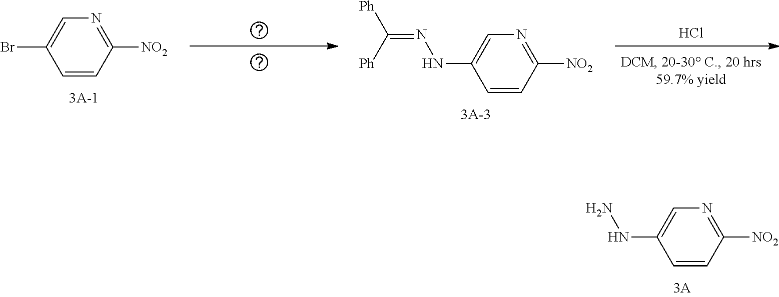 [SMILES: O=[N+]([O-])c1ccc(Br)cn1 ; O=[N+]([O-])c1ccc(NN=C(c2ccccc2)c2ccccc2)cn1 ; Cl ; NNc1ccc([N+](=O)[O-])nc1]

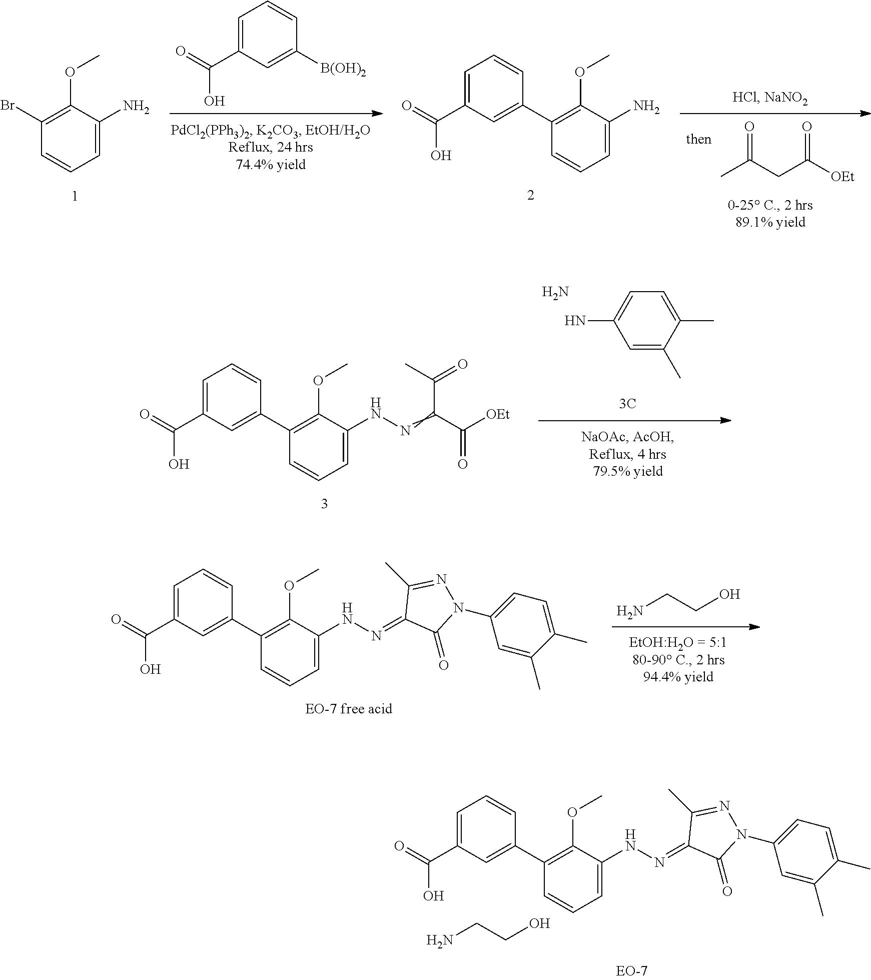

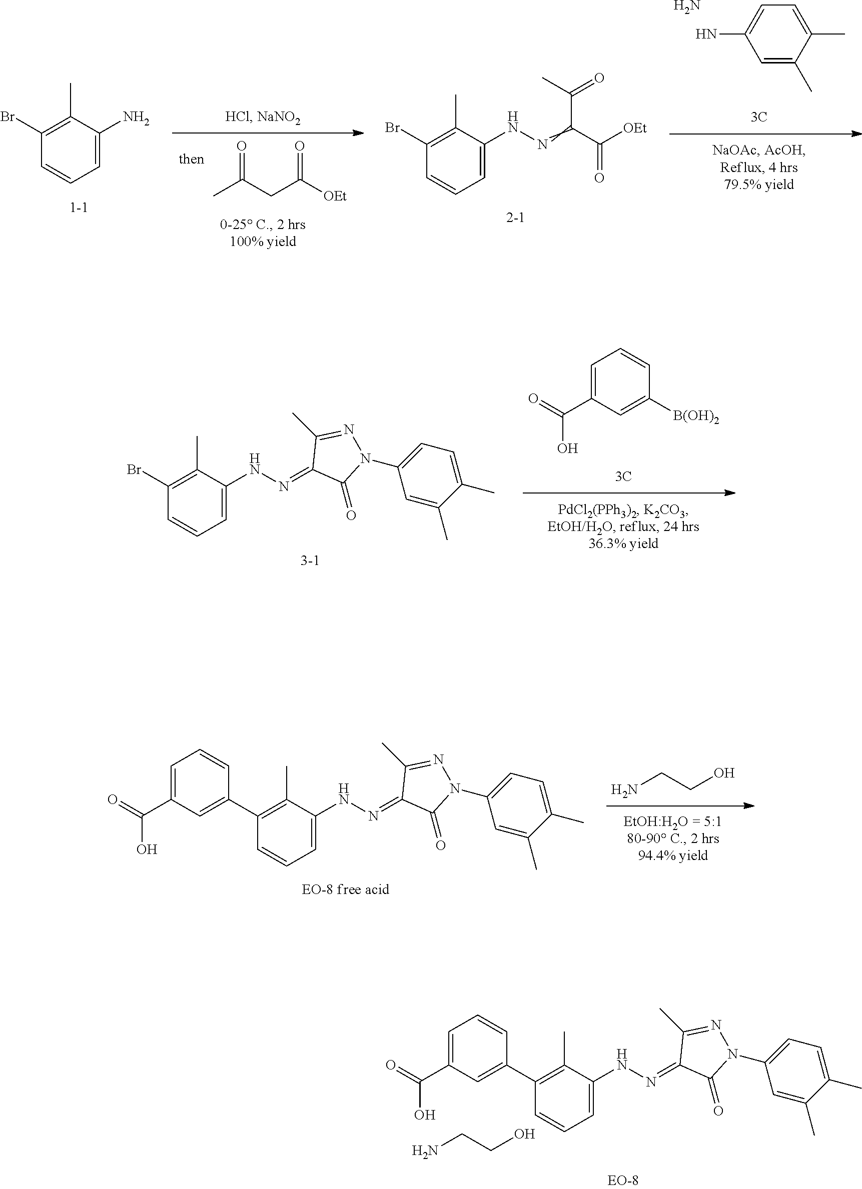

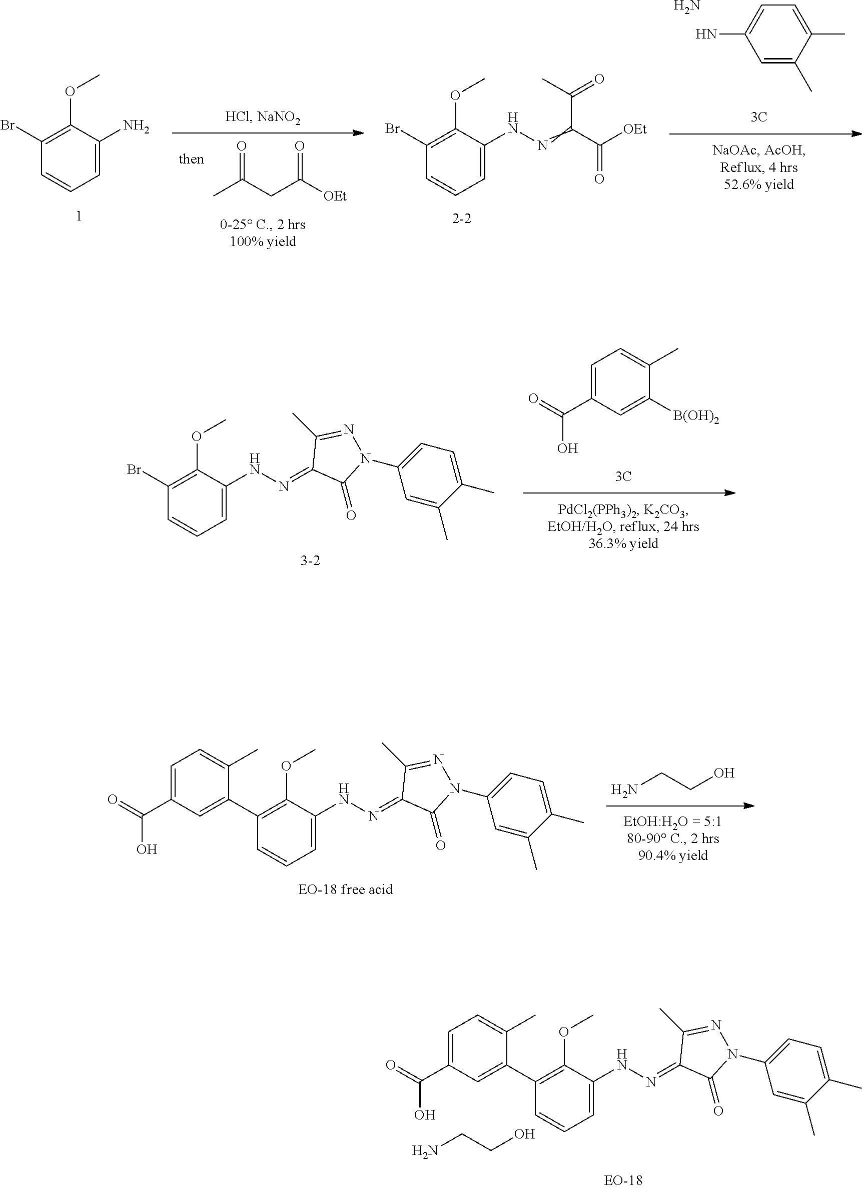

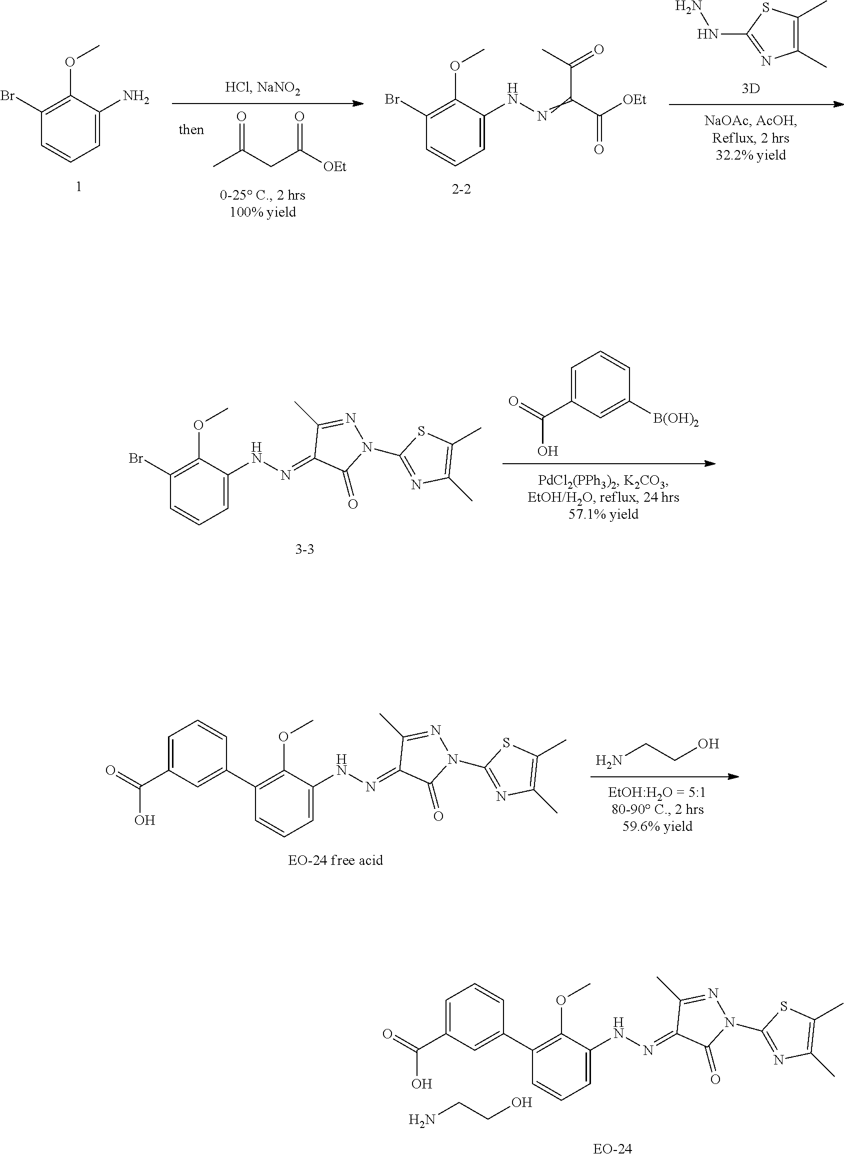

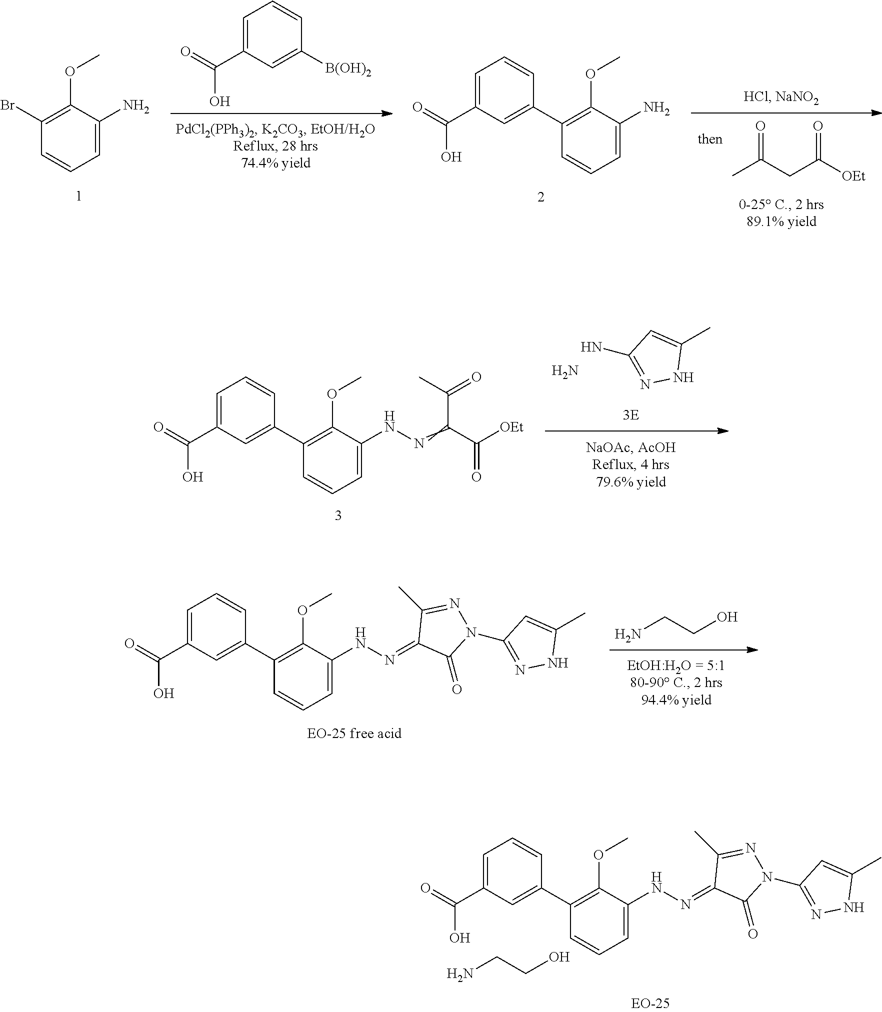

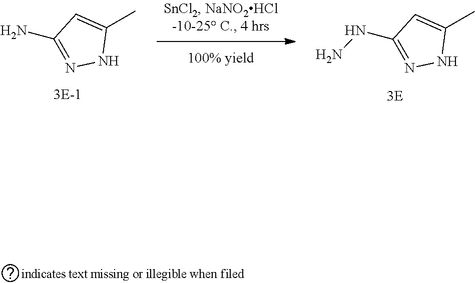

### General Procedure for Preparation of Compound 2

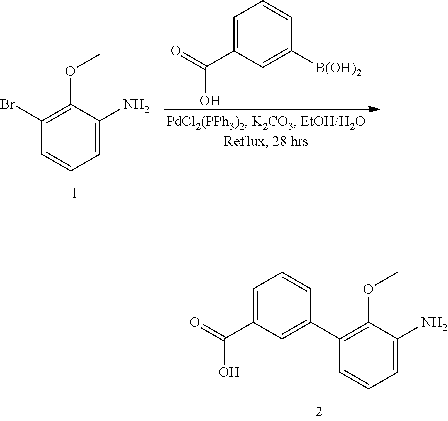

A mixture of 1 (12.0 g, 59.4 mmol, 1.00 eq), 3-boronobenzoic acid (11.8 g, 71.3 mmol, 1.20 eq), dichloropalladium; triphenylphosphane (1.25 g, 1.78 mmol, 0.0300 eq) and K₂CO₃ (20.5 g, 148 mmol, 2.50 eq) in EtOH (400 mL) and H₂O (80.0 mL) was heated to about reflux temperature and stirred the mixture for 2 hrs (inner temperature 80˜90° C.) under N₂ protect. The hot mixture was filtrate to remove the catalyst and wash cake with EtOH (50.0 mL). The clear filtrate was concentrated under vacuum. To the residue, H₂O (200 mL) and MeOH (200 mL) was added, acidified to pH 3-5 using hydrochloric acid (20.0 mL) and stirred for about 1 hr at 25° C. The slurry was filtered, washed with H₂O (80.0 mL) and concentrated to dryness at about 45° C. Crude product of compound 2 (10.8 g, 74.4% yield) as brown solid was obtained.

### General Procedure for Preparation of Compound 3

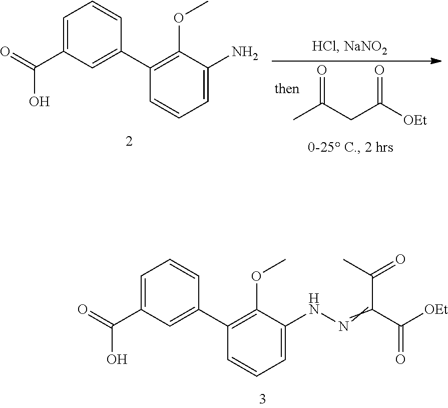

To a solution of 2 (5.00 g, 20.6 mmol, 1.00 eq) in hydrochloric acid (12.0 M, 8.50 mL, 4.96 eq in 90.0 mL MeOH) was added a solution of sodium nitrite (1.49 g, 21.6 mmol, 1.05 eq) in H₂O (400 mL) at about 0° C. to about 5° C. under stirring. The reaction mixture was stirred for about 15 minutes at about 5° C. The ethyl 3-oxobutanoate (2.70 g, 20.8 mmol, 2.62 mL, 1.01 eq) was added and the reaction mixture was stirred for about 15 minutes at about 0° C. to about 5° C. Solid sodium bicarbonate (15.0 g, 178 mmol, 8.69 eq) and EtOH (30.0 mL) was added and stirred for about 2 hrs at about 0° C. to about 5° C. The mixture solution was acidified pH to 3-5 with hydrochloric acid (12.0 M, 5.00 mL) and extracted with EtOAc (500 mL*3). Concentrated the mixture was to give crude product. Compound 3 (7.20 g, 89.1% yield) was obtained as light yellow solid.

General Procedure for Preparation of Compound 3A-2

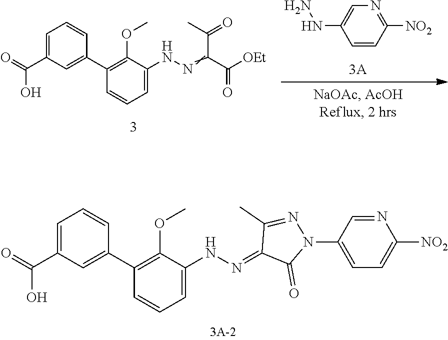 [SMILES: CCOC(=O)C(=NNc1cccc(-c2cccc(C(=O)O)c2)c1OC)C(C)=O ; NNc1ccc([N+](=O)[O-])nc1 ; COc1c(N/N=C2/C(=O)N(c3ccc([N+](=O)[O-])nc3)N=C2C)cccc1-c1cccc(C(=O)O)c1]

To a solution of 3 (500 mg, 1.30 mmol, 1.00 eq) and (6-nitro-3-pyridyl)hydrazine (300 mg, 1.57 mmol, 1.21 eq, HCl) in AcOH (4.00 mL) was added NaOAc (130 mg, 1.58 mmol, 1.22 eq) in one portion at 25° C. under N₂. Then stir the mixture at 120° C. for 2 hrs. TLC (DCM:MeOH=10:1, R_{f}=0.28) showed the reaction was completed. The reaction mixture was filtered and washed with H₂O (10.0 mL*2). The solid was triturated with H₂O (5.00 mL) for 1 hr at 20-30° C. The slurry was filtered, washed with H₂O (10.0 mL) and concentrated to dryness at about 45° C. Concentrated the mixture was to give crude product. Compound 3A-2 (0.60 g, 97.2% yield) was obtained as brown solid.

General Procedure for Preparation of Compound EO-5 Free Acid

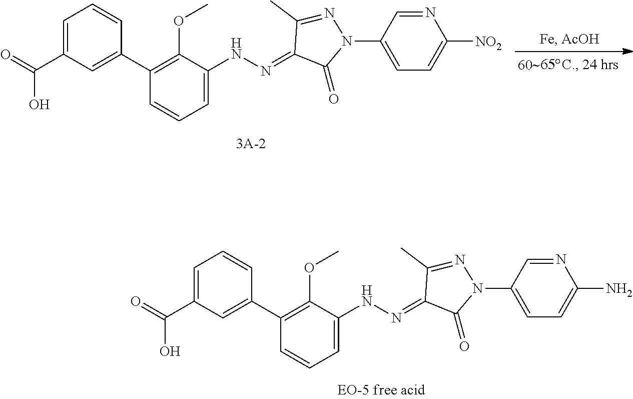 [SMILES: COc1c(N/N=C2/C(=O)N(c3ccc([N+](=O)[O-])nc3)N=C2C)cccc1-c1cccc(C(=O)O)c1 ; COc1c(N/N=C2/C(=O)N(c3ccc(N)nc3)N=C2C)cccc1-c1cccc(C(=O)O)c1]

To a solution of 3A-2 (300 mg, 632 μmol, 1.00 eq) in AcOH (6.30 g, 105 mmol, 6.00 mL, 166 eq) and EtOH (5.00 mL) was added Fe (200 mg, 3.58 mmol, 5.66 eq) in one portion at 25° C. under N₂. The mixture solution was stirred for 16 hrs at 60˜65° C. LCMS (EC722-44-P1A1, RT=0.576 min, M+1=445) showed product was detected. To reaction solution, H₂O (10.0 mL) was added and basified to pH 13˜14 using NaOH aqueous (1.00 M, 2.00 mL). The slurry was filtered to remove iron powder. The filtrate was acidified to pH 3˜4 using AcOH (5.00 mL), then filtered and washed with H₂O (5.00 mL). Compound EO-5 free acid (150 mg, 53.3% yield) was obtained as yellow solid.

LCMS: EC722-44-P1A1, product: RT=0.576 min, M+1=445

### General Procedure for Preparation of Compound EO-5

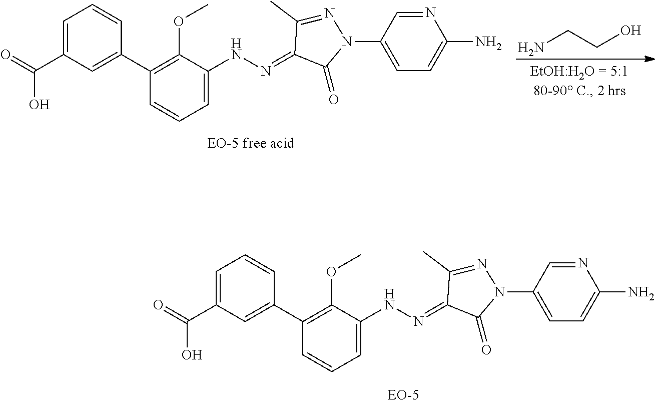

To a mixture of EO-5 free acid (60.0 mg, 135 μmol, 1.00 eq) and in EtOH:H2O=5:1 (5.00 mL) was added 2-aminoethanol (10.0 mg, 164 μmol, 9.90 uL, 1.21 eq) in one portion at 25° C. under N₂ protect. The reaction mixture was stirred for 2 hrs at 80° C. Concentrated the mixture was to give product. The product EO-5 (65.0 mg, 95.2% yield) was obtained as orange solid.

LCMS: EC722-64-P1A1, product: RT=1.50 mins, M+1=445

¹H NMR: EC722-64-P1A1 DMSO

δ 8.34 (s, 1H), 8.13 (s, 1H), 7.95-7.97 (d, J=7.38 Hz, 1H), 7.72-7.79 (m, 3H), 7.52-7.56 (t, J=7.63 Hz, 1H), 7.33-7.37 (m, 1H), 7.24-7.26 (d, J=7.00 Hz, 1H), 6.52-6.54 (d, J=7.00 Hz, 1H), 6.06 (s, 2H), 3.59-3.61 (t, J=5.32 Hz, 2H), 3.42-3.45 (m, 3H), 2.85-2.88 (t, J=5.32 Hz, 2H), 2.32 (s, 3H).

### General Procedure for Preparation of Compound 3A-3

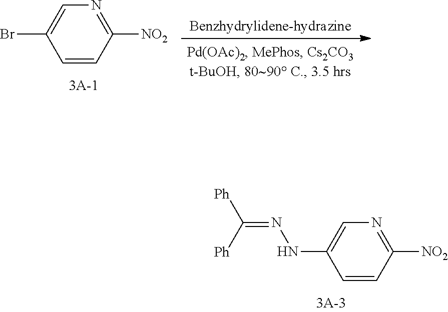 [SMILES: O=[N+]([O-])c1ccc(Br)cn1 ; O=[N+]([O-])c1ccc(NN=C(c2ccccc2)c2ccccc2)cn1]

A solution of Pd(OAc)₂ (55.3 mg, 246 μmol, 0.05 eq), Cs₂CO₃ (2.25 g, 6.90 mmol, 1.40 eq) and 2-Dicyclohexylphosphino-2′-methylbiphenyl (MePhos, 179 mg, 492 μmol, 0.100 eq) in t-BuOH (25.0 mL) was purged with N₂ for 30 mins. Then, 5-Bromo-2-nitro-pyridine (1.00 g, 4.93 mmol, 1.00 eq) and Benzhydrylidene-hydrazine (966 mg, 4.93 mmol, 1.00 eq) were successively added and the reaction mixture was heated under N₂ and vigorous stirring at 80° C. After 3 hrs, the reaction mixture was cooled to 25° C. and diluted with H₂O (80.0 mL). The precipitated product was collected by filtration, washed with petroleum/ethylacetate (1/1; v/v) and dried to yield 3A-3 as a brown solid (1.30 g, 81.6% yield).

### General Procedure for Preparation of Compound 3A

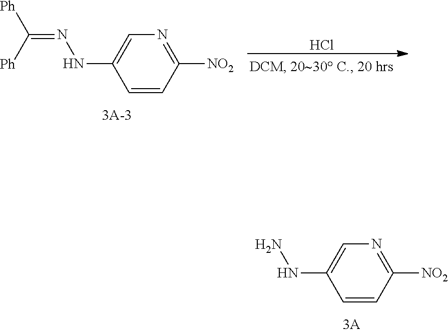 [SMILES: O=[N+]([O-])c1ccc(NN=C(c2ccccc2)c2ccccc2)cn1 ; Cl ; NNc1ccc([N+](=O)[O-])nc1]

To a solution of 3A-3 (700 mg, 2.20 mmol) in DCM (10.0 mL) was added HCl (12.0 M, 4.00 mL) and the reaction mixture was stirred for 20 hrs at 30° C. TLC (PE:EA=3:1) showed raw material was consumed fully (R_{f}=0.4) and new point was detected (DCM:MeOH=20:1, R_{f}=0.15). The slurry was filtered and the wet cake was triturated with DCM (10.0 mL*2). Crude product of compound 3A (250 mg, 59.7% yield) was obtained as light yellow solid.

### General Procedure for Preparation of Compound EO-7Free Acid

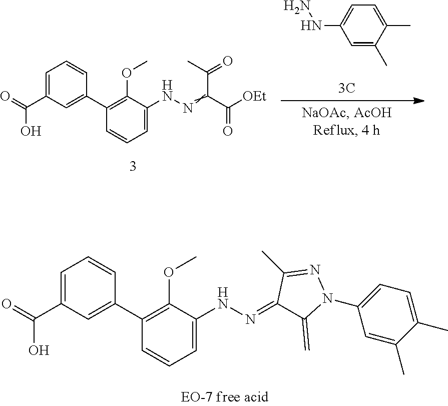

To a solution of 3 (430 mg, 1.12 mmol, 1.00 eq) and (6-nitro-3-pyridyl)hydrazine (241 mg, 1.40 mmol, 1.25 eq, HCl) in AcOH (10.0 mL) was added NaOAc (110 mg, 1.34 mmol, 1.20 eq) in one portion at 25° C. under N₂. Then stir the mixture at 120° C. for 2 hrs and another 1 hr at 25° C. LCMS showed the reaction was completed (EC722-11-P1A3, RT=1.75 min, M+1=475). The reaction mixture was filtered and washed with H₂O (10.0 mL). The solid was triturated with MeOH (5.00 mL) for 2 hrs at 20-25° C. The slurry was filtered, washed with H₂O (10.0 mL) and concentrated to dryness at about 45° C. Compound EO-7 free acid (300 mg, 657 μmol, 58.6% yield) was obtained as yellow solid.

LCMS: EC722-11-P1A3, product: RT=1.75 min, M+1=457

### General Procedure for Preparation of Compound EO-7

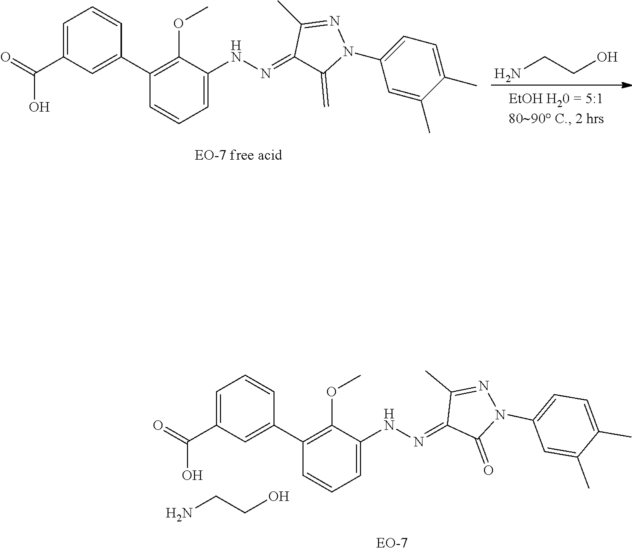

To a mixture of EO-7 free acid (80.0 mg, 175 μmol, 1.00 eq) and in EtOH:H2O=5:1 (5.00 mL) was added 2-aminoethanol (13.0 mg, 213 μmol, 12.9 uL, 1.21 eq) in one portion at 25° C. under N₂ protect. The reaction mixture was stirred for 2 hrs at 80° C. Concentrated the mixture was to give product. The product EO-7 (82.0 mg, 90.1% yield) was obtained as light yellow solid.

LCMS: EC722-30-P1B1, product: RT=1.75 min, M+1=457

¹H NMR: EC722-30-P1A2 DMSO

δ 8.12 (s, 1H), 7.92-7.94 (d, J=7.13 Hz, 1H), 7.67-7.71 (m, 2H), 7.62-7.64 (m, 2H), 7.45-7.49 (t, J=7.44 Hz, 1H), 7.28-7.35 (m, 1H), 7.19-7.21 (m, 2H), 3.58-3.61 (t, J=5.32 Hz, 3H), 3.48 (s, 2H), 2.84-2.87 (t, J=5.32 Hz, 2H), 2.33 (s, 3H), 2.26 (s, 3H), 2.22 (s, 3H).

### General Procedure for Preparation of Compound 2-1

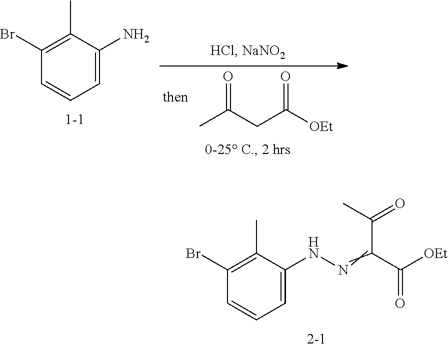

To a solution of 1-1 (1.00 g, 5.37 mmol, 1.00 eq) in HCl (1.00 M, 21.5 mL) was added a solution of sodium niteite (389 mg, 5.64 mmol, 1.05 eq) at about 0° C. The reaction stirred at 0° C. for 15 mins. Then add ethyl 3-oxobutanoate (699 mg, 5.37 mmol, 679 uL, 1.00 eq) to the mixture at 0° C. Stir the mixture at 0-5° C. for 15 mins. Add NaHCO₃ (1.49 g, 17.7 mmol, 689 uL, 3.30 eq) and EtOH (20.0 mL) to the mixture at 0° C. Stir the mixture at 25° C. for 2 hrs. HPLC showed the reaction was completed. The product was detected (RT=2.99 mins, 3.14 mins). The mixture was cooled to 25° C. The mixture was filtered, washed with water (10.0 mL). Concentrated the mixture was to give crude product. Compound 2-1 (3.20 g, 100% yield) was obtained as yellow solid.

### General Procedure for Preparation of Compound 3-1

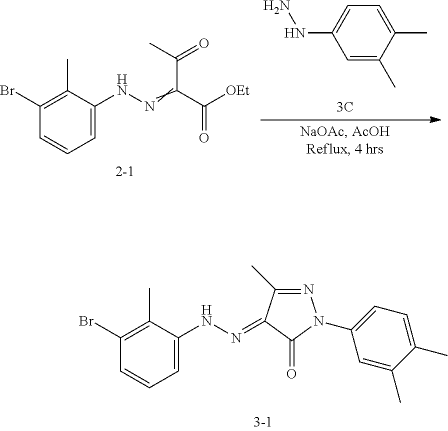

To a solution of 2-1 (1.65 g, 5.04 mmol, 1.00 eq) and (3,4-dimethylphenyl) hydrazine (858 mg, 6.30 mmol, 1.25 eq) in AcOH (30.0 mL) was added NaOAc (484 mg, 5.90 mmol, 1.17 eq) in one portion at 25° C. under N₂. And then stir the mixture at 120° C. for 3 hrs. HPLC showed the reaction was completed. The product was detected (RT=1.92 mins). The mixture was cooled to 25° C. The mixture was filtered, washed with water (10.0 mL). Concentrated the mixture was to give crude product. Compound 3-1 (1.66 g, 79.5% yield) was obtained as red solid.

LCMS: EC457-16-P4A, RT=1.92 mins, M+1=471

### General Procedure for Preparation of Compound EO-8 Free Acid

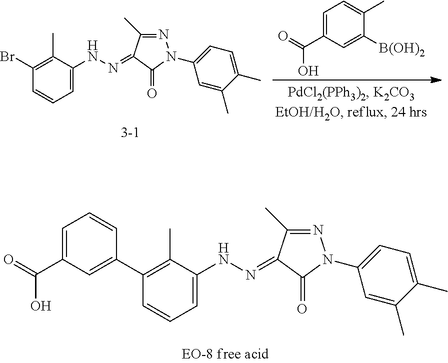 [SMILES: CC1=NN(c2ccc(C)c(C)c2)C(=O)/C1=N/Nc1cccc(Br)c1C ; Cc1ccc(C(=O)O)cc1B(O)O ; CC1=NN(c2ccc(C)c(C)c2)C(=O)/C1=N/Nc1cccc(-c2cccc(C(=O)O)c2)c1C]

To a mixture of 3-1 (1.00 g, 2.50 mmol, 1.00 eq) and 3-boronobenzoic acid (498 mg, 3.01 mmol, 1.20 eq) in EtOH (25.0 mL) and H₂O (5.00 mL) was added PdCl₂(PPh₃)₂ (52.7 mg, 75.1 μmol, 0.0300 eq) and K2CO3 (865 mg, 6.26 mmol, 2.50 eq) in one portion at 25° C. under N₂. The mixture was stirred at 80° C. stirred for 20 hrs. LCMS showed the reaction was completed. The product was detected (RT=2.60 mins). The hot mixture was filtered to remove the catalyst. The clear filtrate was concentrated under vacuum. To the residue, water (10.0 mL) and methanol (10.0 mL) was added. Acidified to pH 3-5 using hydrochloric acid. The mixture was stirred for about 30 mins at about 25° C. The slurry was filtered, washed with n-hexane (10.0 mL). Dried in an oven at about 55° C. Compound EO-8 free acid (500 mg, 36.3% yield) was obtained as red solid.

LCMS: EC457-17-P1F, RT=2.60 mins, M+1=471

### General Procedure for Preparation of Compound EO-8

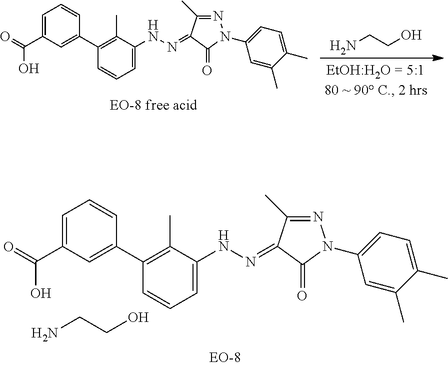

To a mixture of EO-8 free acid (80.0 mg, 181 μmol, 1.00 eq) and in EtOH:H2O=5:1 (5.00 mL) was added 2-aminoethanol (13.3 mg, 217 μmol, 13.1 uL, 1.20 eq) in one portion at 25° C. under. HPLC showed the reaction was completed. The product was detected (RT=1.92 mins). The mixture was stirred at 80° C. for 2 hrs. Concentrated the mixture was to give product. The product EO-8 (86.0 mg, 94.4% yield) was obtained as yellow oil.

LCMS: EC457-42-P1E, product: RT=1.77 mins, M+1=441

¹H NMR: EC457-42-P2C DMSO

δ 7.73-7.85 (d, 1H), 7.48-7.69 (m, 1H), 7.15-7.45 (m, 3H), 7.02 (s, 2H), 3.31-3.59 (t, 2H), 2.83-2.86 (t, 2H), 2.32 (s, 3H), 2.24 (s, 3H), 2.20 (s, 3H)

### General Procedure for Preparation of Compound 2-2

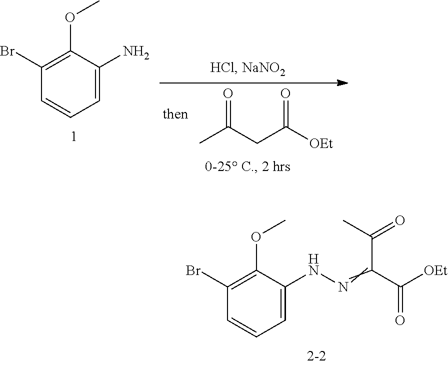

To a solution of 1 (2.00 g, 9.90 mmol, 1.00 eq) in HCl (1.00 M, 40.0 mL) was added a solution of sodium niteite (0.720 g, 10.4 mmol, 1.05 eq) at about 0° C. The reaction stirred at 0° C. for 15 mins. Then add ethyl 3-oxobutanoate (1.29 g, 9.90 mmol, 1.25 mL, 1.00 eq) to the mixture at 0° C. Stir the mixture at 0-5° C. for 15 mins. Add NaHCO₃ (2.75 g, 32.7 mmol, 1.27 mL, 3.31 eq) and EtOH (40.0 mL) to the mixture at 0° C. Stir the mixture at 25° C. for 2 hrs. LCMS showed the reaction was completed and the product was detected (RT=0.670 min, RT=0.702 min). The mixture was cooled to 25° C. The mixture was filtered, washed with water (10.0 mL). Concentrated the mixture was to give crude product. Compound 2-2 (2.80 g, 100% yield) was obtained as yellow solid.

HPLC: EC457-10-P2A, RT=2.847 mins, 2.979 mins

### General Procedure for Preparation of Compound 3-1

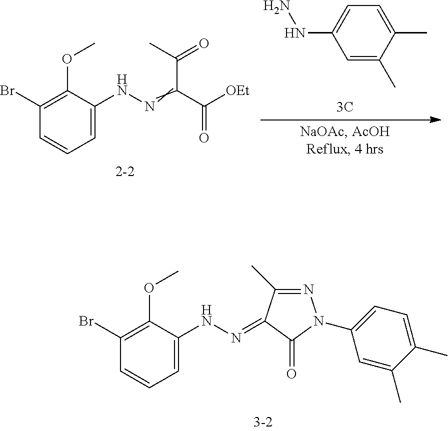

To a solution of 2-2 (500 mg, 1.46 mmol, 1.00 eq) and 3C (248 mg, 1.82 mmol, 1.25 eq) in AcOH (10.0 mL) was added NaOAc (140 mg, 1.71 mmol, 1.17 eq) in one portion at 25° C. under N₂ protect. And then stir the mixture at 120° C. for 2 hrs. LCMS showed desired product was detected (EC557-8-P1B1, RT=0.857 min, M+1=417). The mixture was cooled to 25° C. The mixture was filtered, washed with water (10.0 mL). Crude product of compound 3-2 (1.00 g, 82.6% yield) was obtained as yellow solid.

LCMS: EC557-8-P1B1, product: RT=0.857 mins, M+1=417

### General Procedure for Preparation of Compound EO-18 Free Acid

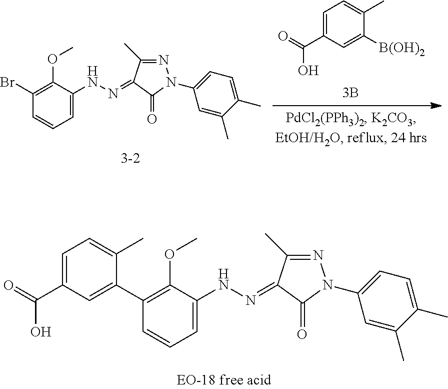 [SMILES: COc1c(Br)cccc1N/N=C1/C(=O)N(c2ccc(C)c(C)c2)N=C1C ; Cc1ccc(C(=O)O)cc1B(O)O ; COc1c(N/N=C2/C(=O)N(c3ccc(C)c(C)c3)N=C2C)cccc1-c1cc(C(=O)O)ccc1C]

To a mixture of 3-2 (800 mg, 1.93 mmol, 1.00 eq) and 3-boronobenzoic acid (416 mg, 2.31 mmol, 1.20 eq) in EtOH (20.0 mL) and H₂O (4.00 mL) was added PdCl₂(PPh₃)₂ (40.5 mg, 57.8 μmol, 0.0300 eq) and K₂CO₃ (666 mg, 4.82 mmol, 2.50 eq) in one portion at 25° C. under N₂. The mixture was stirred at 80° C. stirred for 20 hrs. LCMS showed the reaction was completed. The product was detected (RT=2.59 mins). The hot mixture was filtered to remove the catalyst. The clear filtrate was concentrated under vacuum. To the residue, water (10.0 mL) and methanol (10.0 mL) was added. Acidified to pH 3-5 using hydrochloric acid. The mixture was stirred for about 30 mins at about 25° C. The slurry was filtered, washed with n-hexane (10.0 mL). Dried in an oven at about 55° C. Compound EO-18 free acid (500 mg, 36.3% yield) was obtained as red solid.

LCMS: EC457-14-P2B, RT=2.59 mins, M+1=471

### General Procedure for Preparation of Compound EO-18

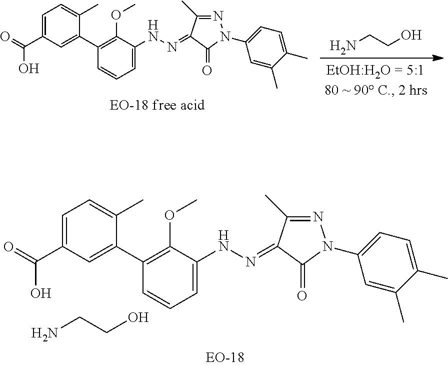

To a solution of EO-18 freed acid (80.0 mg, 170 μmol, 1.00 eq) and in EtOH:H2O=5:1 (5.00 mL) was added 2-aminoethanol (12.4 mg, 204 μmol, 12.3 uL, 1.20 eq) in one portion at 25° C. under. LCMS showed the reaction was completed and the product was detected (RT=1.76 mins). The mixture was stirred at 80° C. for 2 hrs. Concentrated the mixture was to give product. The product EO-18 (77.0 mg, 90.4% yield) was obtained as yellow oil.

LCMS: EC457-44-P1E, product: RT=1.76 mins, M+1=471

¹H NMR: EC457-44-P2A DMSO

δ 8.11 (s, 1H), 7.71-7.93 (d, 2H), 7.15-7.45 (d, 1H), 7.63-7.71 (d, 1H), 7.40-7.49 (d, 2H), 7.32 (s, 1H), 7.21 (s, 1H), 3.56-3.58 (t, 2H), 3.39 (s, 3H), 2.84-2.82 (t, 2H), 2.30 (s, 3H), 2.21 (s, 9H)

### General Procedure for Preparation of Compound 3-3

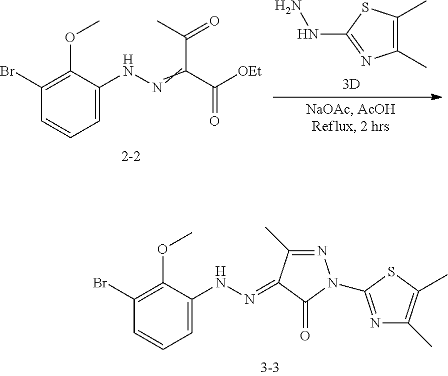

To a solution of 2-2 (1.05 g, 3.06 mmol, 1.00 eq) and (4,5-dimethylthiazol-2-yl)hydrazine (547 mg, 3.82 mmol, 1.25 eq) in AcOH (20.0 mL) was added NaOAc (484 mg, 5.90 mmol, 1.17 eq) in one portion at 25° C. under N₂. And then stir the mixture at 120° C. for 3 hrs. LCMS showed the reaction was completed. The product was detected (RT=2.26 mins). The mixture was cooled to 25° C. The mixture filtered, washed with water (10.0 mL). Concentrated the mixture was to give crude product. Compound 3-3 (520 mg, 32.2% yield) was obtained as yellow solid.

LCMS: EC457-17-P1F, RT=2.26 mins, Ms+1=471

### General Procedure for Preparation of Compound EO-24Free Acid

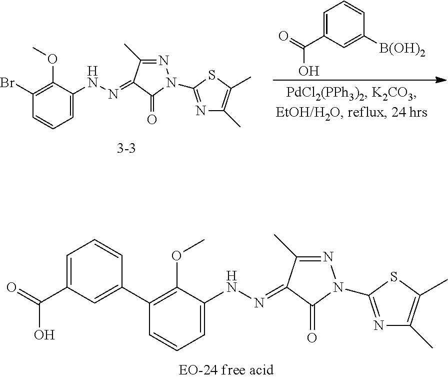 [SMILES: COc1c(Br)cccc1N/N=C1/C(=O)N(c2nc(C)c(C)s2)N=C1C ; O=C(O)c1cccc(B(O)O)c1 ; COc1c(N/N=C2/C(=O)N(c3nc(C)c(C)s3)N=C2C)cccc1-c1cccc(C(=O)O)c1]

To a mixture of 3-3 (400 mg, 947 μmol, 1.00 eq) and 3-boronobenzoic acid (188 mg, 1.14 mmol, 1.20 eq) in EtOH (10.0 mL) and H₂O (4.00 mL) was added PdCl₂(PPh₃)₂ (19.9 mg, 28.4 μmol, 0.0300 eq) and K₂CO₃ (327 mg, 2.37 mmol, 2.50 eq) in one portion at 25° C. under N₂. The mixture was stirred at 80° C. stirred for 20 hrs. LCMS showed the reaction was completed. The product was detected (RT=2.75 mins). The hot mixture was filtered to remove the catalyst. The clear filtrate was concentrated under vacuum. To the residue, water (10.0 mL) and methanol (10.0 mL) was added. Acidified to pH 3˜5 using hydrochloric acid. The mixture was stirred for about 30 mins at about 25° C. The slurry was filtered, washed with n-hexane (10.0 mL). Dried in an oven at about 55° C. Compound EO-24 free acid (30.0 mg, 67.1% yield) was obtained as red solid.

LCMS: EC457-24-P1B, RT=2.75 mins, M+1=471

General Procedure for Preparation of Compound EO-24

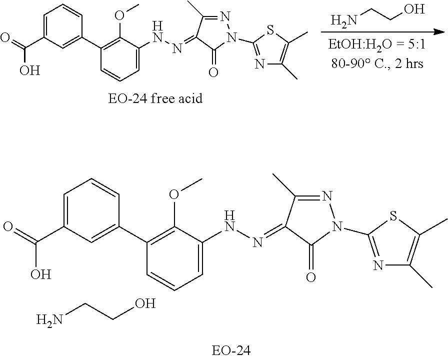

To a mixture of EO-24 free acid (80.0 mg, 172 μmol, 1.00 eq) and in EtOH:H2O=5:1 (5.00 mL) was added 2-aminoethanol (13.0 mg, 212 μmol, 12.8 uL, 1.23 eq) in one portion at 25° C. under. LCMS (EC722-31-P1B1) showed product was detected (RT=2.12 mins, M+1=464). The mixture was stirred at 80° C. for 2 hrs. Concentrated the mixture was to give product. The product EO-24 (86.0 mg, 89.6% yield) was obtained as yellow oil.

LCMS: EC722-31-P1B1, RT=2.12 mins, M+1=464

¹H NMR: EC722-31-P2A1 DMSO

δ 8.10 (s, 1H), 7.92-7.94 (d, J=7.75 Hz, 1H), 7.72-7.74 (d, J=7.88 Hz, 1H), 7.52-7.56 (m, 2H), 7.13-7.17 (m, 2H), 3.75 (s, 2H), 3.57-3.59 (m, 2H), 2.85-2.87 (m, 2H), 2.36 (s, 3H), 2.26 (s, 3H), 2.18 (s, 3H)

### General Procedure for Preparation of Compound 3E

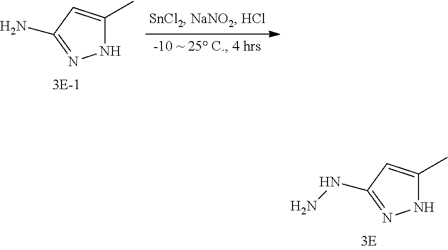

To a solution of 3E-1 (1.00 g, 10.3 mmol, 1.00 eq) and NaNO₂ (710 mg, 10.3 mmol, 1.00 eq) in HCl (6.00 mL) was added SnCl₂ (279 mg, 3.41 mmol, 1.17 eq) in HCl (6.00 mL) at −10° C. And then stir the mixture at 25° C. for 3 hrs. TLC (DCM/MeOH=1/1, R_{f}(3E-1)=0.70) showed that the reaction was complete. The crude product was added 5.00 mL MTBE and stirred for 2 hrs. The mixture was concentrated to give the compound. Compound 3E (4.30 g) was obtained as yellow solid.

### General Procedure for Preparation of Compound EO-25 Free Acid

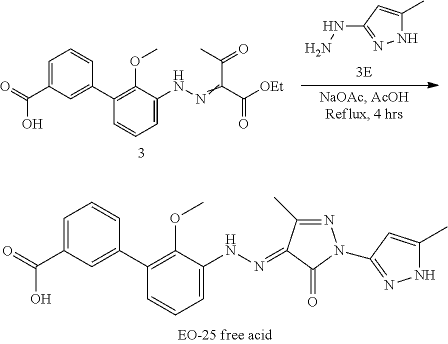

To a solution of 3 (1.40 g, 3.64 mmol, 1.00 eq) and (4,5-dimethylthiazol-2-yl)hydrazine (510 mg, 4.55 mmol, 1.25 eq) in AcOH (35.0 mL) was added NaOAc (364 mg, 4.44 mmol, 1.22 eq) in one portion at 25° C. under N₂. And then stir the mixture at 120° C. for 3 hrs. LCMS showed the reaction was completed and the product was detected (RT=1.76 mins). The mixture was cooled to 25° C. The mixture filtered, washed with water (10.0 mL). Concentrated the mixture was to give crude product. Compound EO-25 free acid (1.25 g, 79.6% yield) was obtained as red solid.

LCMS: EC457-59-P1E, RT=1.76 mins, M+1=471

### General Procedure for Preparation of Compound EO-25

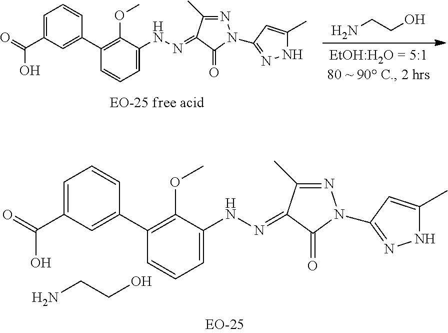

To a solution of EO-25 freed acid (80.0 mg, 181 μmol, 1.00 eq) and in EtOH:H2O=5:1 (5.00 mL) was added 2-aminoethanol (13.3 mg, 217 μmol, 13.1 uL, 1.20 eq) in one portion at 25° C. under N₂ protect. HPLC showed the reaction was completed. The product was detected (RT=1.91 mins). The mixture was stirred at 80° C. for 2 hrs. Concentrated the mixture was to give product. The product EO-25 (86.0 mg, 94.4% yield) was obtained as yellow oil.

LCMS: EC457-67-P1F, product: RT=1.776 min, M+1=433

¹H NMR: EC457-67-P1C DMSO

δ 8.11 (s, 1H), 7.71-7.93 (d, 1H), 7.15 (s, 1H), 7.63-7.71 (d, 1H), 7.40-7.49 (d, 1H), 7.32 (s, 1H), 7.21 (s, 1H), 6.24 (s, 1H), 7.02-7.11 (m, 2H), 2.22-2.32 (m, 3H), 7.02-7.11 (t, 2H), 2.21 (s, 3H), 2.20 (s, 3H).

1. A compound of Formula (I): 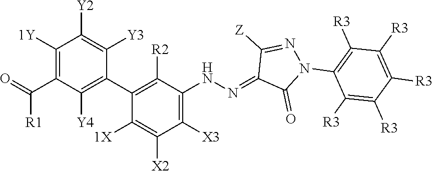
or
Formula (II): 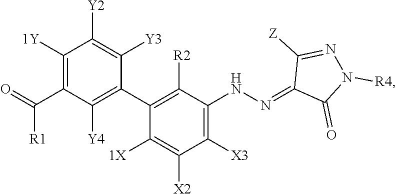
or a stereoisomer or a pharmaceutically acceptable salt thereof,
wherein:
R1 is selected from OH, O—CH₃, O—CH₂CH₃, O—CH(CH₃)₂, NH—CH₃, and NH—CH₂CH₃;
R2 is selected from H, F, Cl, CH₃, CF₃, OCH₃, CH₂CH₃, OCH₂CH₃;
R3 is selected from H, OH, CH₃, CF₃, NH₂, F, Cl, OCH₃, and NHCOCH₃;
X1, X2, or X3 is selected from H, F, Cl, OH, CH₃, CF₃, CH₂CH₃, OCH₃, OCH₂CH₃, and —CO—CH₃;
Y1, Y2, Y3, or Y4 is selected from H, F, Cl, CH₃, CF₃, OCH₃, and CH₂CH₃;
Z is selected from H, F, Cl, CH₃, CF₃, OCH₃, CH₂CH₃, CH₂NH₂, and CH₂CH₂NH₂;
R4 is selected from: 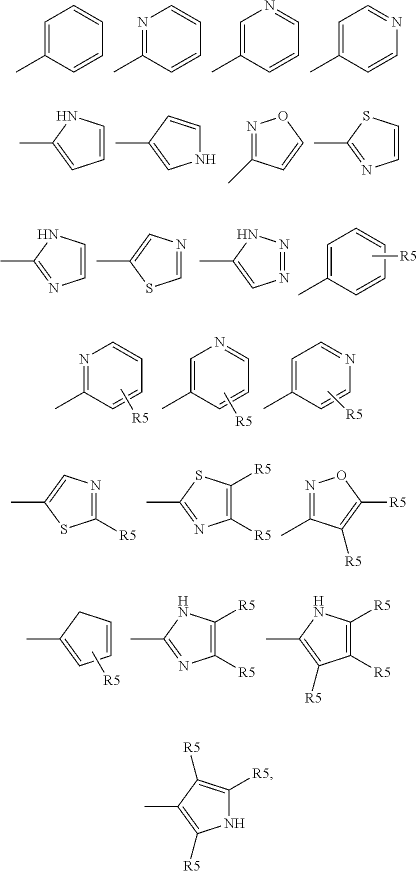
R5 is selected from H, OH, CH₃, CF₃, NH₂, F, Cl, OCH₃, and NHCOCH₃.

2. The compound of claim 1 selected from:

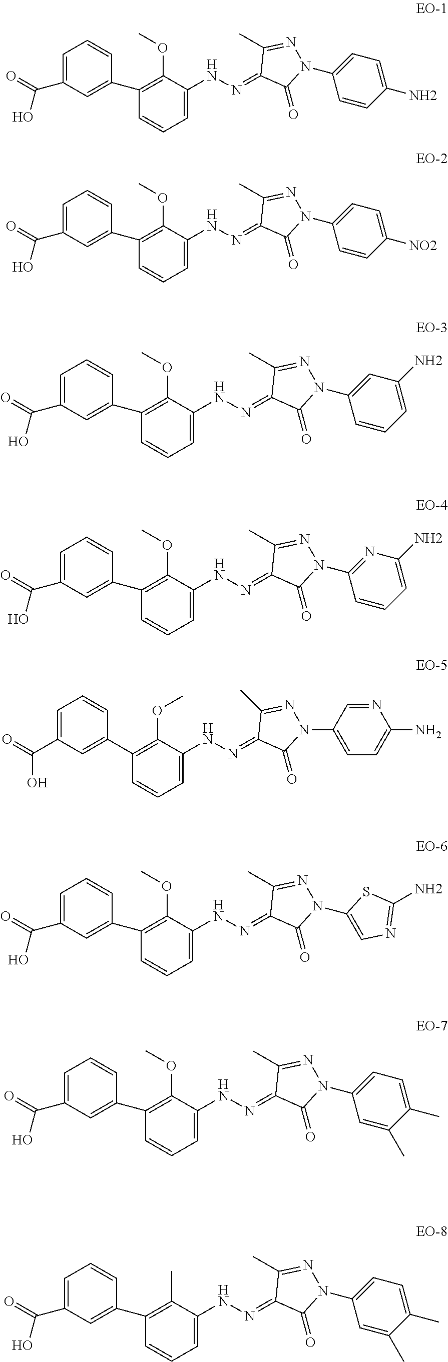

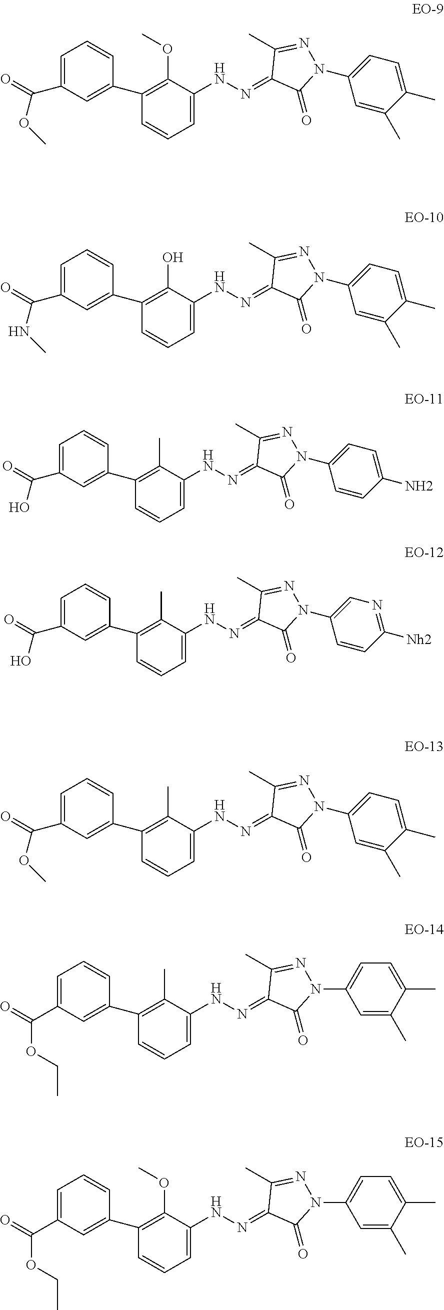

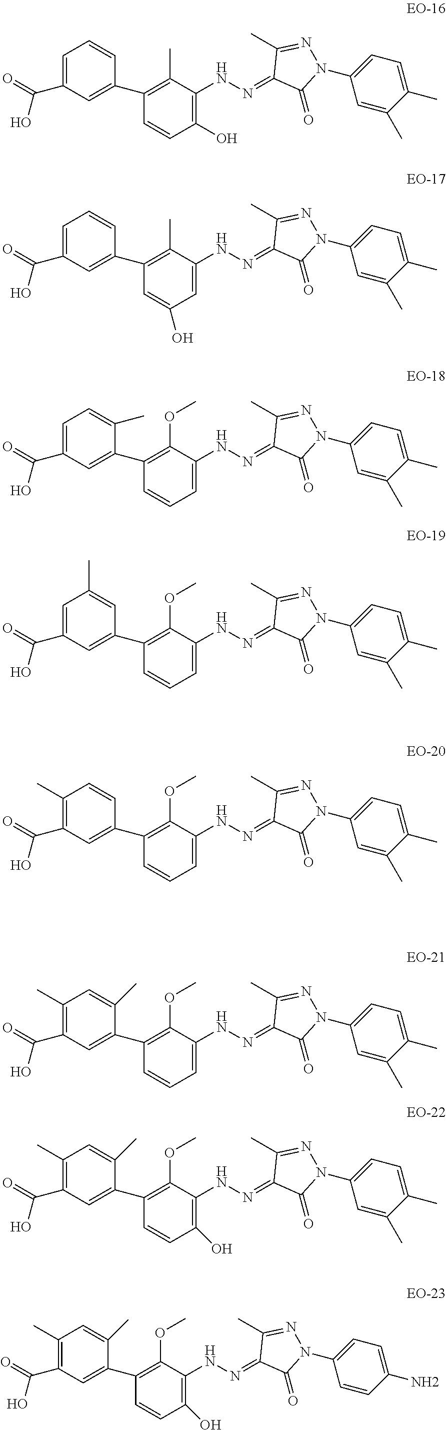

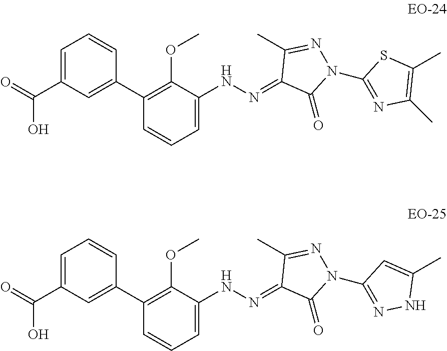 [SMILES: COc1c(N/N=C2/C(=O)N(c3nc(C)c(C)s3)N=C2C)cccc1-c1cccc(C(=O)O)c1 ; COc1c(N/N=C2/C(=O)N(c3cc(C)[nH]n3)N=C2C)cccc1-c1cccc(C(=O)O)c1]

3. A pharmaceutical composition comprising (i) the compound of claim 1, or a stereoisomer or a pharmaceutically acceptable salt thereof, and (ii) a pharmaceutically acceptable carrier.

4. A method of treating or preventing a disorder mediated by BAX in a subject, comprising administering to the subject a therapeutically effective amount of the compound of claim 1 or a stereoisomer or a pharmaceutically acceptable salt thereof.

5. The method of claim 4, wherein the disorder is associated with increased expression or activation of the BAX protein.

6. The method of claim 4, wherein the disorder comprises a neuronal disorder or an autoimmune disease.

7. The method of claim 6, wherein the neuronal disorder is selected from epilepsy, multiple sclerosis, Alzheimer's disease, Huntington's disease, Parkinson's disease, retinal diseases, spinal cord injury, Crohn's disease, head trauma, spinocerebellar ataxias, and dentatorubral-pallidoluysian atrophy.

8. The method of claim 6, wherein the autoimmune disease is selected from Multiple Sclerosis, amyotrophic lateral sclerosis, retinitis pigmentosa, inflammatory bowel disease (IBD), rheumatoid arthritis, asthma, septic shock, transplant rejection, and AIDS.

9. The method of claim 4, wherein the disorder is selected from ischemia, cardiomyopathy, cardiovascular disorders, myocarditis, arteriosclerosis, heart failure, heart transplantation, acute liver injury, acute kidney injury, renal hypoxia, acute optic nerve damage, Idiopathic pulmonary fibrosis (IPF), glaucoma and hepatitis.

10. The method of claim 4, wherein the disorder is selected from chemotherapy-induced cardiotoxicity, chemotherapy-induced cardiomyopathy, chemotherapy-induced liver injury, chemotherapy-induced kidney injury, chemotherapy-induced ocular toxicity.

11. The method of claim 4, further comprising administering to the subject a second therapeutic agent or therapy.

12. The method of claim 11, wherein the second therapeutic agent comprises an anti-inflammatory agent or an anti-tumor/anti-cancer agent.

13. The method of claim 12, wherein the anti-tumor/anti-cancer agent is navitoclax.

14. The method of claim 11, wherein the second therapeutic agent is administered to the subject before, after, or concurrently with the compound of claim 1 a stereoisomer or a pharmaceutically acceptable salt thereof.

15. The method of claim 4, wherein the subject was previously administered an anti-cancer therapy.

16. The method of claim 15, wherein the anti-cancer therapy comprises surgery, radiation, chemotherapy, and/or immunotherapy.

17. The method of claim 16, wherein the chemotherapy comprises a therapeutic agent that inhibits Bcl-xL.

18. The method of claim 17, wherein the therapeutic agent that inhibits Bcl-xL comprises navitoclax.

19. The method of claim 4, wherein the subject is a mammal.

20. The method of claim 4, wherein the subject is a human.

21. The method of claim 4, wherein the compound of claim 1 or a stereoisomer or a pharmaceutically acceptable salt thereof, is administered intratumorally, intravenously, subcutaneously, intraosseously, orally, transdermally, in sustained release, in controlled release, in delayed release, as a suppository, or sublingually.

22. The method of claim 4, wherein the compound of claim 1 or a stereoisomer or a pharmaceutically acceptable salt thereof, is administered prophylactically or therapeutically.

23. A method of treating or ameliorating a symptom of thrombocytopenia associated with treatment targeting Bcl-xL, comprising:
(i) selecting a subject having a condition treatable by a therapeutic agent that inhibits Bcl-xL; and
(ii) administering to the subject a therapeutically effective amount of the compound of claim 1 or a stereoisomer or a pharmaceutically acceptable salt thereof, in combination with a therapeutically effective amount of the therapeutic agent.

24. The method of claim 23, wherein the therapeutic agent that inhibits Bcl-xL comprises navitoclax.

25. The method of claim 23, wherein the condition is a cancer or an autoimmune disease.

26. The method of claim 23, wherein the therapeutic agent is administered to the subject before, after, or concurrently with the compound of claim 1 or a stereoisomer or a pharmaceutically acceptable salt thereof.

27. The method of claim 23, wherein the therapeutic agent or the compound of claim 1 or a stereoisomer or a pharmaceutically acceptable salt thereof, is administered to the subject in one or more doses.

28. A method of inhibiting BAX-mediated apoptosis in a cell, comprising administering to the cell expressing a BAX protein an effective amount of the compound of claim 1 or a stereoisomer or a pharmaceutically acceptable salt thereof.

29. A method of inhibiting activation or function of the BAX protein in a subject, a cell, or a biological sample thereof, comprising (i) administering to the subject or the cell a therapeutically effective amount of the compound of claim 1 or a stereoisomer or a pharmaceutically acceptable salt thereof; or (ii) contacting the biological sample with the compound of claim 1 or a stereoisomer or a pharmaceutically acceptable salt thereof.

30. The method of claim 28, wherein the cell is a neuronal cell or a cardiac cell.

31. The method of claim 29, wherein the activation of BAX protein is mediated by Bim, Bid, Bmf, Puma, or Noxa.

32. A method for preserving or treating an organ or tissue, comprising contacting the organ or tissue with an effective amount of the compound of claim 1 or a stereoisomer or a pharmaceutically acceptable salt thereof, under conditions effective for preservation of the organ or tissue.


SEQUENCE LISTING (3 sequences)

SEQ ID NO: 1 - protein, 20 aa, Artificial
  Synthetic
  S5 (10)..(11): Hydrocarbon-stapled peptide corresponding to the BH3 domain of BIM, BIM SAHBA2, where S5 represents the non-natural amino acid inserted for olefin metathesis
  S5 (15)..(16): Hydrocarbon-stapled peptide corresponding to the BH3 domain of BIM, BIM SAHBA2, where S5 represents the non-natural amino acid inserted for olefin metathesis
EIWIAQELRSIGDSFNAYYA

SEQ ID NO: 2 - protein, 24 aa, Artificial
  Synthetic
RPEIWIAQELRRIGDEFNAYYARR

SEQ ID NO: 3 - protein, 192 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG